SEQUENCE LISTING (23 sequences)

SEQ ID NO: 1 - protein, 315 aa, Synthetic construct
MLKKIISLYKRYSISKKLVLDNEHFIKENKNIYGKKHKGFFDFDEKAKDVKSPLNPWGFIRVKNEALTLRVSLESILPALQRGIIAYNDCDDGSEELILEFCKQYPNFIAKKYPYKVDLENPKNEENKLYSYYNWAASFIPLDEWFIKIDVDHYYDAKKLYKSFYRIDQENKALCYPRINFIILNGNIYVQNSGNYGFIGGGDQLLIKRRNSSFIERRVSKKSQWIDPKGLIEELYSEQQVLSQGVKILQAPLLQWHFPALKYRRNDYQQYLDILSLEEFQAFHRKSKEAKKIDFAMLKRPVIEQILKKFQGEIK

SEQ ID NO: 2 - protein, 300 aa, Synthetic construct
KKLVLDNEHFIKENKNIYGKKHKGFFDFDEKAKDVKSPLNPWGFIRVKNEALTLRVSLESILPALQRGIIAYNDCDDGSEELILEFCKQYPNFIAKKYPYKVDLENPKNEENKLYSYYNWAASFIPLDEWFIKIDVDHYYDAKKLYKSFYRIDQENKALCYPRINFIILNGNIYVQNSGNYGFIGGGDQLLIKRRNSSFIERRVSKKSQWIDPKGLIEELYSEQQVLSQGVKILQAPLLQWHFPALKYRRNDYQQYLDILSLEEFQAFHRKSKEAKKIDFAMLKRPVIEQILKKFQGEIK

SEQ ID NO: 3 - protein, 271 aa, Synthetic construct
MFKISIILPTYNVEQYIARAIESCIKQTFKDIEIIVVDDCGNDNSINIAKEYSKKDKRIKIIHNEKNIGLLRARYEGVKVANSPYIMFLDPDDYLELNACEECIKILDEQDEVDLVFFNAIVESNVISYKKFDFNSGFYSKKEFVKKIIAEKNLYWTMWGKLIRKKLYLEAFASLRLEKDVKINMAEDVLLYYPMLSQAQKIAYMNCNLYHYVPNNNSICNTKNEVGVKNNIQDLQLVLNYLRQNYILNKYCSVLYVLIKYLLYIQIYKIK

SEQ ID NO: 4 - DNA, 2094 bp, Synthetic construct
  source 1..2094: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcaagaaactggtgctggacaacgagcactttattaaggaaaacaaaaacatctacggcaagaagcacaagggtttcttcgatttcgatgagaaggcaaaggatgtgaagagcccgctgaatccgtggggttttattcgtgttaagaatgaagcactgaccctgcgtgttagcctggaaagcattctgccagccctgcaacgtggcattattgcgtataatgactgtgatgatggcagcgaagaactgatcctggagttttgcaagcagtacccgaactttatcgccaaaaagtatccgtacaaggtggacctggagaacccgaaaaacgaagagaacaaactgtacagctactataattgggcggcaagttttattccgctggatgagtggttcatcaagatcgacgtggaccattactatgacgcgaaaaagctgtataagagcttctaccgcattgaccaggaaaataaggcgctgtgttacccacgcatcaacttcatcatcctgaacggcaacatttacgtgcaaaatagcggtaactatggtttcattggtggcggtgaccaactgctgatcaaacgtcgtaatagcagcttcatcgagcgtcgcgttagcaaaaaaagccagtggatcgacccgaagggtctgattgaggaactgtatagcgaacagcaggtgctgagccaaggcgtgaaaattctgcaggcgccactgctgcaatggcacttcccggcactgaaatatcgccgcaatgactaccagcaatacctggatattctgagcctggaggaatttcaggccttccaccgtaagagcaaggaagcaaagaaaatcgactttgccatgctgaagcgcccagttattgagcagattctgaagaagtttcagggtgagatcaaacaccaccaccaccaccactaa

SEQ ID NO: 5 - protein, 697 aa, Synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFKKLVLDNEHFIKENKNIYGKKHKGFFDFDEKAKDVKSPLNPWGFIRVKNEALTLRVSLESILPALQRGIIAYNDCDDGSEELILEFCKQYPNFIAKKYPYKVDLENPKNEENKLYSYYNWAASFIPLDEWFIKIDVDHYYDAKKLYKSFYRIDQENKALCYPRINFIILNGNIYVQNSGNYGFIGGGDQLLIKRRNSSFIERRVSKKSQWIDPKGLIEELYSEQQVLSQGVKILQAPLLQWHFPALKYRRNDYQQYLDILSLEEFQAFHRKSKEAKKIDFAMLKRPVIEQILKKFQGEIKHHHHHH

SEQ ID NO: 6 - DNA, 2004 bp, Synthetic construct
  source 1..2004: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcttcaaaatttctatcatcctgccgacctataacgtggaacagtatatcgctcgcgcaatcgagagctgcatcaaacagaccttcaaggacatcgagattatcgtggtggacgactgcggcaacgataactccatcaacattgctaaggaatacagcaagaaggacaagcgtatcaaaatcattcacaacgaaaagaacatcggcctgctgcgtgcacgttacgaaggtgttaaagtggcgaacagcccgtacatcatgttcctggacccggacgattatctggagctgaacgcgtgcgaagagtgcattaagattctggacgagcaggacgaagtggacctggtgttcttcaacgctattgtggagtccaacgtgatcagctacaagaaattcgattttaactccggcttctactccaaaaaggagttcgtgaaaaagatcatcgcagagaagaacctgtactggaccatgtggggcaaactgattcgcaagaaactgtacctggaggcgttcgcttccctgcgtctggaaaaggatgttaagatcaacatggcggaagacgttctgctgtactacccgatgctgtctcaggcacagaaaattgcgtacatgaactgcaacctgtaccactatgtgccgaacaacaactctatttgcaacaccaaaaacgaggtgggcgttaaaaacaacattcaggacctgcagctggtgctgaactacctgcgccagaactacatcctgaacaagtactgcagcgttctgtacgtgctgattaagtacctgctgtatattcagatctacaaaattaagcatcatcatcaccaccactaa

SEQ ID NO: 7 - protein, 667 aa, Synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFFKISIILPTYNVEQYIARAIESCIKQTFKDIEIIVVDDCGNDNSINIAKEYSKKDKRIKIIHNEKNIGLLRARYEGVKVANSPYIMFLDPDDYLELNACEECIKILDEQDEVDLVFFNAIVESNVISYKKFDFNSGFYSKKEFVKKIIAEKNLYWTMWGKLIRKKLYLEAFASLRLEKDVKINMAEDVLLYYPMLSQAQKIAYMNCNLYHYVPNNNSICNTKNEVGVKNNIQDLQLVLNYLRQNYILNKYCSVLYVLIKYLLYIQIYKIKHHHHHH

SEQ ID NO: 8 - DNA, 28 bp, Synthetic construct
  source 1..28: mol_type = other DNA
gaccgaattcgtgctggacaacgagcac

SEQ ID NO: 9 - DNA, 28 bp, Synthetic construct
  source 1..28: mol_type = other DNA
cagcaagctttcagtggtggtggtggtg

SEQ ID NO: 10 - DNA, 34 bp, Synthetic construct
  source 1..34: mol_type = other DNA
gaccgaattcttcaaaatttctatcatcctgccg

SEQ ID NO: 11 - DNA, 56 bp, Synthetic construct
  source 1..56: mol_type = other DNA
cagcaagcttttagtggtggtgatgatgatgcttaattttgtagatctgaatatac

SEQ ID NO: 12 - protein, 300 aa, Synthetic construct
KKLVLDNEHFIKENKNIYGKKHKGFFDFDEKAKDPKSPLNPWAFIRVKNEALTLRVSLESILPALQRGIIAYNDCDDGSEEIILEFCKQYPSFIPKKYPYKVDLENPKNEENKLYSYYNWAASFIPQDEWFIKIDVDHYYDAKKLYKSFYMIDQENKALCYPRINFIILNGNIYVQNSGNYGFIGGGDQLLIKRRNSSFIERRVSKKSQWIDPTGLIEELYSYQQVLSKGVKILQAPLLQWHFPALKYRRNDYQQYLDILSLEEFQAFHRKSKEAKKIDFAMLKREVIEQILKKFQGEIK

SEQ ID NO: 13 - protein, 300 aa, Synthetic construct
KKLVLDNEHFIKENKNIYGKKHKGFFDFDEKAKDPKSPLNPWAFIRVKNEALTLRVSLESILPALQRGIIAYNDCDDGSEEIILEFCKQYPSFIPVKYPYKVQLENPKNEENKLYSYYNWAASFIPQDEWFIKIDVDHYYDAKKLYKSFYMIDQENKALCYPRINFIILNGNIYVQNSGNYGFIGGGDQLLIKRRNSSFIERMVSKKSQWIDPTGLINELYSYQQVLSKGVKILQAPLLQWHFPALKYRRNDYQQYLDILSLEEFQRFHRKSKEAKKIDFAMLKREVIEQILKKFQGEIK

SEQ ID NO: 14 - protein, 300 aa, Synthetic construct
KKLVLDNEHFIKENKNIYGKKHKGFFDFDEKAKDPKSPLNPWAFIRVKNEALTLRVSLESILPAIQRGIIAYNDCDDGSEEIILEFCKQYPSFIPVKYPYKVQLENPKNEENKLYSYYNWVASFIPQDEWFIKIDVDHYYDAKKLYKSFYMIDQDNKALCYPRINFIILNGNIYVQNSGNYGFIGGGDQLLIKNRNSKFVERMVSKKSQWIDPTGTINELYSYQQVLSKGVKILQAPLLQWHFPALKYRRNDYQQYLDILSLEEFQRFHRKSKEAKKIDFSMLKREVIEQILKKFQGEIK

SEQ ID NO: 15 - DNA, 900 bp, Synthetic construct
  source 1..900: mol_type = other DNA
aagaaactggttcttgacaatgaacattttataaaggagaataagaatatttacgggaagaagcacaagggattcttcgacttcgatgagaaggcaaaagaccctaagtctccgctgaacccatgggccttcattcgcgtaaagaatgaggcactcaccttacgcgtctcattggagagcatactcccagccttacaaagaggaatcatagcctataacgactgtgatgatggaagcgaagaaatcatattggagttctgcaagcagtacccgagttttatccctaagaagtacccctacaaagtggacttggagaacccgaagaacgaagaaaataagttatattcatactacaattgggccgcctctttcatcccgcaggacgagtggtttatcaagatagacgtggaccattattacgacgcgaagaagctgtacaagagcttctacatgatagaccaggaaaataaggctctgtgttatccacgtatcaactttataatcttgaacggtaacatctacgtgcagaatagtggtaactacggcttcattggaggaggtgaccagctcctgattaagcggcgcaatagttcgttcatcgaacgacgtgtatcaaagaagtctcaatggattgacccaacgggtttaatcgaggaactgtattcctatcaacaggttctgagcaagggtgtcaaaatcctgcaagctcctctgttgcaatggcacttccccgccttgaagtatcggcggaatgactaccaacaatacctcgacatcttgagcctggaagagttccaagcctttcatcgcaagagtaaggaagcaaagaagatagattttgcgatgctgaagcgggaagtaatagaacagatcctgaaaaaattccagggtgagatcaaa

SEQ ID NO: 16 - DNA, 900 bp, Synthetic construct
  source 1..900: mol_type = other DNA
aagaaactggttcttgacaatgaacattttattaaggaaaacaagaacatttatggcaagaagcacaaaggattcttcgacttcgacgaaaaggccaaggacccgaagagccccttaaacccgtgggcatttatacgcgttaagaatgaagcactgacgttaagagttagtttggaaagtatccttccggcgctgcaacggggtattatagcttataacgactgtgacgacggaagcgaagagataatactggagttctgtaagcagtacccttcattcataccagtaaaatatccgtacaaagtccagcttgagaatccgaagaacgaagagaataagttatattcttactacaattgggccgccagcttcatcccacaagatgagtggttcatcaagatagacgttgaccattattatgacgcaaagaaactctacaagtcattctacatgatagatcaggagaataaagcgttatgctaccctcgaatcaactttatcatccttaatggtaacatatacgtccaaaacagcggcaactacggcttcatcggcggtggggatcaactcctgatcaagcgtagaaatagctcgtttatagagcgcatggttagcaagaaaagccagtggattgacccgaccggtctcataaatgagttgtacagctaccagcaagttttgtccaaaggtgtcaaaattctccaagccccgctgctgcaatggcatttcccagcccttaagtaccgtcgcaacgactatcaacagtatctcgacatacttagccttgaagagtttcaacgtttccacagaaagagtaaagaggctaagaagattgacttcgcaatgttaaagcgggaagtcatcgaacagatcctgaaaaaattccagggtgagatcaaa

SEQ ID NO: 17 - DNA, 900 bp, Synthetic construct
  source 1..900: mol_type = other DNA
aagaaactggttcttgacaatgaacattttatcaaggaaaacaagaacatctacggcaagaaacacaagggcttcttcgacttcgacgagaaggcgaaagaccctaagagccctttaaacccttgggcattcattcgggtaaagaacgaggcgctgactttgagagtcagcctggagagtatacttcctgccatacaacgcggtataatcgcatataatgactgcgacgacggatcagaagaaatcatccttgaattttgcaagcaataccctagcttcatcccagtgaagtatccgtataaagtacaacttgagaacccaaagaatgaggagaataagttatattcgtattataactgggtggcctctttcataccccaggatgagtggttcataaagatcgatgttgaccactactacgacgctaagaagctgtacaagtctttctatatgatcgaccaagataacaaggctttatgctacccacgtataaatttcataatcttgaatgggaatatctacgtgcaaaactcaggcaactacgggtttatcggtggtggcgatcaactgctgattaagaatcgcaattccaagtttgttgagcgtatggtatcaaagaaaagccaatggattgatccgactgggaccattaacgagttatactcttaccaacaggttctcagcaaaggggtgaagattcttcaagcgcccttactgcaatggcatttccctgcactcaagtaccgtagaaatgactatcaacaatacctggatattctttctttggaagagttccaaagatttcaccgcaagtctaaggaagccaagaaaattgacttttctatgctgaagcgtgaagtcatagagcaaatcctgaaaaaattccagggtgagatcaaa

SEQ ID NO: 18 - DNA, 32 bp, Synthetic construct
  source 1..32: mol_type = other DNA
gaccgaattcaagaaactggttcttgacaatg

SEQ ID NO: 19 - DNA, 47 bp, Synthetic construct
  source 1..47: mol_type = other DNA
cagcaagcttttagtggtggtgatgatgatgtttgatctcaccctgg

SEQ ID NO: 20 - DNA, 23 bp, Synthetic construct
  source 1..23: mol_type = other DNA
actgtattccgagcaacaggttc

SEQ ID NO: 21 - DNA, 19 bp, Synthetic construct
  source 1..19: mol_type = other DNA
tcctcgattaaacccgttg

SEQ ID NO: 22 - DNA, 2094 bp, Synthetic construct
  source 1..2094: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcaagaaactggttcttgacaatgaacattttataaaggagaataagaatatttacgggaagaagcacaagggattcttcgacttcgatgagaaggcaaaagaccctaagtctccgctgaacccatgggccttcattcgcgtaaagaatgaggcactcaccttacgcgtctcattggagagcatactcccagccttacaaagaggaatcatagcctataacgactgtgatgatggaagcgaagaaatcatattggagttctgcaagcagtacccgagttttatccctaagaagtacccctacaaagtggacttggagaacccgaagaacgaagaaaataagttatattcatactacaattgggccgcctctttcatcccgcaggacgagtggtttatcaagatagacgtggaccattattacgacgcgaagaagctgtacaagagcttctacatgatagaccaggaaaataaggctctgtgttatccacgtatcaactttataatcttgaacggtaacatctacgtgcagaatagtggtaactacggcttcattggaggaggtgaccagctcctgattaagcggcgcaatagttcgttcatcgaacgacgtgtatcaaagaagtctcaatggattgacccaacgggtttaatcgaggaactgtattccgagcaacaggttctgagcaagggtgtcaaaatcctgcaagctcctctgttgcaatggcacttccccgccttgaagtatcggcggaatgactaccaacaatacctcgacatcttgagcctggaagagttccaagcctttcatcgcaagagtaaggaagcaaagaagatagattttgcgatgctgaagcgggaagtaatagaacagatcctgaaaaaattccagggtgagatcaaacaccaccaccaccaccactga

SEQ ID NO: 23 - protein, 697 aa, Synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFKKLVLDNEHFIKENKNIYGKKHKGFFDFDEKAKDPKSPLNPWAFIRVKNEALTLRVSLESILPALQRGIIAYNDCDDGSEEIILEFCKQYPSFIPKKYPYKVDLENPKNEENKLYSYYNWAASFIPQDEWFIKIDVDHYYDAKKLYKSFYMIDQENKALCYPRINFIILNGNIYVQNSGNYGFIGGGDQLLIKRRNSSFIERRVSKKSQWIDPTGLIEELYSEQQVLSKGVKILQAPLLQWHFPALKYRRNDYQQYLDILSLEEFQAFHRKSKEAKKIDFAMLKREVIEQILKKFQGEIKHHHHHH

(19) United States
(12) Patent Application Publication
(10) Pub. No.: US 2026/0258378 A1
(43) Pub. Date: Sep. 3, 2026

(54) GLYCOSYLTRANSFERASE ENGINEERING FOR CHEMOENZYMATIC TOTAL SYNTHESIS OF GANGLIOSIDES

(71) Applicant: The Regents of the University of California, Oakland, CA (US)

(72) Inventors: Xi CHEN, Davis, CA (US); Hai YU, Woodland, CA (US); Libo ZHANG, Davis, CA (US); Arin GUCCHAIT, Davis, CA (US)

(21) Appl. No.: 19/126,098

(22) PCT Filed: Nov. 1, 2023
(86) PCT No.: PCT/US2023/078398

§ 371 Date: Apr. 30, 2025

Related U.S. Application Data
(60) Provisional application No. 63/421,284, filed on Nov. 1, 2022.

(51) Int. Cl. C12N 9/10; C12P 19/04; C12P 19/18

(52) U.S. Cl. CPC C12N 9/1051; C12P 19/04; C12P 19/18; C12Y 204/01062; C12Y 204/01092

(57) ABSTRACT

Described herein are Campylobacter jejuni β1-4GalNAcT (CjCgtA) variants comprising a polypeptide having at least 80% identity to the amino acid sequence set forth in SEQ ID NO: 1 or SEQ ID NO: 2. Also described herein are Campylobacter jejuni β1-3-galactosyltransferase (CjCgtB) variants comprising a polypeptide having at least 80% identity to the amino acid sequence set forth in SEQ ID NO: 3. Optionally, in the CjCgtA and CjCgtB variants, the N-terminus of the polypeptide is fused to a maltose binding protein.

Further described herein is a method for preparing a glycosylated molecule, comprising: forming a reaction mixture comprising (i) a glycosylation donor comprising a sugar component; (ii) a glycosylation acceptor comprising a sphingosine moiety; and (iii) a glycosyltransferase, wherein the glycosyltransferase is a CjCgtA variant as described herein or a CjCgtB variant as described herein; and maintaining the reaction mixture under conditions suitable for forming a glycosylated molecule.

### CROSS-REFERENCE TO RELATED APPLICATION

The application claims the benefit of and the priority to U.S. Provisional Application No. 63/421,284, filed Nov. 1, 2022, which is hereby incorporated by reference in its entirety for all purposes.

### STATEMENT REGARDING FEDERALLY FUNDED RESEARCH

This invention was made with government support under Grant Nos. U01GM120419 and R44GM139441, awarded by the National Institutes of Health. The government has certain rights in the invention.

### REFERENCE TO BIOLOGICAL SEQUENCE DISCLOSURE

The instant application contains a Sequence Listing which has been submitted electronically in XML file format and is hereby incorporated by reference in its entirety. Said XML copy, created on Oct. 31, 2023, is named 076916-1414071.xml and is 33,672 bytes in size.

### BACKGROUND

GM1a, or more commonly named as GM1, is an important member of sialic acid-containing glycosphingolipids (GSLs) called gangliosides. The structure of GM1, Galβ3GalNAcβ4(Neu5Acα3)Galβ4Glcβ-ceramide consists of a sialic acid-containing pentasaccharide linked via a β-glycosidic bond to a special type of lipid called ceramide. In human and other mammals, the ceramide contains a sphingosine attached with a fatty acyl chain via an amide bond. Gangliosides are presented in the outer leaflet of the plasma membrane of different cell types but are the most abundant in those of the nervous system. GM1, and its more highly sialylated counterparts including GD1a, GD1b, and GT1b, constitute the four major gangliosides in human and animal brains. While GM1 in the brains of both humans and animals contains mainly the most common sialic acid form, N-acetylneuraminic acid (Neu5Ac), GM1 containing a non-human sialic acid form N-glycolylneuraminic acid (Neu5Gc) has been found in bovine brains.

The important roles of GM1 and other gangliosides are well known. Specific ganglioside-binding domains (GBDs) have been identified in various proteins including neurotransmitter receptors, bacterial toxins, viral surface proteins, and proteins related to the cause of various neurodegenerative diseases. Recently, SARS-CoV-2 receptor binding domain (RBD) was shown to bind to GM1, GM2, and GM3. The therapeutic potential of exogenously admitted gangliosides in treating patients with the Rett Syndrome, Huntington's Disease (HD), and Parkinson's Disease (PD) is emerging. More specifically, the neurotrophic and neuroprotective effects of GM1 have been identified. Recently, GM1 as well as GD3, GD1a, GD1b, and GT1b, but not GM3 or GQ1b, were shown to decrease inflammatory microglia responses in vitro and in vivo. GM1 or GM1-containing gangliosides purified from animal brains have been used as medicines for treating peripheral neuropathies, brain and spinal cord injuries, and are being developed as potential drugs for treating HD and PD. The GM1 oligosaccharide (OligoGM1) is also emerging as a potential candidate for treating PD. In addition, GM1 micelles and GM1 sphingosine (or lysoGM1) have been used to develop drug delivery vesicles with or without poly(lactic-co-glycolic acid) (PLGA). They have been shown to be able to cross the brain blood barrier (BBB).

### SUMMARY

Described herein is a Campylobacter jejuni β1-4GalNAcT (CjCgtA) variant comprising a polypeptide having at least 80% identity to the amino acid sequence set forth in SEQ ID NO: 1. Optionally, the variant comprises a mutation at one or more positions corresponding to G58, A110, and R166 in SEQ ID NO: 1. The CjCgtA variant can further comprise a mutation at P301 in SEQ ID NO: 1; a mutation at one or more positions corresponding to L97 and Q244 in SEQ ID NO: 1; a mutation at one or more positions corresponding to L142 and K229 in SEQ ID NO: 1; a mutation at N107 in SEQ ID NO: 1; a mutation at V50 in SEQ ID NO: 1; a mutation at one or more positions corresponding to Q169, S212, S213, and A282 in SEQ ID NO: 1; a mutation at G200 in SEQ ID NO: 1; a mutation at one or more positions corresponding to D 118, K286, and A296 in SEQ ID NO: 1; a mutation at S287 in SEQ ID NO: 1; a mutation at S243 in SEQ ID NO: 1; a mutation at S193 in SEQ ID NO: 1; a mutation at N124 in SEQ ID NO: 1; a mutation at L80 in SEQ ID NO: 1; a mutation at K46 in SEQ ID NO: 1; a mutation at K288 in SEQ ID NO:1; a mutation at K35 in SEQ ID NO: 1; a mutation at one or more positions corresponding to E170, F214, and I215 in SEQ ID NO: 1; and/or a mutation at one or more positions corresponding to K111, S131, V190, R209, R210, V246, E289, and E304 in SEQ ID NO: 1. Optionally, the CjCgtA variant comprises a mutation at one or more positions corresponding to K35, K46, V50, G58, L80, L97, N107, A110, K111, D118, N124, S131, L142, R166, Q169, E170, V190, S193, G200, R209, R210, S212, S213, F214, I215, R218, K229, L231, S243, Q244, V246, A282, K286, S287, K288, E289, A296, P301, and/or E304 in SEQ ID NO: 1.

Also described herein is a Campylobacter jejuni β1-4GalNAcT (CjCgtA) variant comprising a polypeptide having at least 80% identity to the amino acid sequence set forth in SEQ ID NO: 2. Optionally, the variant comprises a mutation at one or more positions corresponding to G43, A95, and R151 in SEQ ID NO: 2. The CjCgtA variant can further comprise a mutation at P286 in SEQ ID NO: 2; a mutation at one or more positions corresponding to L82 and Q229 in SEQ ID NO: 2; a mutation at one or more positions corresponding to L127 and K214 in SEQ ID NO: 2; a mutation at N92 in SEQ ID NO: 2; a mutation at V35 in SEQ ID NO: 2; a mutation at one or more positions corresponding to Q154, S197, S198, and A267 in SEQ ID NO: 2; a mutation at G185 in SEQ ID NO: 2; a mutation at one or more positions corresponding to D103, K271, and A281 in SEQ ID NO: 2; a mutation at S272 in SEQ ID NO: 2; a mutation at S228 in SEQ ID NO: 2; a mutation at S178 in SEQ ID NO: 2; a mutation at N109 in SEQ ID NO: 2; a mutation at L65 in SEQ ID NO: 2; a mutation at K31 in SEQ ID NO: 2; a mutation at K273 in SEQ ID NO: 2; a mutation at K20 in SEQ ID NO: 2; a mutation at one or more positions corresponding to E155, F199, and 1200 in SEQ ID NO: 2; and/or a mutation at one or more positions corresponding to K96, S116, V175, R194, R195, V231, E274, and E289 in SEQ ID NO: 2. Optionally, the CjCgtA variant comprises a mutation at one or more positions corresponding to K20, K31, V35, G43, L65, L82, N92, A95, K96, D103, N109, S116, L127, R151, Q154, E155, V175, S178, G185, R194, R195, S197, S198, F199, I200, R203, K214, L216, S228, Q229, V231, A267, K271, S272, K273, E274, A281, P286, and/or E289 in SEQ ID NO: 2.

Also described herein is a Campylobacter jejuni β1-4GalNAcT (CjCgtA) variant comprising a polypeptide having at least 80% identity to the amino acid sequence set forth in SEQ ID NO: 23. Further described herein is a Campylobacter jejuni β1-4GalNAcT (CjCgtA) variant comprising a polypeptide having the amino acid sequence set forth in SEQ ID NO: 23.

Optionally, in the CjCgtA variant, the N-terminus of the polypeptide is fused to a maltose binding protein.

Also described herein is a Campylobacter jejuni β1-3-galactosyltransferase (CjCgtB) variant comprising a polypeptide having at least 80% identity to the amino acid sequence set forth in SEQ ID NO: 3. Optionally, the CjCgtB variant comprises a mutation at one or more positions corresponding to K26, S53, and K166 in SEQ ID NO: 3. The CjCgtB variant can further comprise a mutation at one or more positions corresponding to Q15, I21, K26, N44, N47, S53, K57, I68, S83, I104, D108, E109, V116, K131, N135, S140, K142, T157, K166, E170, A173, M195, Q200, M205, N206, C207, N240, R243, and/or K260I in SEQ ID NO: 3.

Optionally, the CjCgtB variant comprises a mutation at one or more positions corresponding to S53 and K166 in SEQ ID NO: 3; a mutation at one or more positions corresponding to K26, S53, and C207 in SEQ ID NO: 3; a mutation at one or more positions corresponding to S53 and C207 in SEQ ID NO: 3; a mutation at one or more positions corresponding to K26, S53, K166, and C207 in SEQ ID NO: 3; a mutation at one or more positions corresponding to S53, K166, and C207 in SEQ ID NO: 3; a mutation at one or more positions corresponding to K26, S53, K166, A173, and C207 in SEQ ID NO: 3; a mutation at one or more positions corresponding to K26, S53, K142, K131, K166, A173, Q200, and C207 in SEQ ID NO: 3; a mutation at one or more positions corresponding to K26, S53, K142, K166, E170, A173, Q200, and C207 in SEQ ID NO: 3; a mutation at one or more positions corresponding to Q15, I21, S53, K57, K142, K166, E170, A173, Q200, M250, and C207 in SEQ ID NO: 3; a mutation at one or more positions corresponding to Q15, K26, S53, K57, K142, K166, I68, N135, E170, A173, Q200, C207, and N240 in SEQ ID NO: 3; a mutation at one or more positions corresponding to K26, N44, S53, K57, I68, N135, K142, K166, E170, A173, Q200, C207, and N240 in SEQ ID NO: 3; a mutation at one or more positions corresponding to Q15, K26, N44, S53, K57, N135, K142, K166, I68, E170, A173, Q200, C207, N240, and K260 in SEQ ID NO: 3; a mutation at one or more positions corresponding to Q15, I21, K26, N44, S53, K57, I68, N135, K142, K166, E170, A173, Q200, C207, N240, and K260 in SEQ ID NO: 3; a mutation at one or more positions corresponding to Q15, I21, K26, N44, S53, K57, I68, I104, N135, K142, K166, E170, A173, Q200, C207, N240, and K260 in SEQ ID NO: 3; a mutation at one or more positions corresponding to I21, K26, S53, K57, I68, N44, I104, N135, D108, K142, K166, E170, A173, Q200, C207, N240, and K260 in SEQ ID NO: 3; a mutation at one or more positions corresponding to I21, N44, N47, S53, K57, I104, D108, N135, S140, K142, K166, I68, E170, A173, Q200, C207, N240, and K260 in SEQ ID NO: 3; a mutation at one or more positions corresponding to I21, N44, N47, S53, K57, I68, I104, D108, V116, N135, S140, K142, K166, E170, A173, Q200, C207, N240, and K260 in SEQ ID NO: 3; a mutation at one or more positions corresponding to I21, N44, N47, S53, K57, I68, I104, D108, V116, N135, S140, K142, K166, E170, A173, Q200, M205, C207, N240, and K260 in SEQ ID NO: 3; a mutation at one or more positions corresponding to Q15, I21, N44, N47, S53, K57, N135, I68, I104, D108, E109, V116, S140, K142, K166, E170, A173, Q200, M205, N206, C207, N240, R243, and K260 in SEQ ID NO: 3; a mutation at one or more positions corresponding to Q15, I21, K26, N44, N47, S53, K57, 168, S83, I104, D108, E109, V116, K131, N135, S140, K142, T157, K166, E170, A173, M195, Q200, M205, N206, C207, N240, R243, and K260 in SEQ ID NO: 3; or a mutation at position K26 in SEQ ID NO: 3.

Optionally, in the CjCgtB variant, the N-terminus of the polypeptide is fused to a maltose binding protein.

Further described herein is a polynucleotide encoding a CjCgtA variant or a CjCgtB variant as described herein. A host cell comprising the polynucleotide is also provided herein. Additionally provided is a reaction mixture comprising a CjCgtA variant or a CjCgtB variant as described herein. The reaction mixture optionally comprises a glycosylation donor comprising a sugar component, a glycosylation acceptor comprising a sphingosine moiety, and/or a detergent (e.g., an anionic detergent or a non-ionic detergent).

Further described herein is a method for preparing a glycosylated molecule, comprising: forming a reaction mixture comprising (i) a glycosylation donor comprising a sugar component; (ii) a glycosylation acceptor comprising a sphingosine moiety; and (iii) a glycosyltransferase, wherein the glycosyltransferase is a CjCgtA variant as described herein or a CjCgtB variant as described herein; and maintaining the reaction mixture under conditions suitable for forming a glycosylated molecule. Optionally, the reaction mixture comprises a detergent. The detergent is optionally an anionic detergent (e.g., sodium cholate) or a non-ionic detergent.

The details of one or more examples are set forth in the drawings and the description below. Other features, objects, and advantages will be apparent from the description and drawings, and from the claims.

### DESCRIPTION OF THE DRAWINGS

FIG. 1 shows the gene (Panel A) and the protein (Panel B) sequences of MBP-Δ15CjCgtA-His₆. The underlined amino acid sequence was from the pMAL-c2X vector and was the linker in the fusion protein.

FIG. 2 shows the gene (Panel A) and the protein (Panel B) sequences of MBP-CjCgtBΔ30-His₆. The underlined amino acid sequence was from the pMAL-c2X vector and was the linker in the fusion protein.

FIG. 3 shows the SDS-PAGE analyses for expression and purification of MBP-Δ15CjCgtA-His₆ (Panel A) and MBP-CjCgtBΔ30-His₆ (Panel B). Lanes: BI, whole cell extract before induction; Al, whole cell extract after induction; L, lysate after induction; PP, Ni²⁺-NTA column purified protein; M, protein markers (Bio-Rad Precision Plus Protein™ Standards, 10-250 kDa).

FIG. 4 shows the pH profiles of MBP-Δ15CjCgtA-His₆ (Panel A) and MBP-CjCgtBΔ30-His₆ (Panel B). Buffers used were: citric acid-sodium citrate, pH 4.0-5.5; PBS, pH 6.0-7.0; Tris-HCl, pH 7.5-8.5; and glycine-NaOH, pH 9.0-11.0.

FIG. 5 shows the effects of divalent metal ions, EDTA, and DTT on the activities of MBP-Δ15CjCgtA-His₆ (Panel A) and MBP-CjCgtBΔ30-His₆ (Panel B).

FIG. 6 shows the thermal stability profiles of MBP-Δ15CjCgtA-His₆ (Panel A) and MBP-CjCgtBΔ30-His₆ (Panel B). The reactions without incubation were used as controls.

FIG. 7 contains ¹H and ¹³C nuclear magnetic resonance (NMR) spectra of Neu5Ac-containing GM1 sphingosine (Neu5Ac-GM1βSph).

FIG. 8 contains ¹H and ¹³C NMR spectra of Neu5Gc-containing GM1 sphingosine (Neu5Gc-GM1βSph).

FIG. 9 contains ¹H and ¹³C NMR of Neu5Ac-containing GM1 (Neu5Ac-GM1).

FIG. 10 contains ¹H and ¹³C NMR of Neu5Gc-containing GM1 (Neu5Gc-GM1).

FIG. 11, Panels A, B, C, and D show schematic examples of syntheses of compounds described herein.

FIG. 12 contains ¹H and ¹³C NMR spectra of GM2βSph (d18:1).

FIG. 13 contains ¹H and ¹³C NMR spectra of GM1βSph (d18:1).

FIG. 14 contains ¹H and ¹³C NMR spectra of GD2βSph (d18:1).

FIG. 15 contains ¹H and ¹³C NMR spectra of GD1bβSph (d18:1).

FIG. 16 contains ¹H and ¹³C NMR spectra of GT2βSph (d18:1).

FIG. 17 contains ¹H and ¹³C NMR spectra of GT1cβSph (d18:1).

FIG. 18 contains ¹H and ¹³C NMR spectra of GA2βSph (d18:1).

FIG. 19 contains ¹H and ¹³C NMR spectra of GA1βSph (d18:1).

FIG. 20 contains ¹H and ¹³C NMR spectra of GM2βSph (d20:1).

FIG. 21 contains ¹H and ¹³C NMR spectra of GM1βSph (d20:1).

FIG. 22 contains ¹H and ¹³C NMR spectra of GD2βSph(d20:1).

FIG. 23 shows SDS-PAGE analysis results for the expression and purification of MBP-Δ15CjCgtA-His₆ D4, D6, D8, and D4-Y238E mutants. Lanes: BI, before induction; Al, after induction; Lys, lysate; E, purified protein; M, PageRuler™ Plus Prestained Protein Ladder, 10 to 250 kDa.

FIG. 24 shows thermal shift assay results for MBP-Δ15CjCgtA-His₆ (WT, Tm=46.1±0.7° C.) and its D4-Y238E mutant (Tm=51.5±0.3° C.).

FIG. 25 shows activity comparison of MBP-Δ15CjCgtA-His₆ (WT) and its D4-Y238E mutant in catalyzing the formation of GM2βSph (d18:1) from GM3βSph (d18:1) and UDP-GalNAc (A) using thin layer chromatography (“TLC”) (B) and high resolution mass spectrometry (“HRMS”) (C) assays. Lanes in B: 1, GM3βSph acceptor substrate standard, 2, WT enzyme reaction mixture; 3, D4-Y238E mutant reaction mixture. Reaction conditions: GM3βSph (d18:1) (10 mM), UDP-GalNAc (15 mM), enzyme (~0.1 mg/mL), MgCl₂ (10 mM), Tris-HCl (pH 7.4, 100 mM), 30° C.

FIG. 26 shows activity comparison of MBP-Δ15CjCgtA-His₆ (WT) and its D4-Y238E mutant via ultra-high-performance liquid chromatography (UHPLC) (Panel A) and HRMS (Panel B). Reaction condition: GM3βNHCbz 1 mM, UDP-GalNAc 1.5 mM, CjCgtA enzyme ~0.1 mg/mL, MgCl₂ (10 mM), Tris-HCl (100 mM, pH 7.4), 30° C., 10 min. Inset: UHPLC peaks of the GM3βNHCbz substrate and the GM2βNHCbz product.

FIG. 27 shows the amino acid sequences of the CjCgtA PROSS mutants, CjCgtA D4 (Panel A), CjCgtA D6 (Panel B), CjCgtA D8 (Panel C), and from the alignment of the amino acid sequences of the wild-type enzyme with the mutants designed using the Protein Repair One Stop Shop (PROSS) (Panel D).

FIG. 28 shows the DNA sequence of gene fragments of CjCgtA D4 (Panel A), CjCgtA D6 (Panel B), and CjCgtA D8 (Panel C).

FIG. 29 shows the DNA sequence of MBP-Δ15CjCgtA-His₆ D4-Y238E mutant. The sequences from the vector and the His₆ tag are underlined. The codon for E238 is bolded and underlined.

FIG. 30 shows the protein sequence of MBP-Δ15CjCgtA-His₆ D4-Y238E mutant. The sequences from the vector and the His₆ tag are underlined. The linker sequences are italicized. E238 is bolded and underlined.

### DETAILED DESCRIPTION

### I. General

Glycosphingolipids are sugar-conjugated lipids that are important to various biological processes including protein sorting, signal transduction, membrane trafficking, viral and bacterial infection, and cell to cell communications. Obtaining pure glycosphingolipids is important to illustrate the biological significance of both the glycan and the lipid (ceramide) portions at the molecular level. Therefore, developing efficient synthetic approaches for these diverse glycosphingolipids is urgently needed. In addition, glycosphingosines are also potential diagnostic and therapeutic tools.

Chemical synthetic methods have been developed for glycosphingolipids in recent years. These methods rely heavily on sophistic chemical synthesis of oligosaccharides with tedious and challenging protection and deprotection process. The yields for these previously developed syntheses were usually low due to the long synthetic schemes involved and several challenging glycosylation reactions.

The glycosyltransferase-based one-pot chemoenzymatic strategy described herein has distinct advantages on obtaining glycosphingolipids. The structurally defined glycosphingosines are produced via one-pot multienzyme (OPME) chemoenzymatic strategy using glycosyl sphingosines as acceptors. The use of glycosyl sphingosine has great advantages in glycolipid synthesis. The technique can be used to couple various glycans to lactosyl sphingosine (LacβSph) via OPME sialylation system to generate complex glycosyl sphingosines, and the latter coupling of fatty acids with amines in the sphingosine chain after glycosylation efficiently introduces different fatty acid structure to the glycosyl sphingosine products. Both glycosphingosine and glycosphingolipid products can be readily purified from reaction mixture by passing through a C18 cartridge.

Also described herein are engineered glycosyltransferases Campylobacter jejuni β1-4GalNAcT (CjCgtA) and Campylobacter jejuni β1-3-galactosyltransferase (CjCgtB) to improve the efficiency of GM1 sphingosine, which can be used for GM1 sphingosine synthesis. The combined well-designed OPME chemoenzymatic strategies and process engineering strategies lead to a multistep one-pot multienzyme (MSOPME) synthetic process for quick access of complex structurally defined glycosyl sphingolipids including GM3, GM2 and GM1. This approach can be readily extended to the synthesis of other glycosphingosines and glycosphingolipids.

To explore their therapeutic potentials, it is essential to obtain structurally defined GM1 gangliosides and GM1 sphingosines in sufficient amounts. Many experiments reported in the literature were performed with GM1 purified from mammalian brains which were mixtures of GM1 molecules containing different sphingosines (e.g. d18:1, d20:1) and various fatty acyl structures including those with varied lengths and different degrees of unsaturation. Pure structurally defined GM1 obtained by synthesis may be used to resolve some of the inconsistent or even controversial results that have been reported and will avoid the concern of using animal brain-derived products as therapeutics. Structurally defined gangliosides are also essential standards for analyzing ganglioside structures and components in tissue samples.

Previously, chemical synthesis of GM1 from ceramide was achieved from its partially protected derivative or a cyclic glucosylceramide intermediate with glycosyl trichloroacetimidate donors. It was also chemically synthesized from a partially protected azido-derivative of sphingosine acceptor and a thioethyl glycosyl donor. Long synthetic schemes with multiple protection and deprotection steps as well as numerous glycosylation and purification processes were involved, which were time consuming and resulted in low yields for total synthesis of GM1.

A chemoenzymatic total synthetic strategy for the formation of GM1 and other glycosphingolipids was previously developed. The method involves chemical synthesis of lactosylsphingosine (LacβSph) as a key intermediate. LacβSph is a water-soluble substrate for glycosyltransferase-based one-pot multienzyme (OPME) reactions for the formation of more complex glycosylsphingosines which are readily converted to the target glycosphingolipids by chemical installation of a desired fatty acyl chain. The product purification of both glycosphingosines and glycosphingolipids is facilitated by their hydrophobic tails, which can be achieved in less than 30 minutes using a simple C18-cartridge purification process.

Two of the glycosyltransferases for the synthesis of GM1 sphingosine, including Campylobacter jejuni β1-4GalNAcT (CjCgtA) and β1-3-galactosyltransferase (CjCgtB), were not stable. In addition, the glycosphingosines were poorer acceptor substrates compared to the corresponding oligosaccharides for these two glycosyltransferases, thus larger amounts of CjCgtA and CjCgtB and a longer reaction time were needed for the production of GM1 sphingosine compared to GM1 oligosaccharide. Furthermore, a product purification process was carried out after every OPME to obtain intermediate glycosylsphingosines (such as GM3 and GM2 sphingosines), which was ideal when all intermediates were targets but the process could be simplified if a glycosphingolipid with a long glycan chain is the desired target.

Described herein is a significantly improved chemoenzymatic total synthesis of GM1 gangliosides containing either the most abundant Neu5Ac or the non-human Neu5Gc sialic acid form. LacβSph is chemically synthesized from a sphingosine glycosyl acceptor obtained by a four-step process, a much shorter route than the ones that we reported previously. Both CjCgtA and CjCgtB are engineered to improve their expression levels and stability. As described herein, GM1 sphingosines containing Neu5Ac (in gram-scale) and Neu5Gc are synthesized from LacβSph using a streamlined multistep sequential OPME (MSOPME) process without the isolation of intermediate glycosphingosines. The addition of a detergent (e.g., sodium cholate), improves the efficiency of the last two OPME steps for the GM1 sphingosine synthesis, leading to shorter reaction times and the need for lower amounts of CjCgtA and CjCgtB. These developments, as described herein, pave the way for large-scale production of GM1 sphingosine and GM1 ganglioside in a time-efficient manner.

### II. Definitions

The terms “peptide,” “polypeptide,” and “protein” are used interchangeably herein to refer to a polymer of amino acid residues. All three terms apply to naturally occurring amino acid polymers and non-natural amino acid polymers, as well as to amino acid polymers in which one (or more) amino acid residue is an artificial chemical mimetic of a corresponding naturally occurring amino acid. As used herein, the terms encompass amino acid chains of any length, including full-length proteins, wherein the amino acid residues are linked by covalent peptide bonds.

The terms “mutant” and “variant,” in the context of glycosyltransferases described herein, mean a polypeptide, typically recombinant, that comprises one or more amino acid substitutions relative to a corresponding, naturally-occurring or unmodified glycosyltransferase.

The term “amino acid” refers to any monomeric unit that can be incorporated into a peptide, polypeptide, or protein. Amino acids include naturally-occurring a-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally-occurring amino acids are those encoded by the genetic code, as well as those amino acids that are later modified, e.g., hydroxyproline, y-carboxyglutamate and O-phosphoserine. Naturally-occurring a-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring a-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid.

Amino acids may be referred to herein by either the commonly known three letter symbols or by the one-letter symbols recommended by the IUPAC-IUB Biochemical Nomenclature Commission. Nucleotides, as described herein, may also be referred to by their commonly accepted single-letter codes.

With respect to amino acid sequences, one of skill in the art will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acid includes, without limitation, a naturally-occurring amino acid such as an L-amino acid, a stereoisomer of a naturally occurring amino acid such as a D-amino acid, and an unnatural amino acid such as an amino acid analog, amino acid mimetic, synthetic amino acid, N-substituted glycine, and N-methyl amino acid.

The terms “amino acid modification” and “amino acid alteration” refer to a substitution, a deletion, or an insertion of one or more amino acids. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some examples, an amino acid with an acidic side chain, e.g., E or D, may be substituted with its uncharged counterpart, e.g., Q or N, respectively; or vice versa. Each of the following eight groups contains exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “nucleic acid,” “nucleotide,” and “polynucleotide” refer to deoxyribonucleic acids (DNA) or ribonucleic acids (RNA) and polymers. The term includes, but is not limited to, single-, double-, or multi-stranded DNA or RNA, genomic DNA, cDNA, and DNA-RNA hybrids, as well as other polymers comprising purine and/or pyrimidine bases or other natural, chemically modified, biochemically modified, non-natural, synthetic, or derivatized nucleotide bases. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), orthologs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “nucleotide sequence encoding a peptide” and “gene” refer to the segment of DNA involved in producing a peptide chain. In addition, a gene will generally include regions preceding and following the coding region (leader and trailer) involved in the transcription/translation of the gene product and the regulation of the transcription/translation. A gene can also include intervening sequences (introns) between individual coding segments (exons). Leaders, trailers, and introns can include regulatory elements that are necessary during the transcription and the translation of a gene (e.g., promoters, terminators, translational regulatory sequences such as ribosome binding sites and internal ribosome entry sites, enhancers, silencers, insulators, boundary elements, replication origins, matrix attachment sites and locus control regions, etc.). A “gene product” can refer to either the mRNA or protein expressed from a particular gene.

“Percentage of sequence identity” is determined by comparing two optimally aligned sequences over a comparison window, wherein the portion of the sequence (e.g., a peptide as described herein) in the comparison window may comprise additions or deletions (i.e., gaps) as compared to the reference sequence which does not comprise additions or deletions, for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity.

“Identical” and “identity,” in the context of two or more nucleic acids or polypeptide sequences, refer to two or more sequences or subsequences that are the same. Sequences are “substantially identical” to each other if they have a specified percentage of nucleotides or amino acid residues that are the same (e.g., at least 75%, at least 80%, at least 85%, at least 90%, at least 95%, or at least 99% identical over a specified region), when compared and aligned for maximum correspondence over a comparison window, or designated region as measured using one of the following sequence comparison algorithms or by manual alignment and visual inspection. These definitions also refer to the complement of a nucleic acid test sequence.

“Similarity” and “percent similarity,” in the context of two or more polypeptide sequences, refer to two or more sequences or subsequences that have a specified percentage of amino acid residues that are either the same or similar as defined by a conservative amino acid substitutions (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% similar over a specified region), when compared and aligned for maximum correspondence over a comparison window, or designated region as measured using one of the following sequence comparison algorithms or by manual alignment and visual inspection. Sequences are “substantially similar” to each other if, for example, they are at least 20%, at least 25%, at least 30%, at least 35%, at least 40%, at least 45%, at least 50%, or at least 55% similar to each other.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

Methods of alignment of sequences for comparison are well-known in the art. Optimal alignment of sequences for comparison can be conducted, which are useful for determining percent sequence identity and sequence similarity, are the BLAST and BLAST 2.0 algorithms, which are described in Altschul et al., (1990)J Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see, e.g., Henikoff and Henikoff, Proc. Natl. Acad. Sci. USA 89:10915 (1989)).

The BLAST algorithm also performs a statistical analysis of the similarity between 30 two sequences (see, e.g., Karlin and Altschul, Proc. Natl. Acad. Sci. USA, 90: 5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or peptides are substantially identical is that the peptide encoded by the first nucleic acid is immunologically cross reactive with the antibodies raised against the peptide encoded by the second nucleic acid. Thus, a peptide is typically substantially identical to a second peptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The terms “transfection” and “transfected” refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88.

The terms “expression” and “expressed” in the context of a gene refer to the transcriptional and/or translational product of the gene. The level of expression of a DNA molecule in a cell may be determined on the basis of either the amount of corresponding mRNA that is present within the cell or the amount of protein encoded by that DNA produced by the cell.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “promoter,” as used herein, refers to a polynucleotide sequence capable of driving transcription of a coding sequence in a cell. Thus, promoters used in the polynucleotide constructs described herein include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “constitutive promoter” is one that is capable of initiating transcription in nearly all tissue types, whereas a “tissue-specific promoter” initiates transcription only in one or a few particular tissue types. An “inducible promoter” is one that initiates transcription only under particular environmental conditions or developmental conditions.

A polynucleotide/polypeptide sequence is “heterologous” to an organism or a second polynucleotide/polypeptide sequence if it originates from a different species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., is a genetically engineered coding sequence, e.g., from a different gene in the same species, or an allele from a different ecotype or variety).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. For example, recombinant cells express genes that are not found within the native (non-recombinant) form of the cell or express native genes that are otherwise abnormally expressed, under-expressed, or not expressed at all.

An “expression cassette” refers to a nucleic acid construct, which when introduced into a host cell, results in transcription and/or translation of a RNA or polypeptide, respectively. Antisense constructs or sense constructs that are not or cannot be translated are expressly included by this definition. One of skill will recognize that the inserted polynucleotide sequence need not be identical, but may be only substantially similar to a sequence of the gene from which it was derived.

The terms “vector” and “recombinant expression vector” refer to a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain.

As used herein, the term “glycosyltransferase” refers to a polypeptide that catalyzes the formation of an oligosaccharide from a nucleotide-sugar an acceptor sugar. Nucleotide-sugars include, but are not limited to, nucleotide diphosphate sugars (NDP-sugars) and nucleotide monophosphate sugars (NMP-sugars) such as a cytidine monophosphate sugar (CMP-sugar). In general, a glycosyltransferase catalyzes the transfer of the monosaccharide moiety of an NDP-sugar or CMP-sugar to a hydroxyl group of the acceptor sugar. The covalent linkage between the monosaccharide and the acceptor sugar can be a 1-3 linkage, a 1-4 linkage, a 1-6-linkage, a 1-2 linkage, a 2-3 linkage, a 2-6 linkage, a 2-8 linkage, or a 2-9 linkage as described above. The linkage may be in the α- or β-configuration with respect to the anomeric carbon of the monosaccharide. Other types of linkages may be formed by the glycosyltransferases in the methods described herein. Glycosyltransferases include, but are not limited to, heparosan synthases (HSs), glucosaminyltransferases, N-acetylglucosaminyltransferases, glucosyltransferasess, glucuronyltransferases, and sialyltransferases.

As used herein, the term “oligosaccharide” refers to a compound containing at least two monosaccharides covalently linked together. Oligosaccharides include disaccharides, trisaccharides, tetrasaccharides, pentasaccharides, hexasaccharides, heptasaccharides, octasaccharides, and the like. Covalent linkages generally consist of glycosidic linkages (i.e., C—O—C bonds) formed from the hydroxyl groups of adjacent sugars. Linkages can occur between the 1-carbon and the 4-carbon of adjacent sugars (i.e., a 1-4 linkage), the 1-carbon and the 3-carbon of adjacent sugars (i.e., a 1-3 linkage), the 1-carbon and the 6-carbon of adjacent sugars (i.e., a 1-6 linkage), or the 1-carbon and the 2-carbon of adjacent sugars (i.e., a 1-2 linkage). Linkages can occur between the 2-carbon and the 3-carbon of adjacent sugars (i.e., a 2-3 linkage), the 2-carbon and the 6-carbon of adjacent sugars (i.e., a 2-6 linkage), the 2-carbon and the 8-carbon of adjacent sugars (i.e., a 2-8 linkage), or the 2-carbon and the 9-carbon of adjacent sugars (i.e., a 2-9 linkage). A sugar can be linked within an oligosaccharide such that the anomeric carbon is in the α- or β-configuration. The oligosaccharides prepared according to the methods described herein can also include linkages between carbon atoms other than the 1-, 2-, 3-, 4-, and 6-carbons or the 2-, 3-, 6-, 8-, and 9-carbons.

“Acceptor glycoside” or “glycosylation acceptor” refers to a substance (e.g., a glycosylated amino acid, a glycosylated protein, an oligosaccharide, or a polysaccharide) containing a sphingosine moiety that accepts a sugar moiety from a donor substrate.

As used herein, the term “kinase” refers to a polypeptide that catalyzes the covalent addition of a phosphate group to a substrate. The substrate for a kinase used in the methods described herein is generally a sugar as defined above, and a phosphate group is added to the anomeric carbon (i.e. the “1” position) of the sugar. The product of the reaction is a sugar-1-phosphate. Kinases include, but are not limited to, N-acetylhexosamine 1-kinases (NahKs), glucuronokinases (GlcAKs), glucokinases (GlcKs), galactokinases (GalKs), monosaccharide-1-kinases, and xylulokinases. Certain kinases utilize nucleotide triphosphates, including adenosine-5′-triphosphate (ATP) as substrates.

As used herein, the term “dehydrogenase” refers to a polypeptide that catalyzes the oxidation of a primary alcohol. In general, the dehyrogenases used in the methods described herein convert the hydroxymethyl group of a hexose (i.e. the C6-OH moiety) to a carboxylic acid. Dehydrogenases useful in the methods described herein include, but are not limited to, UDP-glucose dehydrogenases (Ugds).

As used herein, the term “nucleotide-sugar pyrophosphorylase” refers to a polypeptide that catalyzes the conversion of a sugar-i-phosphate to a UDP-sugar. In general, a uridine-5′-monophosphate moiety is transferred from uridine-5′-triphosphate to the sugar-i-phosphate to form the UDP-sugar. Examples of nucleotide-sugar pyrophosphorylases include glucosamine uridylyltransferases (GlmUs) and glucose-l-phosphate uridylyltransferases (GalUs). Nucleotide-sugar pyrophosphorylases also include promiscuous UDP-sugar pyrophosphorylases, termed “USPs,” that can catalyze the conversion of various sugar-i-phosphates to UDP-sugars including UDP-Glc, UDP-GlcNAc, UDP-GlcNH₂, UDP-Gal, UDP-GalNAc, UDP-GalNH₂, UDP-Man, UDP-ManNAc, UDP-ManNH₂, UDP-GlcA, UDP-IdoA, UDP-GalA, and their substituted analogs.

As used herein, the term “pyrophosphatase” (abbreviated as PpA) refers to a polypeptide that catalyzes the conversion of pyrophosphate (i.e., P₂O₇⁴⁻, HP₂O₇³⁻, H₂P₂O₇²⁻, H₃P₂O₇⁻) to two molar equivalents of inorganic phosphate (i.e., PO₄³⁻, HPO₄²⁻, H₂PO₄⁻).

An amino acid residue “corresponding to an amino acid residue [X] in [specified sequence,” or an amino acid substitution “corresponding to an amino acid substitution [X] in [specified sequence]” refers to an amino acid in a polypeptide of interest that aligns with the equivalent amino acid of a specified sequence. Generally, as described herein, the amino acid corresponding to a position of a specified polypeptide sequence can be determined using an alignment algorithm such as BLAST.

### III. Campylobacter jejuni β1-4GalNAcT (CjCgtA) variants

Described herein are Campylobacter jejuni P1-4GalNAcT (CjCgtA) variants exhibiting improved stability and increased glycosylation efficiency as compared to the wildtype CjCgtA enzyme. The CjCgtA variant includes a polypeptide having at least 80% identity to the amino acid sequence set forth in SEQ ID NO: 1, SEQ ID NO: 2, or SEQ ID NO: 23. For example, the CjCgtA variants as described herein can include a polypeptide having a percent sequence identity to SEQ ID NO: 1, SEQ ID NO: 2, or SEQ ID NO: 23 of at least 20, 30, 40, 50, 55, 60, 65, 70, 75, 80, 85, 90, 91, 92, 93, 94, 95, 96, 97, 98 or at least 99%. In some examples, percent sequence identity can be at least 80%. In some examples, percent sequence identity can be at least 90%. In some examples, percent sequence identity can be at least 95%. In some examples, the CjCgtA variant has a sequence identity of at least about 90%, 91%, 92%, 93%, 94%, 95%, 96%, 97%, 98% or 99%, or is identical to SEQ ID NO: 1, SEQ ID NO: 2, or SEQ ID NO: 23. Also described herein is an isolated or purified polypeptide including an amino acid sequence selected from SEQ ID NO:1, SEQ ID NO: 2, or SEQ ID NO: 23.

The precise length of the CjCgtA variants can vary, and certain variants can be advantageous for expression and purification of the enzymes with high yields. For example, removal of certain peptide subunits from the overall polypeptide sequence of CjCgtA can improve solubility of the enzyme and increase expression levels. Alternatively, addition of certain peptide or protein subunits to a CjCgtA polypeptide sequence can modulate expression, solubility, activity, or other properties. The CjCgtA variants described herein can include point mutations at any position of the CjCgtA sequence (e.g., SEQ ID NO: 1 or SEQ ID NO: 2). The mutants can include any suitable amino acid other than the native amino acid. For example, the amino acid can be V, I, L, M, F, W, P, S, T, A, G, C, Y, N, Q, D, E, K, R, or H. Amino acid and nucleic acid sequence alignment programs are readily available (see, e.g., those referred to supra) and, given the particular motifs identified herein, serve to assist in the identification of the exact amino acids (and corresponding codons) for modification in accordance with the present description.

In some examples, the polypeptide further comprises one or more heterologous amino acid sequences located at the N-terminus and/or the C-terminus of the polypeptide. The polypeptide can contain a number of heterologous sequences that are useful for expressing, purifying, and/or using the polypeptide. The polypeptide can contain, for example, a poly-histidine tag (e.g., a His₆ tag); a calmodulin-binding peptide (CBP) tag; a NorpA peptide tag; a Strep tag (e.g., Trp-Ser-His-Pro-Gln-Phe-Glu-Lys) for recognition by/binding to streptavidin or a variant thereof, a FLAG peptide (i.e., Asp-Tyr-Lys-Asp-Asp-Asp-Asp-Lys) for recognition by/binding to anti-FLAG antibodies (e.g., M1, M2, M5); a glutathione S-transferase (GST); or a maltose binding protein (MBP) polypeptide. In some examples, described herein is an isolated polypeptide comprising the amino acid sequence set forth in SEQ ID NO: 1, SEQ ID NO: 2, or SEQ ID NO: 23 with a His₆ peptide fused to the C-terminal residue of the amino acid sequence. In some examples, the polypeptide comprises the amino acid sequence set forth in SEQ ID NO: 1, SEQ ID NO: 2, or SEQ ID NO: 23 with a His₆ peptide fused to the C-terminal residue of the amino acid sequence. In some examples, described herein is an isolated polypeptide comprising the amino acid sequence set forth in SEQ ID NO: 1, SEQ ID NO: 2, or SEQ ID NO: 23 with an MBP polypeptide fused to the N-terminal residue of the amino acid sequence. In some examples, the polypeptide comprises the amino acid sequence set forth in SEQ ID NO: 1, SEQ ID NO: 2, or SEQ ID NO: 23 with an MBP polypeptide fused to the N-terminal residue of the amino acid sequence.

Optionally, the CjCgtA variant comprises a mutation at one or more positions corresponding to G58, A110, and R166 in SEQ ID NO: 1. The CjCgtA variant can further comprise a mutation at P301 in SEQ ID NO: 1; a mutation at one or more positions corresponding to L97 and Q244 in SEQ ID NO: 1; a mutation at one or more positions corresponding to L142 and K229 in SEQ ID NO: 1; a mutation at N107 in SEQ ID NO: 1; a mutation at V50 in SEQ ID NO: 1; a mutation at one or more positions corresponding to Q169, S212, S213, and A282 in SEQ ID NO: 1; a mutation at G200 in SEQ ID NO: 1; a mutation at one or more positions corresponding to D 118, K286, and A296 in SEQ ID NO: 1; a mutation at S287 in SEQ ID NO: 1; a mutation at S243 in SEQ ID NO: 1; a mutation at S193 in SEQ ID NO: 1; a mutation at N124 in SEQ ID NO: 1; a mutation at L80 in SEQ ID NO: 1; a mutation at K46 in SEQ ID NO: 1; a mutation at K288 in SEQ ID NO:1; a mutation at K35 in SEQ ID NO: 1; a mutation at one or more positions corresponding to E170, F214, and I215 in SEQ ID NO: 1; and/or a mutation at one or more positions corresponding to Kill, S131, V190, R209, R210, V246, E289, and E304 in SEQ ID NO: 1. Optionally, the CjCgtA variant comprises a mutation at one or more positions corresponding to K35, K46, V50, G58, L80, L97, N107, A110, K111, D118, N124, S131, L142, R166, Q169, E170, V790, S193, G200, R209, R210, S212, S213, F214, L215, R218, K229, L23, S243, Q244, V246, A282, K286, S287, K288, E289, A296, P301, and E304 in SEQ ID NO: 1.

Exemplary CjCgtA variants of SEQ ID NO: 1 as described herein are outlined below in Table 1.

TABLE 1

| Design Number | Mutations within SEQ ID NO: 1 |
|---|---|
| 1 | G58A, A110P, R166L |
| 2 | G58A, A110P, R166L, P301E |
| 3 | G58A, L97I, A110P, R166L, Q244K, P301E |
| 4 | G58A, L97I, A110P, L142Q, R166L, S213F, Q244K, P301E |
| 5 | G58A, L97I, N107S, A110P, L142Q, R166M, P301E, Q244K |
| 6 | G58A, L97I, N107S, A110P, L142Q, R166L, Q169N, S2121, S213F, |
| | A282R, Q244K, P301E |
| 7 | G58A, L97I, N107S, A110P, L142Q, R166L, Q169N, G200D, S212I, |
| | A282R, Q244K, P301E |
| 8 | V50K, G58A, L97I, N107S, A110P, D118Q, L142Q, R166L, Q169N, |
| | G200D, S212I, Q244K, A282R, K286H, A296S, P301E |
| 9 | V50K, G58A, L97I, N107S, A110P, D118Q, L142Q, R166L, Q169N, |
| | G200D, S212I, Q244K, A282R, K286H, S287H, A296S, P301E |
| 10 | V50K, G58A, L97I, N107S, A110P, D118Q, L142Q, R166L, Q169N, |
| | G200D, S212I, Q244K, S243A, A282R, K286H, S287H, A296S, |
| | P301E |
| 11 | V50K, G58A, L97I, N107S, A110P, D118Q, L142Q, R166L, Q169N, |
| | G200D, S212I, S213K, S243A, Q244K, A282R, K286H, S287H, |
| | A296S, P301E |
| 12 | V50K, G58A, L97I, N107S, A110P, D118Q, L142Q, R166L, Q169N, |
| | S193I, G200D, S212I, S213K, Q244K, A282R, K286H, S287H, |
| | A296S, S243A, P301E |
| 13 | V50K, G58A, L97I, N107S, A110P, D118Q, N124S, L142Q, R166L, |
| | Q169N, S193I, G200D, S212I, S213K, S243A, Q244K, A282R, |
| | K286H, S287H, A296S, P301E |
| 14 | V50K, G58A, L80I, L97I, N107S, A110P, D118Q, N124S, L142Q, |
| | R166L, Q169N, S193I, G200D, S212I, S213K, S243A, Q244K, |
| | A282R, K286H, S287H, A296S, P301E |
| 15 | K46R, V50K, G58A, L80I, L97I, N107S, A110P, D118Q, N124S, |
| | L142Q, R166L, Q169N, S193I, G200D, S212I, S213K, S243A, |
| | Q244K, A282R, K286H, S287H, A296S, P301E |
| 16 | K46R, V50K, G58A, L80I, L97I, N107S, A110P, D118Q, N124S, |
| | L142Q, R166L, Q169N, S1931, G200D, S212I, S213K, S243A, |
| | Q244K, A282R, K286H, S287H, K288R, A296S, P301E |
| 17 | K35E, K46R, V50K, G58A, L80I, L97I, N107S, A110P, D118Q, |
| | N124S, L142Q, R166L, Q169N, S193I, G200D, S212I, S213K, |
| | S243A, Q244K, A282R, K286H, S287H, K288R, A296S, P301E |
| 18 | K35E, K46R, V50K, G58A, L80I, L97I, N107S, A110P, D118Q, |
| | N124S, L142Q, R166M, Q169N, E170D, S193I, G200D, S212I, |
| | S213K, F214W, I215V, S243A, A282R, K286H, S287H, K288R, |
| | A296S, P301E |
| 19 | K35E, K46R, V50K, G58A, L80I, L97I, N107S, A110P, D118Q, |
| | N124S, L142Q, R166L, Q169N, E170D, S193I, G200D, S212I, |
| | S213K, F214W, I215V, S243A, Q244K, A282R, K286H, S287H, |
| | K288R, A296S, P301E |
| 20 | K35E, K46R, V50K, G58A, L80I, L97I, N107S, A110P, D118Q, |
| | N124S, L142Q, R166M, Q169N, E170D, S193I, G200D, S212I, |
| | S213K, F214W, 1215V, Q244K, S243A, A282R, K286H, S287H, |
| | K288R, A296S, P301E |
| 21 | K35E, K46R, V50P, G58A, L80I, L97I, N107S, A110P, K111V, |
| | D118Q, N124S, S131Y, L142Q, R166L, Q169N, E170D, V190I, |
| | S193I, G200D, R209N, R210K, S212I, S213K, F214W, I215V, |
| | S243A, 4Q244K, V246I, A282R, K286H, S287N, K288R, E289L, |
| | A296S, P301E, E304Y |
| 22 | V50P, G58A, L97I, N107S, A110P, L142Q, R166M, K229T, Q244K, |
| | P301E |
| 23 | V50P, G58A, L97I, N107S, A110P, K111V, L142Q, R166M, R218M, |
| | K229T, Q244K, A282R, P301E |
| 24 | V50P, G58A, L80I, L97I, N107S, A110P, K111V, A136V, L142Q, |
| | R166M, R209N, S213K, I215V, R218M, K229T, L231T, Q244K, |
| | A282R, A296S, P301E |

Optionally, the CjCgtA variant comprises a mutation at one or more positions corresponding to G43, A95, and R151 in SEQ ID NO: 2. The CjCgtA variant can further comprise a mutation at P286 in SEQ ID NO: 2; a mutation at one or more positions corresponding to L82 and Q229 in SEQ ID NO: 2; a mutation at one or more positions corresponding to L127 and K214 in SEQ ID NO: 2; a mutation at N92 in SEQ ID NO: 2; a mutation at V35 in SEQ ID NO: 2; a mutation at one or more positions corresponding to Q154, S197, S198, and A267 in SEQ ID NO: 2; a mutation at G185 in SEQ ID NO: 2; a mutation at one or more positions corresponding to D103, K271, and A281 in SEQ ID NO: 2; a mutation at S272 in SEQ ID NO: 2; a mutation at S228 in SEQ ID NO: 2; a mutation at S178 in SEQ ID NO: 2; a mutation at N109 in SEQ ID NO: 2; a mutation at L65 in SEQ ID NO: 2; a mutation at K31 in SEQ ID NO: 2; a mutation at K273 in SEQ ID NO: 2; a mutation at K20 in SEQ ID NO: 2; a mutation at one or more positions corresponding to E155, F199, and 1200 in SEQ ID NO: 2; and/or a mutation at one or more positions corresponding to K96, S116, V175, R194, R195, V231, E274, and E289 in SEQ ID NO: 2. Optionally, the CjCgtA variant comprises a mutation at one or more positions corresponding to K20, K31, V35, G43, L65, L82, N92, A95, K96, D103, N109, S116, L127, R151, Q154, E155, V175, S178, G185, R194, R195, S197, S198, F199, I200, R203, K214, L216, S228, Q229, V231, A267, K271, S272, K273, E274, A281, P286, and E289 in SEQ ID NO: 2.

Exemplary CjCgtA variants of SEQ ID NO: 2 as described herein are outlined below in Table 2.

TABLE 2

| Design Number | Mutations within SEQ ID NO: 2 |
|---|---|
| 25 | G43A, A95P, R151L |
| 26 | G43A, A95P, R151L, P286E |
| 27 | G43A, L821, A95P, R151L, Q229K, P286E |
| 28 | G43A, L82I, A95P, L127Q, R151L, S198F, Q229K, P286E |
| 29 | G43A, L82I, N92S, A95P, L127Q, R151M, P286E, Q229K |
| 30 | G43A, L821, N92S, A95P, L127Q, R151L, Q154N, S1971, S198F, |
| | A267R, Q229K, P286E |
| 31 | G43A, L82I, N92S, A95P, L127Q, R151L, Q154N, G185D, S197I, |
| | A267R, Q229K, P286E |
| 32 | V35K, G43A, L82I, N92S, A95P, D103Q, L127Q, R151L, Q154N, |
| | G185D, S197I, Q229K, A267R, K271H, A281S, P286E |
| 33 | V35K, G43A, L821, N92S, A95P, D103Q, L127Q, R151L, Q154N, |
| | G185D, S197I, Q229K, A267R, K271H, S272H, A281S, P286E |
| 34 | V35K, G43A, L82I, N92S, A95P, D103Q, L127Q, R151L, Q154N, |
| | G185D, S197I, Q229K, S228A, A267R, K271H, S272H, A281S, |
| | P286E |
| 35 | V35K, G43A, L821, N92S, A95P, D103Q, L127Q, R151L, Q154N, |
| | G185D, S197I, S198K, S228A, Q229K, A267R, K271H, S272H, |
| | A281S, P286E |
| 36 | V35K, G43A, L821, N92S, A95P, D103Q, L127Q, R151L, Q154N, |
| | S178I, G185D, S197I, S198K, Q229K, A267R, K271H, S272H, |
| | A281S, S228A, P286E |
| 37 | V35K, G43A, L82I, N92S, A95P, D103Q, N109S, L127Q, R151L, |
| | Q154N, S178I, G185D, S197I, S198K, S228A, Q229K, A267R, |
| | K271H, S272H, A281S, P286E |
| 38 | V35K, G43A, L65I, L82I, N92S, A95P, D103Q, N109S, L127Q, |
| | R151L, Q154N, S178I, G185D, S197I, S198K, S228A, Q229K, |
| | A267R, K271H, S272H, A281S, P286E |
| 39 | K31R, V35K, G43A, L65I, L82I, N92S, A95P, D103Q, N109S, |
| | L127Q, R151L, Q154N, S178I, G185D, S197I, S198K, S228A, |
| | Q229K, A267R, K271H, S272H, A281S, P286E |
| 40 | K31R, V35K, G43A, L65I, L821, N92S, A95P, D103Q, N109S, |
| | L127Q, R151L, Q154N, S178I, G185D, S197I, S198K, S228A, |
| | Q229K, A267R, K271H, S272H, K273R, A281S, P286E |
| 41 | K20E, K31R, V35K, G43A, L65I, L821, N92S, A95P, D103Q, N109S, |
| | L127Q, R151L, Q154N, S178I, G185D, S197I, S198K, S228A, |
| | Q229K, A267R, K271H, S272H, K273R, A281S, P286E |
| 42 | K20E, K31R, V35K, G43A, L65I, L821, N92S, A95P, D103Q, N109S, |
| | L127Q, R151M, Q154N, E155D, S178I, G185D, S197I, S198K, |
| | F199W, I200V, S228A, A267R, K271H, S272H, K273R, A281S, |
| | P286E |
| 43 | K20E, K31R, V35K, G43A, L65I, L821, N92S, A95P, D103Q, N109S, |
| | L127Q, R151L, Q154N, E155D, S178I, G185D, S197I, S198K, |
| | F199W, I200V, S228A, Q229K, A267R, K271H, S272H, K273R, |
| | A281S, P286E |
| 44 | K20E, K31R, V35K, G43A, L65I, L82I, N92S, A95P, D103Q, N109S, |
| | L127Q, R151M, Q154N, E155D, S178I, G185D, S197I, S198K, |
| | F199W, I200V, Q229K, S228A, A267R, K271H, S272H, K273R, |
| | A281S, P286E |
| 45 | K20E, K31R, V35P, G43A, L65I, L82I, N92S, A95P, K96V, D103Q, |
| | N109S, S116Y, L127Q, R151L, Q154N, E155D, V175I, S178I, |
| | G185D, R194N, R195K, S197I, S198K, F199W, I200V, S228A, |
| | Q229K, V231I, A267R, K271H, S272N, K273R, E274L, A281S, |
| | P286E, E289Y |
| 46 | V35P, G43A, L821, N92S, A95P, L127Q, R151M, K214T, Q229K, |
| | P286E |
| 47 | V35P, G43A, L821, N92S, A95P, K96V, L127Q, R151M, R213M, |
| | K214T, Q229K, A267R, P286E |
| 48 | V35P, G43A, L65I, L821, N92S, A95P, K96V, A121V, L127Q, |
| | R151M, R194N, S198K, I200V, R203M, K214T, L216T, Q229K, |
| | A267R, A281S, P286E |

### IV. Campylobacter jejuni β1-3-Galactosyltransferase (CjCgtB) Variants

Described herein are Campylobacter jejuni β1-3-gal actosyltransferase (CjCgtB) variants exhibiting improved stability and increased glycosylation efficiency as compared to the wildtype CjCgtB enzyme. The CjCgtB variant includes a polypeptide having at least 8000 identity to the amino acid sequence set forth in SEQ ID NO: 3. For example, the CjCgtB variants as described herein can include a polypeptide having a percent sequence identity to SEQ ID NO: 3 of at least 20, 30, 40, 50, 55, 60, 65, 70, 75, 80, 85, 90, 91, 92, 93, 94, 95, 96, 97, 98 or at least 99%. In some examples, percent sequence identity can be at least 80%. In some examples, percent sequence identity can be at least 90%. In some examples, percent sequence identity can be at least 95%. In some examples, the CjCgtB variant has a sequence identity of at least about 90%, 91%, 92%, 93%, 94%, 95%, 96%, 97%, 98% or 99%, or is identical to SEQ ID NO: 3. Also described herein is an isolated or purified polypeptide including an amino acid sequence according to SEQ ID NO:3.

The precise length of the CjCgtB variants can vary, and certain variants can be advantageous for expression and purification of the enzymes with high yields. For example, removal of certain peptide subunits from the overall polypeptide sequence of CjCgtB can improve solubility of the enzyme and increase expression levels. Alternatively, addition of certain peptide or protein subunits to a CjCgtB polypeptide sequence can modulate expression, solubility, activity, or other properties. The CjCgtB variants described herein can include point mutations at any position of the CjCgtB sequence (e.g., SEQ ID NO: 3). The mutants can include any suitable amino acid other than the native amino acid. For example, the amino acid can be V, I, L, M, F, W, P, S, T, A, G, C, Y, N, Q, D, E, K, R, or H. Amino acid and nucleic acid sequence alignment programs are readily available (see, e.g., those referred to supra) and, given the particular motifs identified herein, serve to assist in the identification of the exact amino acids (and corresponding codons) for modification in accordance with the present description.

In some examples, the polypeptide further comprises one or more heterologous amino acid sequences located at the N-terminus and/or the C-terminus of the polypeptide. The polypeptide can contain a number of heterologous sequences that are useful for expressing, purifying, and/or using the polypeptide. The polypeptide can contain, for example, a poly-histidine tag (e.g., a His₆ tag); a calmodulin-binding peptide (CBP) tag; a NorpA peptide tag; a Strep tag (e.g., Trp-Ser-His-Pro-Gln-Phe-Glu-Lys) for recognition by/binding to streptavidin or a variant thereof, a FLAG peptide (i.e., Asp-Tyr-Lys-Asp-Asp-Asp-Asp-Lys) for recognition by/binding to anti-FLAG antibodies (e.g., M1, M2, M5); a glutathione S-transferase (GST); or a maltose binding protein (MBP) polypeptide. In some examples, described herein is an isolated polypeptide comprising the amino acid sequence set forth in SEQ ID NO: 3 with a His₆ peptide fused to the C-terminal residue of the amino acid sequence. In some examples, the polypeptide comprises the amino acid sequence set forth in SEQ ID NO: 3 with a His₆ peptide fused to the C-terminal residue of the amino acid sequence. In some examples, described herein is an isolated polypeptide comprising the amino acid sequence set forth in SEQ ID NO: 3 with an MBP polypeptide fused to the N-terminal residue of the amino acid sequence. In some examples, the polypeptide comprises the amino acid sequence set forth in SEQ ID NO: 3 with an MBP polypeptide fused to the N-terminal residue of the amino acid sequence.

Optionally, the CjCgtB variant comprises a mutation at one or more positions corresponding to K26, S53, and K166 in SEQ ID NO: 3. The CjCgtB variant can further comprise a mutation at one or more positions corresponding to Q15, I21, K26, N44, N47, S53, K57, I68, S83, I104, D108, E109, V116, K131, N135, S140, K142, T157, K166, E170, A173, M195, Q200, M205, N206, C207, N240, R243, and K260I in SEQ ID NO: 3.

Optionally, the CjCgtB variant comprises a mutation at one or more positions corresponding to S53 and K166 in SEQ ID NO: 3; a mutation at one or more positions corresponding to K26, S53, and C207 in SEQ ID NO: 3; a mutation at one or more positions corresponding to S53 and C207 in SEQ ID NO: 3; a mutation at one or more positions corresponding to K26, S53, K166, and C207 in SEQ ID NO: 3; a mutation at one or more positions corresponding to S53, K166, and C207 in SEQ ID NO: 3; a mutation at one or more positions corresponding to K26, S53, K166, A173, and C207 in SEQ ID NO: 3; a mutation at one or more positions corresponding to K26, S53, K142, K131, K166, A173, Q200, and C207 in SEQ ID NO: 3; a mutation at one or more positions corresponding to K26, S53, K142, K166, E170, A173, Q200, and C207 in SEQ ID NO: 3; a mutation at one or more positions corresponding to Q15, I21, S53, K57, K142, K166, E170, A173, Q200, M250, and C207 in SEQ ID NO: 3; a mutation at one or more positions corresponding to Q15, K26, S53, K57, K142, K166, I68, N135, E170, A173, Q200, C207, and N240 in SEQ ID NO: 3; a mutation at one or more positions corresponding to K26, N44, S53, K57, I68, N135, K142, K166, E170, A173, Q200, C207, and N240 in SEQ ID NO: 3; a mutation at one or more positions corresponding to Q15, K26, N44, S53, K57, N135, K142, K166, I68, E170, A173, Q200, C207, N240, and K260 in SEQ ID NO: 3; a mutation at one or more positions corresponding to Q15, I21, K26, N44, S53, K57, I68, N135, K142, K166, E170, A173, Q200, C207, N240, and K260 in SEQ ID NO: 3; a mutation at one or more positions corresponding to Q15, I21, K26, N44, S53, K57, I68, I104, N135, K142, K166, E170, A173, Q200, C207, N240, and K260 in SEQ ID NO: 3; a mutation at one or more positions corresponding to I21, K26, S53, K57, I68, N44, I104, N135, D108, K142, K166, E170, A173, Q200, C207, N240, and K260 in SEQ ID NO: 3; a mutation at one or more positions corresponding to I21, N44, N47, S53, K57, I104, D108, N135, S140, K142, K166, I68, E170, A173, Q200, C207, N240, and K260 in SEQ ID NO: 3; a mutation at one or more positions corresponding to I21, N44, N47, S53, K57, I68, I104, D108, V116, N135, S140, K142, K166, E170, A173, Q200, C207, N240, and K260 in SEQ ID NO: 3; a mutation at one or more positions corresponding to I21, N44, N47, S53, K57, A68, K104, D108, V16, N135, S140, K142, K166, E170, A173, Q200, M205, C207, N240, and K260 in SEQ ID NO: 3; a mutation at one or more positions corresponding to Q15, I121, N44, N47, S53, K57, N135, 668, 104, D108, E109, V116, S140, K142, K166, E170, A173, Q200, M205, N206, C207, N240, R243, and K260 in SEQ ID NO: 3; a mutation at one or more positions corresponding to Q15, I21, K26, N44, N47, S53, K57, 168, S83,1104, D108, E109, V116, K131, N135, S140, K142, T157, K166, E170, A173, M195, Q200, M205, N206, C207, N240, R243, and K260 in SEQ ID NO: 3; or a mutation at position K26 in SEQ ID NO: 3.

Exemplary CjCgtB variants of SEQ ID NO: 3 as described herein are outlined below in Table 3.

TABLE 3

| Design Number | Mutations within SEQ ID NO: 3 |
|---|---|
| 49 | K26N, S53A, K166D |
| 50 | S53A, K166E |
| 51 | K26N, S53A, C207K |
| 52 | S53A, C207K |
| 53 | K26N, S53A, K166D, C207K |
| 54 | S53A, K166D, C207K |
| 55 | K26N, S53A, K166D, A173N, C207K |
| 56 | K26N, S53A, K142D, K131Y, K166D, A173N, Q200K, C207K |
| 57 | K26N, S53A, K142D, K166D, E170K, A173N, Q200K, C207K |
| 58 | Q15K, I21L, S53A, K57E, K142D, K166D, E170K, A173N, Q200K, |
| | M205L, C207K |
| 59 | Q15K, K26N, S53A, K57E, K142D, K166D, I68L, N135Q, E170K, |
| | A173N, Q200K, C207K, N240E |
| 60 | K26N, N44K, S53A, K57E, I68L, N135Q, K142D, K166D, E170K, |
| | A173N, Q200K, C207K, N240E |
| 61 | Q15K, K26N, N44K, S53A, K57E, N135Q, K142D, K166D, I68L, |
| | E170K, A173N, Q200K, C207K, N240E, K260I |
| 62 | Q15K, I21L, K26N, N44K, S53A, K57E, I68L, N135Q, K142D, |
| | K166D, E170K, A173N, Q200K, C207K, N240E, K260I |
| 63 | Q15K, I21L, K26N, N44K, S53A, K57E, I68L, I104L, N135Q, |
| | K142D, K166D, E170K, A173N, Q200K, C207K, N240E, K260I |
| 64 | I21L, K26N, S53A, K57E, I68L, N44K, I104L, N135Q, D108N, |
| | K142D, K166D, E170K, A173N, Q200K, C207K, N240E, K260I |
| 65 | I21L, N44K, N47E, S53A, K57E, I104L, D108N, N135Q, S140N |
| | K142D, K166D, I68L, E170K, A173N, Q200K, C207K, N240E, |
| | K260I |
| 66 | I21L, N44K, N47E, S53A, K57E, I68L, I104L, D108N, V116I, |
| | N135Q, S140N, K142D, K166D, E170K, A173N, Q200K, C207K, |
| | N240E, K260I |
| 67 | I21L, N44K, N47E, S53A, K57E, 168L, I104L, D108N, V116I, |
| | N135Q, S140N, K142D, K166D, E170K, A173N, Q200K, M205L, |
| | C207K, N240E, K260I |
| 68 | Q15K, I21L, N44K, N47E, S53A, K57E, N135Q, I68L, I104L, |
| | D108N, E109T, V116I, S140N, K142D K166D, E170K, A173N, |
| | Q200K, M205L, N206Q, C207K, N240E, R243K, K260I |
| 69 | Q15K, I21L, K26N, N44K, N47E, S53A, K57E, I68L, S83A, I104L, |
| | D108N, E109T, V116I, K131Y, N135Q, S140N, K142D, T157N, |
| | K166D, E170K, A173N, M195I, Q200K, M205L, N206Q, C207K, |
| | N240E, R243K, K260I |
| 70 | K26N |

### V. Recombinant Nucleic Acids

In a related aspect, provided herein are nucleic acids encoding CjCgtA and/or CjCgtB variants as described herein. The nucleic acids can be generated from a nucleic acid template encoding the wild-type CjCgtA and/or CjCgtB, using a number of recombinant DNA techniques that are known to those of skill in the art.

In some examples, described herein is an isolated CjCgtA and/or CjCgtB nucleic acid having at least about 80%, e.g., at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94% at least about 95% at least about 96%, at least about 97%, at least about 98%, or at least about 99%, sequence identity to any one of the nucleic acid sequences set forth in SEQ ID NO: 1, 2, 3, or 23. In some examples, the isolated nucleic acid comprises the polynucleotide sequence set forth in SEQ ID NO: 1. In some examples, the isolated nucleic acid comprises the polynucleotide sequence set forth in SEQ ID NO: 2. In some examples, the isolated nucleic acid comprises the polynucleotide sequence set forth in SEQ ID NO: 3. In some examples, the isolated nucleic acid comprises the polynucleotide sequence set forth in SEQ ID NO: 23.

Using a CjCgtA and/or CjCgtB nucleic acid of the disclosure, a variety of expression constructs and vectors can be made. Generally, expression vectors include transcriptional and translational regulatory nucleic acid regions operably linked to the nucleic acid encoding the mutant glycosyltransferase. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. In addition, the vector may contain a Positive Retroregulatory Element (PRE) to enhance the half-life of the transcribed mRNA (see, Gelfand et al. U.S. Pat. No. 4,666,848). The transcriptional and translational regulatory nucleic acid regions will generally be appropriate to the host cell used to express the glycosyltransferase. Numerous types of appropriate expression vectors and suitable regulatory sequences are known in the art for a variety of host cells. In general, the transcriptional and translational regulatory sequences may include, e.g., promoter sequences, ribosomal binding sites, transcriptional start and stop sequences, translational start and stop sequences, and enhancer or activator sequences. Typically, the regulatory sequences will include a promoter and/or transcriptional start and stop sequences. Vectors also typically include a polylinker region containing several restriction sites for insertion of foreign DNA. As described above, heterologous sequences (e.g., a fusion tag such as a His tag) can be used to facilitate purification and, if desired, removed after purification. The construction of suitable vectors containing DNA encoding replication sequences, regulatory sequences, phenotypic selection genes, and the mutant glycosyltransferase of interest are prepared using standard recombinant DNA procedures. Isolated plasmids, viral vectors, and DNA fragments are cleaved, tailored, and ligated together in a specific order to generate the desired vectors, as is well-known in the art (see, e.g., Sambrook et al., Molecular Cloning: A Laboratory Manual (Cold Spring Harbor Laboratory Press, New York, NY, 2nd ed. 1989)).

Accordingly, some examples of the disclosure provide an expression cassette comprising a CjCgtA and/or CjCgtB nucleic acid as described herein operably linked to a promoter. Provided also herein is a vector comprising CjCgtA and/or CjCgtB nucleic acid as described herein. In some examples, the CjCgtA and/or CjCgtB nucleic acid in the expression cassette or vector comprises the polynucleotide sequence set forth in SEQ ID NO: 1, SEQ ID NO: 2, SEQ ID: 3, or SEQ ID NO: 23.

### VI. Host Cells

In certain examples, the expression vector contains a selectable marker gene to allow the selection of transformed host cells. Selection genes are well known in the art and will vary with the host cell used. Suitable selection genes can include, for example, genes coding for ampicillin and/or tetracycline resistance, which enables cells transformed with these vectors to grow in the presence of these antibiotics.

In one aspect, a nucleic acid encoding a glycosyltransferase as described herein is introduced into a cell, either alone or in combination with a vector. By “introduced into” or grammatical equivalents herein is meant that the nucleic acids enter the cells in a manner suitable for subsequent integration, amplification, and/or expression of the nucleic acid. The method of introduction is largely dictated by the targeted cell type. Exemplary methods include CaPO₄ precipitation, liposome fusion, LIPOFECTIN®, electroporation, heat shock, viral infection, and the like.

In some examples, prokaryotes are used as host cells for the initial cloning steps as described herein. Other host cells include, but are not limited to, eukaryotic (e.g., mammalian, plant and insect cells), or prokaryotic (bacterial) cells. Exemplary host cells include, but are not limited to, Escherichia coli, Saccharomyces cerevisiae, Pichia pastoris, Sf9 insect cells, and CHO cells. They are particularly useful for rapid production of large amounts of DNA, for production of single-stranded DNA templates used for site-directed mutagenesis, for screening many mutants simultaneously, and for DNA sequencing of the mutants generated. Suitable prokaryotic host cells include E. coli K12 strain 94 (ATCC No. 31,446), E. coli strain W3110 (ATCC No. 27,325), E. coli K12 strain DG116 (ATCC No. 53,606), E. coli X1776 (ATCC No. 31,537), and E. coli B; and other strains of E. coli, such as HB101, JM101, NM522, NM538, and NM539. Many other species and genera of prokaryotes including bacilli such as Bacillus subtilis, other enterobacteriaceae such as Salmonella typhimurium or Serratia marcesans, and various Pseudomonas species can all be used as hosts. Prokaryotic host cells or other host cells with rigid cell walls are typically transformed using the calcium chloride method as described in Sambrook et al., supra. Alternatively, electroporation can be used for transformation of these cells. Prokaryote transformation techniques are set forth in, for example Dower, in Genetic Engineering, Principles and Methods 12:275-296 (Plenum Publishing Corp., 1990); Hanahan et al., Meth. Enzymol., 204:63, 1991. Plasmids typically used for transformation of E. coli include pBR322, pUCI8, pUCI9, pUCIl8, pUC119, and Bluescript M13, all of which are described in sections 1.12-1.20 of Sambrook et al., supra. However, many other suitable vectors are available as well.

Accordingly, some examples described herein provide a host cell comprising a CjCgtA and/or CjCgtB nucleic acid, expression cassette, or vector, as described herein. In some examples, the CjCgtA and/or CjCgtB nucleic acid, expression cassette, or vector in the host cell encodes a polypeptide comprising the amino acid sequence set forth in SEQ ID NO: 1, SEQ ID NO: 2, SEQ ID NO: 3, or SEQ ID NO: 23.

In some examples, the CjCgtA and/or CjCgtB variants described herein are produced by culturing a host cell transformed with an expression vector containing a nucleic acid encoding the glycosyltransferase, under the appropriate conditions to induce or cause expression of the glycosyltransferase. Methods of culturing transformed host cells under conditions suitable for protein expression are well-known in the art (see, e.g., Sambrook et al., supra).

Following expression, a CjCgtA and/or CjCgtB variant can be harvested and isolated. In some examples, the present disclosure provides a cell including a recombinant nucleic acid as described herein. The cells can be prokaryotic or eukaryotic. The cells can be mammalian, plant, bacteria, or insect cells.

### VII. Methods of Making Oligosaccharides

The glycosyltransferases described herein can be used to prepare oligosaccharides, specifically to add N-acetylneuraminic acid (Neu5Ac), other sialic acids, and analogs thereof, to a monosaccharide, an oligosaccharide, a glycolipid, a glycopeptide, or a glycoprotein. Described herein is a multistep one-pot multienzyme (MSOPME) strategy has been successfully developed for enzymatic synthesis of glycosphingosines from precursor materials, e.g., from lactosylsphingosine. Optionally, the methods are performed without the purification of intermediate glycosphingosines. The methods described herein, in combination with the glycosyltransferase engineering strategies and resulting enzyme variants as described above, provide quick access to GM1 gangliosides containing different sialic acid forms. For example, the methods and enzymes described herein can be applied to synthesizing a variety of glycosphingolipids, glycoconjugates, and glycans.

In some examples, a method for preparing a glycosylated molecule is provided here. The method includes the steps of forming a reaction mixture comprising a glycosylation donor comprising a sugar component, a glycosylation acceptor comprising a sphingosine moiety, and a glycosyltransferase, wherein the glycosyltransferase is a CjCgtA variant or a CjCgtB variant as described herein, and maintaining the reaction mixture under conditions suitable for forming a glycosylated molecule. In the maintaining step, the conditions are sufficient to transfer the sugar moiety from the glycosylation donor to the glycosylation acceptor, thereby forming the glycosylated molecule.

The glycosylation acceptor can be any suitable oligosaccharide, glycolipid, glycopeptide, or glycoprotein. When the acceptor sugar is an oligosaccharide, any suitable oligosaccharide can be used. For example, the acceptor sugar can be a Neu5Gc-containing GM3 sphingosine (e.g., Neu5Gc-GM3βSph).

The glycosylation donor includes a nucleotide and sugar. Any nucleotide can be used, include, but are not limited to, adenine, guanine, cytosine, uracil and thymine nucleotides with one, two or three phosphate groups. In some examples, the nucleotide can be cytidine monophosphate (CMP).

Any glycosyltrasferase as described herein can be used in the present methods. In some examples, the glycosyltransferase is a CjCgtA variant. In other examples, the glycosyltransferase is a CjCgtB variant. Optionally, the glycosyltransferase can include a polypeptide sequence according to SEQ ID NO: 1. Optionally, the glycosyltransferase can include a polypeptide sequence according to SEQ ID NO: 2. Optionally, the glycosyltransferase can include a polypeptide sequence according to SEQ ID NO: 3. Optionally, the glycosyltransferase can include a polypeptide sequence according to SEQ ID NO: 23.

The glycosyltrasferases can be, for example, purified prior to addition to the reaction mixture or secreted by a cell present in the reaction mixture. In some cases, the glycosyltransferases can catalyze the reaction within a cell expressing the glycosyltransferase.

In some cases, a detergent can be added the reaction mixture. The addition of a detergent can improve the glycosylation efficiency of glycosphingosines by CjCgtA and CjCgtB. Optionally, the detergent is an anionic detergent (e.g., sodium cholate). Optionally, the detergent is a non-ionic detergent (e.g., Triton X-100; Dow Chemical Company, Midland, MI). The detergent can be used at any suitable concentration, which can be readily determined by one of skill in the art. For example, one or more detergents can be included in the reaction mixtures at concentrations ranging from about 0.1 mM to about 30 mM (e.g., from about 1 mM to about 20 mM, from about 5 mM to about 15 mM, or from about 6 mM to about 12 mM). For example, one or more detergents can be included in a reaction mixture at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, or 30 mM.

Reaction mixtures can also contain additional reagents for use in glycosylation techniques. For example, in certain examples, the reaction mixtures can contain buffers (e.g., 2-(N-morpholino)ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, and sodium borate), cosolvents (e.g., dimethylsulfoxide, dimethylformamide, ethanol, methanol, tetrahydrofuran, acetone, and acetic acid), salts (e.g., NaCl, KCl, CaCl₂), and salts of Mn²⁺ and Mg²⁺), chelators (e.g., ethylene glycol-bis(2-aminoethylether)-N,N,N,N-tetraacetic acid (EGTA), 2-({2-[Bis(carboxymethyl)amino]ethyl}(carboxymethyl)amino)acetic acid (EDTA), and 1,2-bis(o-aminophenoxy)ethane-N,N,N,N-tetraacetic acid (BAPTA)), reducing agents (e.g., dithiothreitol (DTT), P-mercaptoethanol (BME), and tris(2-carboxyethyl)phosphine (TCEP)), and/or labels (e.g., fluorophores, radiolabels, and spin labels). Buffers, cosolvents, salts, chelators, reducing agents, and/or labels can be used at any suitable concentration, which can be readily determined by one of skill in the art. In general, buffers, cosolvents, salts, chelators, reducing agents, and labels are included in reaction mixtures at concentrations ranging from about 1 M to about 1 M. For example, a buffer, a cosolvent, a salt, a chelator, a reducing agent, or a label can be included in a reaction mixture at a concentration of about 1 μM, or about 10 μM, or about 100 M, or about 1 mM, or about 10 mM, or about 25 mM, or about 50 mM, or about 100 mM, or about 250 mM, or about 500 mM, or about 1 M.

Reactions are conducted under conditions sufficient to transfer the sugar moiety from a glycosylation donor to a glycosylation acceptor. The reactions can be conducted at any suitable temperature. In general, the reactions are conducted at a temperature of from about 4° C. to about 40° C. The reactions can be conducted, for example, at about 25° C. or about 37° C. The reactions can be conducted at any suitable pH. In general, the reactions are conducted at a pH of from about 4.5 to about 10. The reactions can be conducted, for example, at a pH of from about 5 to about 9. The reactions can be conducted for any suitable length of time. In general, the reaction mixtures are incubated under suitable conditions for anywhere between about 1 minute and several hours. The reactions can be conducted, for example, for about 1 minute, or about 5 minutes, or about 10 minutes, or about 30 minutes, or about 1 hour, or about 2 hours, or about 4 hours, or about 8 hours, or about 12 hours, or about 24 hours, or about 48 hours, or about 72 hours. Other reaction conditions may be employed in the methods described herein, depending on the identity of a particular glycosyltransferase, glycosylation donor, or glycosylation acceptor.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1: Glycosyltransferase Engineering and Characterization

### Protein Expression and Purification

Recombinant enzymes were expressed and purified. Briefly, E. coli BL21 (DE3) cells harboring the recombinant plasmid containing the target gene were cultured in Luria-Bertani (LB) broth (10 g L⁻¹ tryptone, 5 g L⁻¹ yeast extract, and 10 g L⁻¹ NaCl) containing ampicillin (0.1 mg mL⁻¹) with rapid shaking (220 rpm) at 37° C. for overnight. Then, the overnight culture (5 mL) was transferred into 1 L of LB broth containing ampicillin (0.1 mg mL⁻¹) and incubated at 37° C. When the OD_{600 nm} of the cell culture reached 0.6-0.8, isopropyl-1-thio-o-D-galactopyranoside (IPTG, 0.1 mM) was added to induce the expression of the recombinant enzyme. The culture was then incubated at 20° C. with shaking (220 rpm) for 20 hours. Cells were collected by centrifugation at 4392×g for 30 min at 4° C. The cell pellet was re-suspended in lysis buffer (100 mM Tris-HCl buffer, pH 7.5, containing 0.1% Triton X-100) and the cells were lysed using a homogenizer (EmulsiFlex-C3; Avestin, Ottawa, Canada). Cell lysate was obtained by centrifugation at 9016×g for 1 hour at 4° C. The supernatant was filtered using a 0.45 μm syringe filter and loaded to a nickel-nitrilotriacetic acid (Ni²⁺-NTA) affinity column pre-equilibrated with a binding buffer (50 mM Tris-HCl buffer, pH 7.5, 5 mM imidazole, 0.5 M NaCl). The column was washed with 10 column volumes of a binding buffer and 10 column volumes of a washing buffer (50 mM Tris-HCl buffer, pH 7.5, 10 mM imidazole, 0.5 M NaCl) and eluted using 10 column volumes of an elution buffer (50 mM Tris-HCl buffer, pH 7.5, 200 mM imidazole, 0.5 M NaCl). Fractions containing the target protein were combined and dialyzed against a dialysis buffer (20 mM Tris-HCl buffer, pH 7.5, 10% glycerol). The samples were then stored at −20° C.

### Plasmid Construction for MBP-Δ15CjCgtA-His₆

To construct the plasmid for expressing MBP-Δ15CjCgtA-His₆, the Δ15CjCgtA-His₆ gene in a pET22b(+) vector plasmid was subcloned into pMAL-c2X vector. The primers used were: Forward, 5′-GACCGAATTC GTGCTGGACAACGAGCAC-3′ (EcoRI restriction site is underlined; SEQ ID NO: 8); Reverse, 5′-CAGCAAGCTTTCAGTGGTGGTGGTGGTG-3′ (HindIII restriction site is underlined; SEQ ID NO: 9). The polymerase chain reaction (PCR) for amplifying the target gene was performed in a 50 μL reaction mixture containing the plasmid DNA (10 ng), forward and reverse primer (0.2 μM each), 1× Phusion HF buffer, dNTP mixture (0.2 mM each), and 1 U (0.5 μL) of Phusion® High-Fidelity DNA Polymerase. The reaction mixture was subjected to 30 cycles of amplification at an annealing temperature of 55° C. The resulting PCR product was purified and double digested with EcoRI and HindIII restriction enzyme. The digested and purified PCR product was inserted by ligating with the pMAL-c2X vector predigested with the same restriction enzymes and transformed into E. coli DH5α Z-competent cells. Selected clones were grown for plasmid minipreps and the gene sequence was confirmed by customer sequencing by Genewiz (South Plainfield, NJ).

### Plasmid Construction for MBP-CjCgtBΔ30-His₆

To construct the plasmid for expressing MBP-CjCgtBΔ30-His₆, the CjCgtBΔ30-His₆ gene in a pET22b(+) vector plasmid was subcloned into pMAL-c2X vector. The primers used were: Forward, 5′-GACCGAATTCTTCAAAATTTCTATCATCCTGCCG 3′ (EcoRI restriction site is underlined; SEQ ID NO: 10); Reverse, 5′-CAGCAAGCTTTTAGTGGTGGTGATGATGATGCTTAATTTTGTAGATCTGAATATA C-3′ (HindIII restriction site is underlined; SEQ ID NO: 11). The PCR for amplifying the target gene was performed similarly to that described above except that 52° C. was used as the annealing temperature. The subcloning process and gene sequence confirmation were the same as described above.

MBP-Δ15CjCgtA-His₆ and MBP-CjCgtBΔ30-His₆ Expression and Purification

Escherichia coli BL21(DE3) cells were transformed with the desired plasmid and grown on an LB agar plate containing ampicillin (0.1 mg mL⁻¹). A single colony was inoculated in LB broth supplemented with 0.1 mg mL⁻¹ ampicillin. The protein expression and purification procedures were similar to that described above for other enzymes. The enzymes were dialyzed against a buffer containing Tris-HCl (50 mM, pH 7.5) and NaCl (250 mM). The dialyzed samples were either lyophilized or added with 10% of glycerol, and then stored at −20° C.

### Enzyme Activity Assays

The enzymatic assays were carried out in duplicate at 37° C. for 10 minutes in a reaction mixture (10 μL) containing the donor substrate (1.5 mM, UDP-GalNAc for MBP-Δ15CjCgtA-His₆ and UDP-Gal for MBP-CjCgtBΔ30-His₆), an acceptor substrate (1 mM, GM3βNHCbz for MBP-Δ15CjCgtA-His₆ and GM2βNHCbz for MBP-CjCgtBΔ30-His₆), Tris-HCl buffer (100 mM, pH 7.5), a metal cation (10 mM, MgCl₂ for MBP-Δ15CjCgtA-His₆ and MnCl₂ for MBP-CjCgtBΔ30-His₆), and the enzyme (0.32 μM for MBP-Δ15CjCgtA-His₆ and 4 μM for MBP-CjCgtBΔ30-His₆). The reactions were stopped by adding 10 μL of ice-cold methanol followed by incubation of the mixture on ice for 20 minutes and centrifugation at 16200 g for 5 minutes. The supernatant (about 20 μL) was transferred into another tube containing ddH₂O (40 μL) and the resulting mixture was analyzed by liquid chromatography-mass spectrometry (LC-MS) (SHIMADZU LCMS-2020 system with electrospray ionization) for confirming the product and ultra-high-performance liquid chromatography (UHPLC) (monitored at 215 nm on an Agilent Infinity 1290 II HPLC system equipped with 1260 Infinity II Diode Array Detector WR) for reaction yield determination. The column used for the UHPLC analysis was Dionex™ CarboPac™ PA-100 (1.8 μm particle, 4×250 mm, Thermo Scientific, CA) for both glycosyltransferases. A gradient flow (100% water to 70% water/30% 1 M NaCl in 16 min) was used for analyzing the reactions catalyzed by MBP-Δ15CjCgtA-His₆ and a different gradient flow (100% water to 75% water/15% 1 M NaCl in 16 min) was used for analyzing MBP-CjCgtBΔ30-His₆-catalyzed reactions. The flow rate was 0.75 mL min⁻¹.

### Results and Discussion

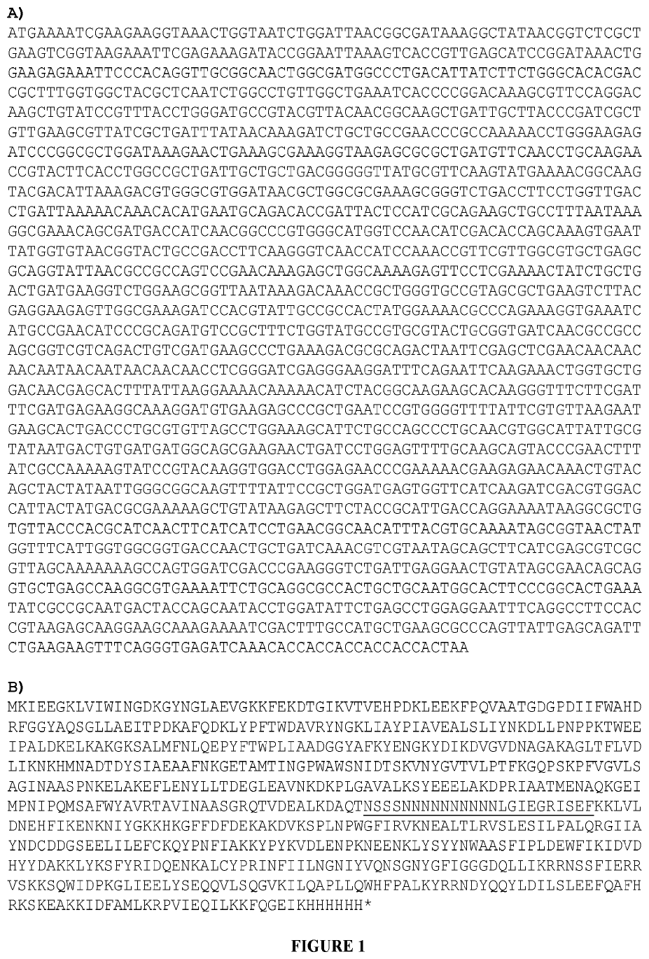

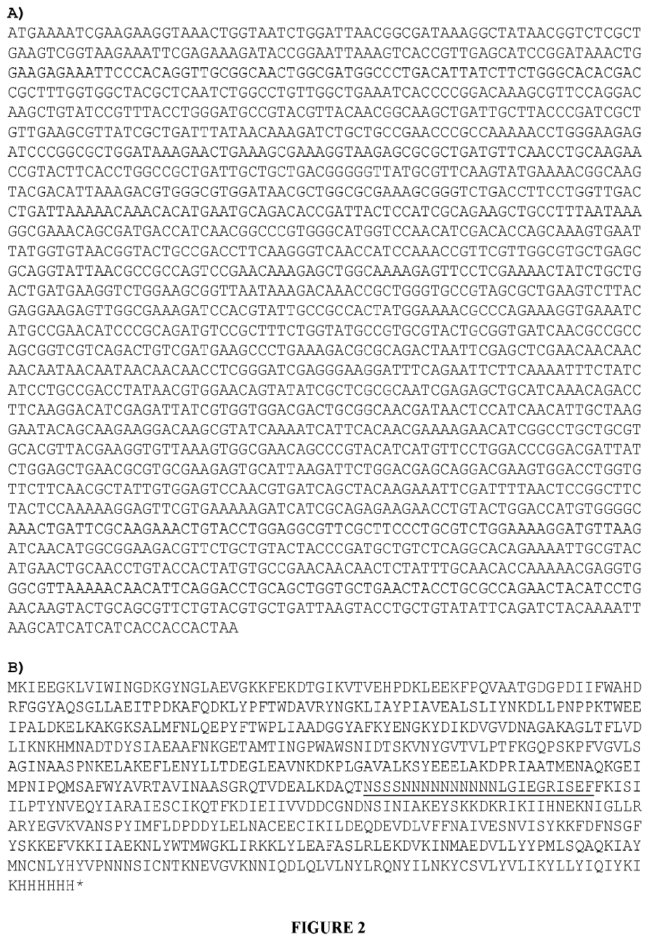

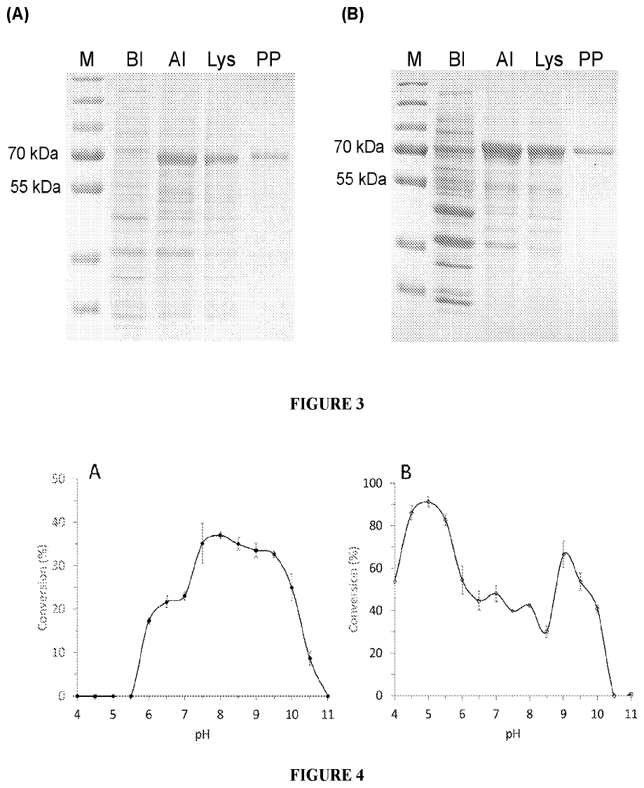

Campylobacter jejuni β1-4GalNAcT (CjCgtA) and β1-3-galactosyltransferase (CjCgtB) were cloned and expressed in Escherichia coli (E. coli) as N-terminal or C-terminal truncated, and C-terminal hexahistidine-tagged recombinant proteins. With an expression level of 40 mg purified protein per liter culture, Δ15CjCgtA-His₆ was not stable for storage at 4° C. and was easily precipitated during dialysis. Cell lysate instead of purified enzyme was used previously for enzymatic synthesis. On the other hand, CjCgtBΔ30-His₆ with an expression level of 20 mg purified protein per liter culture was more stable but its expression level was not as high. To improve their soluble expression levels and stability, a maltose-binding protein (MBP) was fused to the N-terminus of Δ15CjCgtA-His₆ and CjCgtBΔ30-His ₆. The resulting recombinant MBP-Δ15CjCgtA-His₆ (FIG. 1, Panels A and B; see also SEQ ID NO: 4 and SEQ ID NO: 5, respectively) and MBP-CjCgtBΔ30-His₆ (FIG. 2, Panels A and B; see also SEQ ID NO: 6 and SEQ ID NO: 7, respectively) with expression levels of 85 mg L⁻¹ culture and 110 mg L⁻¹ culture, respectively (FIG. 3, Panels A and B), were stable throughout the nickel-nitrilotriacetate (Ni²⁺-NTA) column purification and dialysis processes. Both could be lyophilized without losing enzymatic activity.

### Example 2: pH Profile

Enzymatic assays were performed in a buffer (100 mM) with a pH in the range of 3.0-10.0. Buffers used were: citric acid-sodium citrate, pH 4.0-5.5; PBS, pH 6.0-7.0; Tris-HCl, pH 7.5-8.5; and glycine-NaOH, pH 9.0-11.0. The MBP-Δ15CjCgtA-His₆ reactions were performed in the presence of MgCl₂ (10 mM) and the MBP-Δ15CjCgtA-His₆ reactions were performed in the presence of MnCl₂ (10 mM).

MBP-Δ15CjCgtA-His₆ was shown to be active in a broad pH range of pH 6.0-10.5 and optimal activity was found in pH 7.5-9.5 (FIG. 4, Panel A, electrospray ionization (ESI)). MBP-CjCgtBΔ30-His₆ was also active in a broad pH range (pH 4.5-10.0) with the optimal activity in pH 4.5-5.5 (FIG. 4, Panel B, ESI).

### Example 3: Effects of Divalent Metal Cations, Ethylenediaminetetraacetic Acid (EDTA), and Dithiothreitol (DTT)

The effect of various metal ions, the chelating reagent EDTA, and the reducing reagent DTT on the enzyme activity of MBP-Δ15CjCgtA-His₆ and MBP-CjCgtBΔ30-His₆ were examined at pH 7.5 in a Tris-HCl buffer (100 mM). Reactions without metal ions were used as controls.

Both MBP-Δ15CjCgtA-His₆ and MBP-CjCgtBΔ30-His₆ required a divalent metal cation for activity (FIG. 5, Panels A and B ESI). Mn²⁺ was a preferred cation for both. Mg²⁺ was equally effective for MBP-Δ15CjCgtA-His₆ but was less effective for MBP-CjCgtBΔ30-His₆. Ca²⁺ was suitable for MBP-Δ15CjCgtA-His₆ but not for MBP-CjCgtBΔ30-His₆. The addition of dithiothreitol (DTT, 10 mM) deactivated MBP-Δ15CjCgtA-His₆ but improved the activity of MBP-CjCgtBΔ30-His₆ (FIG. 5, Panels A and B, ESI).

### Example 4: Thermostability Studies

Thermostability studies of MBP-Δ15CjCgtA-His₆ (in the presence of 10 mM MgCl₂) and MBP-CjCgtBΔ30-His₆ (in the presence of 10 mM MnCl₂) were performed by incubating the enzyme in a Tris-HCl buffer (100 mM, pH 7.5) at different temperatures for different durations (1 hour, 3 hours, 15 hours, and 24 hours) in the reaction buffer. The substrates were then added and the reaction mixtures were incubated at 37° C. for 10 minutes followed by reaction quenching and sample analyses.

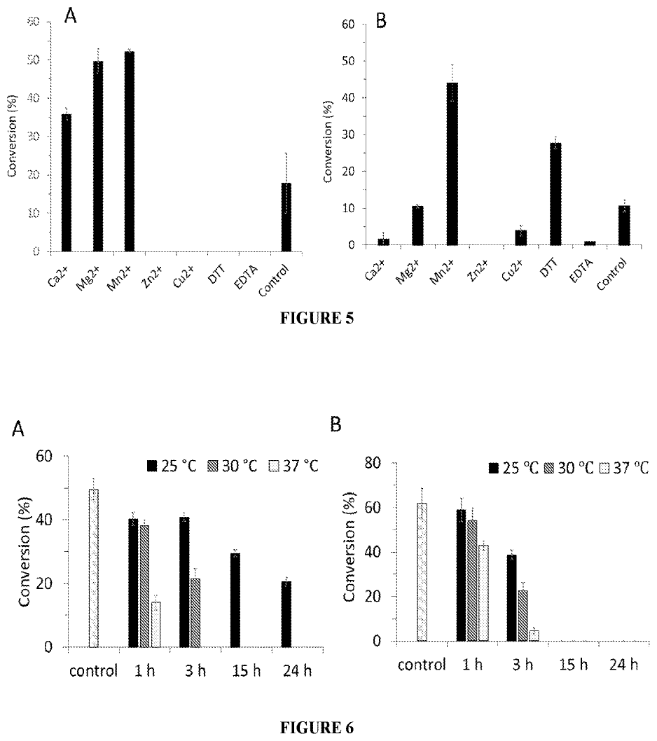

Thermostability assays (FIG. 6, Panels A and B, ESI) showed that purified and dialyzed MBP-Δ15CjCgtA-His₆ and MBP-CjCgtBΔ30-His₆ samples lost most catalytic activity after incubating at 37° C. for 3 hours while about 50% activity was retained after incubation at 30° C. for 3 hours and 30° C. was chosen as a more suitable reaction temperature for enzymatic synthetic purpose.

### Example 5: Effects of Detergents on MBP-Δ15CjCgtA-His₆-Catalyzed OPME Formation of GM2βSph

Assays were carried out at 30° C. for 12 hours in a total volume of 10 μL in Tris-HCl buffer (100 mM, pH 7.5) containing a GM3βSph (10 mM), GalNAc (15 mM), ATP (15 mM), UTP (15 mM), MgCl₂ (20 mM), BLNahK (2 μg), PmGlmU (2 μg), MBP-Δ15CjCgtA-His₆ (3 μg), PmPpA (1 μg), and various concentrations (0, 1, 2, 4, 5, 8, 10, 15, 18 mM) of sodium cholate or Triton X-100 (1, 5, 10, 15 mM). Reactions were quenched by addition of 10 μL of pre-chilled ethanol and the mixtures were incubated at 0° C. for 30 min, centrifuged, and the supernatant were analyzed by high resolution mass spectrometry (HRMS).

Glycosphingosines are much weaker acceptor substrates than the corresponding glycans for both CjCgtA and CjCgtB. This property made it prohibitive for synthesizing GM2βSph and GM1βSph in large-scales using the OPME strategy. However, GM2βSph (120 mg) and GM1βSph (57 mg) were synthesized in preparative scales and high yields were achieved with the use of a large amount of glycosyltransferases and relatively long reaction times. An anionic detergent, sodium cholate, and a non-ionic detergent Triton X-100 were shown to improve the activity of some enzymes which use glycosphingolipids as substrates. The effects of these detergents in influencing the activities of both MBP-Δ15CjCgtA-His₆ and MBP-CjCgtBΔ30-His₆ in using glycosphingosine acceptor substrates and the reactions were analyzed by high-resolution mass spectrometry (HRMS). It was found that the addition of sodium cholate in a concentration of 8-10 mM greatly enhanced the reaction yields of both enzymes. The non-ionic detergent Triton X-100 (10 mM) also improved the enzyme activities in using the glycosphingosine acceptor substrates although the effect was slightly less compared to that from sodium cholate at the same molar concentration.

### Example 6: MSOPME gram-scale synthesis of Neu5Ac-GM1βSph from LacβSph

### Materials and Methods

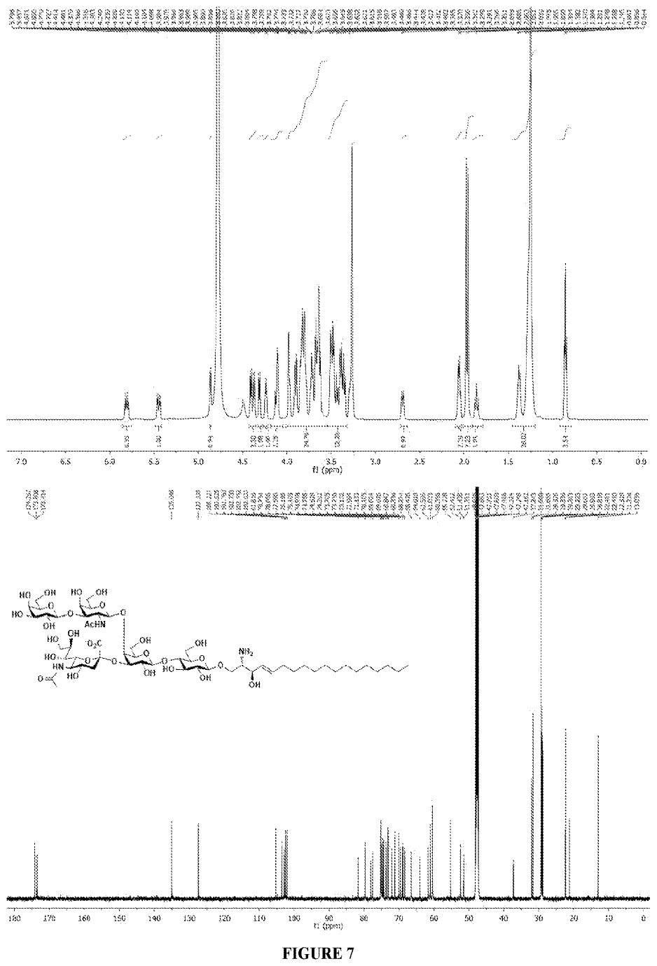

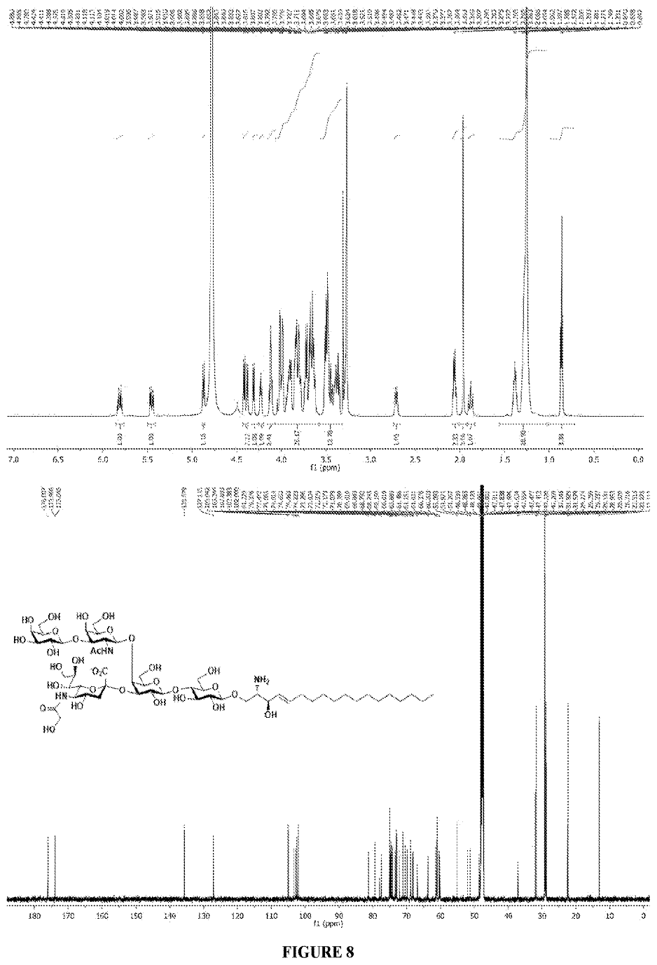

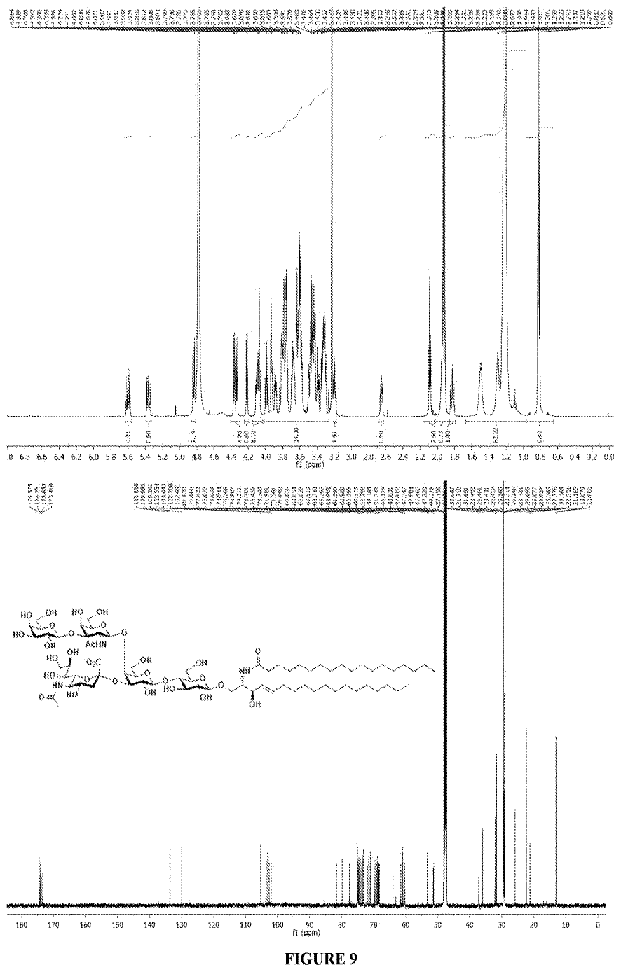

LacβSph (1.0 g, 1.6 mmol), Neu5Ac (0.64 g, 2.1 mmol), and CTP (1.45 g, 2.6 mmol) were incubated at 30° C. in a Tris-HCl buffer (150 mL, 100 mM, pH 8.5) containing MgCl₂ (20 mM), NmCSS (12 mg), and PmST3 (33 mg). The reaction was incubated in an incubator shaker at 30° C. with agitation at 100 rpm. The product formation was monitored by mass spectrometry. After 15 hours, an additional amount of PmST3 (10 mg) was added. After 2 days, the TLC and FIRMS indicated that the LacβSph was consumed almost completely. GalNAc (462 mg, 2.1 mmol), ATP (1.33 g, 2.4 mmol), and UTP (1.32 g, 2.4 mmol) were added, and the pH of the mixture was adjusted to 7.5 by adding NaOH (4 M). BLNahK (8 mg), PmGlmU (10 mg), MBP-Δ15CjCgtA-His₆ (16 mg), PmPpA (8 mg), and 1.5 mL of sodium cholate (1 M in water) were then added and the reaction mixture was incubated at 30° C. with agitation at 100 rpm. The product formation was monitored by TLC and FIRMS. When GM3βSph was completely consumed (30 hours), in the same reaction container without workup or purification, galactose (375 mg, 2.1 mmol), ATP (1.33 g, 2.4 mmol), and UTP (1.32 g, 2.4 mmol), SpGalK (10 mg), BLUSP (10 mg), MBP-CjCgtBΔ30-His₆ (12 mg), and PmPpA (8 mg) were added. The reaction mixture was incubated at 30° C. with agitation at 180 rpm for overnight. The product formation was monitored by FIRMS. After the reaction was completed (18 hours), the reaction mixture was incubated in a boiling water bath for 5 min and then centrifuged to remove precipitates. The supernatant was concentrated, and the residue was purified by passing through an ODS-SM column (50 μM, 120 A, Yamazen) using a CombiFlash® Rf 200i system. The fractions containing the product were collected and concentrated. The residue was further purified by silica gel column chromatography. A mixed solvent chloroform:methanol=5:2 (by volume) was used to remove sodium cholate and then chloroform:methanol:water=5:4:1 (by volume) was used as an eluant to produce pure Neu5Ac-containing GM1βSph (1.88 g, 90%) as a white powder. ¹H NMR (600 MHz, MeOD) δ 5.81 (dt, J=15.0, 7.2 Hz, 1H), 5.45 (dd, J=15.0, 7.2 Hz, 1H), 4.86 (d, J=9.0 Hz, 1H), 4.41 (d, J=7.8 Hz, 1H), 4.37 (d, J=7.8 Hz, 1H), 4.31 (d, J=7.8 Hz, 1H), 4.25-4.08 (m, 3H), 4.01-3.24 (m, 32H), 2.69 (dd, J=12.6, 5.4 Hz, 1H), 2.06 (q, J=7.2 Hz, 2H), 1.98 (s, 3H), 1.96 (s, 3H), 1.86 (t, J=12.0 Hz, 1H), 1.45-1.20 (m, 22H), 0.86 (t, J=7.2 Hz, 3H). ¹³C NMR (150 MHz, MeOD) δ 174.26, 173.81, 173.41, 135.05, 127.34, 105.22, 103.52, 102.73, 102.44, 102.03, 81.62, 79.73, 78.06, 77.60, 75.19, 75.11, 74.98, 74.76, 74.52, 74.29, 73.70, 73.22, 73.13, 71.99, 71.13, 70.10, 69.62, 69.08, 68.85, 68.34, 68.27, 66.48, 64.02, 61.60, 61.02, 60.40, 55.23, 52.41, 51.44, 51.35, 37.24, 31.98, 31.66, 29.38, 29.34, 29.26, 29.22, 29.05, 28.99, 28.82, 22.41, 22.32, 21.23, 13.04. HRMS (ESI-Orbitrap) m/z: [M−H]⁻ calculated for C₅₅H₉₆N₃O₃₀ 1278.6084, found 1278.6070. See FIG. 7 for ¹H and ¹³C NMR spectra of Neu5Ac-containing GM1 sphingosine (Neu5Ac-GM1βSph).

### Results and Discussion

With LacβSph (9) in hand and a good understanding of the optimal reaction conditions for both MBP-Δ15CjCgtA-His₆ and MBP-CjCgtBΔ30-His₆ including the benefit and the optimal concentration of a detergent, small-scale reactions were carried out to optimize the enzymatic synthesis of GM1 sphingosine (GM1βSph) from LacβSph (9) using three one-pot multienzyme (OPME) reaction systems including an OPME α2-3-sialylation (OPME1), an OPME β1-4-GalNAcylation (OPME2), and an OPME β1-3-galactosylation (OPME3) processes (Scheme 1). As GM1βSph is the target, it is not necessary to purify GM3βSph or GM2βSph intermediates after individual OPME reactions. Due to the non-overlapping acceptor substrate specificities of the glycosyltransferases involved (only the product of the previous OPME is the acceptor for the glycosyltransferase in the next OPME), it is not necessary to deactivating the enzymes after each OPME step for the synthesis of GM1βSph as described herein.

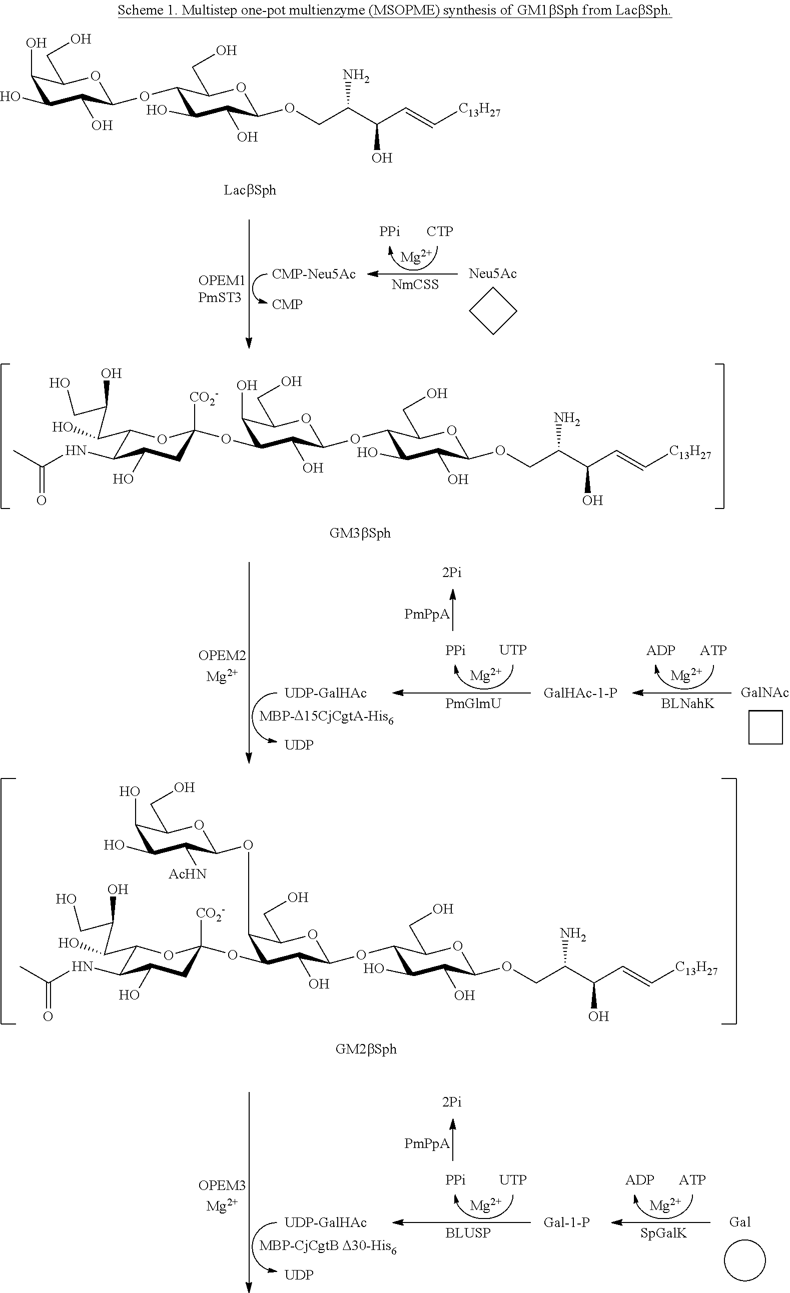

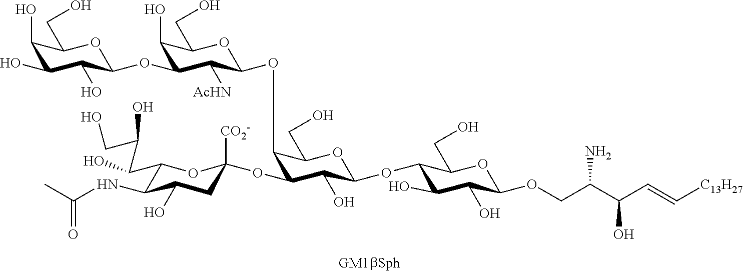

Starting from 100 mg of LacβSph (9) and Neu5Ac (1.3 eq.), GM3βSph was formed using OPME1 α2-3-sialylation reaction containing Neisseria meningitidis CMP-sialic acid synthetase (NmCSS) and Pasteurella multocida α2-3-sialyltransferase 3 (PmST3) (Scheme 1). The reaction was monitored by high-resolution mass spectrometry (HRMS) and went to completion in 20 hours at 30° C.

Without purification, the reaction mixture was used for OPME2 01-4-GalNAcylation reaction by adding GalNAc, ATP, UTP, sodium cholate (8 mM final concentration), and four enzymes including Bifidobacterium longum strain ATCC55813 N-acetylhexosamine-1-kinase (BLNahK), Pasteurella multocida N-acetylglucosamine uridylyltransferase (PmGlmU), Pasteurella multocida inorganic pyrophosphatase (PmPpA), and MBP-Δ15CjCgtA-His₆. The reaction mixture was incubated at 30° C. to generate GM2βSph. The presence of sodium cholate decreased the reaction time and the amount of MBP-Δ15CjCgtA-His₆ needed (compared to previous OPME synthesis of GM2βSph) to a level similar to GM2 glycan synthesis. The OPME2 reaction was completed in 20 hours at 30° C.

Without purification, the resulting reaction mixture was applied for OPME3 β1-3-galactosylation reaction in the third step by adding Gal, ATP, UTP, and four enzymes including Streptococcus pneumoniae TIGR4 galactokinase (SpGalK), Bifidobacterium longum UDP-sugar pyrophosphorylase (BLUSP), PmPpA, and MBP-CjCgtBΔ30-His₆. As sodium cholate was added in the second step, no additional detergent was needed in this step. 10 The formation of GM1βSph was completed in 16 hours at 30° C. Both the reaction time and the amount of MBP-CjCgtBΔ30-His₆ were decreased (compared to previous OPME synthesis of GM1βSph) due to the presence of sodium cholate.

The GM1βSph product purification was carried out by passing the reaction mixture through a C18 cartridge and eluting with a mixed solvent gradient of CH₃CN in water. It was found that GM1βSph could be separated efficiently from other components except for sodium cholate in the reaction mixture. The removal of sodium cholate from GM1βSph was achieved by silica gel column chromatography, in which sodium cholate was eluted out first using CHCl₃:MeOH=5:2 (by volume) and then GM1βSph was eluted using CHCl₃:MeOH:H₂O=5:4:1 (by volume).

Once the optimized synthetic procedures and purification processes were established, gram-scale synthesis of GM1βSph (1.88 g) from LacβSph (1.00 g) was carried out similarly (see ESI) and an excellent yield (90%) was achieved.

### Example 7: Multistep One-Pot Multienzyme (MSOPME) Synthesis of Neu5Gc-Containing GM1βSph (Neu5Gc-GM1βSph) from LacβSph

### Materials and Methods

A reaction mixture containing LacβSph (100 mg, 0.16 mM), ManNGc (57 mg, 0.24 mM), sodium pyruvate (176 mg, 1.60 mM), CTP (180 mg, 0.32 mM), MgCl₂ (20 mM), PmAldolase (3 mg), NmCSS (2 mg), and PmST3 (3 mg) in a Tris-HCl buffer (16 mL, 100 mM, pH 8.5) was incubated at 30° C. with agitation at 100 rpm. The product formation (Neu5Gc-GM3βSph) was monitored by mass spectrometry. After 24 hours, FIRMS indicated that the LacβSph was almost consumed. GalNAc (53 mg, 0.24 mmol), ATP (156 mg, 0.27 mmol), and UTP (150 g, 0.27 mmol) were then added and the pH of the reaction was adjusted to 7.5 by adding NaOH (4 M). After adding BLNahK (1.5 mg), PmGlmU (2 mg), MBP-Δ15CjCgtA-His₆ (4 mg), PmPpA (1 mg), and 0.16 mL of sodium cholate (1 M in water), the reaction mixture was incubated at 30° C. with agitation at 100 rpm. The product formation (Neu5Gc-GM2βSph) was monitored by FIRMS and Neu5Gc-GM3βSph was completely consumed after 12 hours. In the same reaction container without workup or purification, galactose (44 mg, 0.24 mmol), ATP (156 mg, 0.27 mmol), and UTP (150 g, 0.27 mmol), SpGalK (1.5 mg), BLUSP (1.5 mg), MBP-CjCgtBΔ30-His₆ (4 mg), and PmPpA (1 mg) were added. The reaction mixture was incubated at 30° C. for 12 hours with agitation at 180 rpm. The product formation was monitored by FIRMS. After the reaction is completed, the reaction mixture was incubated in a boiling water bath for 5 minutes and then centrifuged to remove precipitates. The supernatant was concentrated, and the residue obtained was purified by passing through an ODS-SM column (50 μM, 120 A, Yamazen) using a CombiFlash® Rf 200i system. The fractions containing the product were collected and concentrated. The residue was purified by silica gel column chromatography. A mixed solvent chloroform:methanol=5:2 (by volume) was used to remove sodium cholate and chloroform:methanol:water=5:4:1 (by volume) was used as an eluent to obtain pure Neu5Gc-GM10 Sph (193 mg, 91.4%) as a white powder. ¹H NMR (600 MHz, MeOD) δ 5.81 (dt, J=15.0, 7.2 Hz, 1H), 5.45 (dd, J=15.0, 7.2 Hz, 1H), 4.87 (d, J=8.7 Hz, 1H), 4.42 (d, J=7.8 Hz, 1H), 4.38 (d, J=7.8 Hz, 1H), 4.31 (d, J=7.8 Hz, 1H), 4.23 (t, J=6.0 Hz, 1H), 4.16-4.07 (m, 2H), 4.07-3.32 (m, 34H), 2.71 (dd, J=12.6, 5.4 Hz, 1H), 2.06 (q, J=7.2 Hz, 2H), 1.96 (s, 3H), 1.88 (t, J=12.0 Hz, 1H), 1.56-1.02 (m, 22H), 0.86 (t, J=7.2 Hz, 3H). ¹³C NMR (150 MHz, MeOD) δ 176.01, 173.87, 173.85, 135.68, 127.12, 105.04, 103.24, 102.69, 102.38, 102.00, 81.23, 79.38, 78.00, 77.47, 75.07, 74.81, 74.65, 74.46, 74.22, 73.30, 73.03, 72.98, 72.17, 71.08, 70.39, 69.81, 68.84, 68.79, 68.24, 68.15, 66.82, 63.69, 61.49, 61.15, 61.03, 60.38, 60.20, 55.09, 51.97, 51.27, 37.14, 31.93, 31.58, 29.27, 29.26, 29.24, 29.13, 28.95, 28.92, 28.72, 22.52, 22.27, 13.11. FIRMS (ESI-Orbitrap) m/z: [M−H]⁻ calculated for C₅₅H₉₆N₃O₃₁ 1294.6033, found 1294.6051. See FIG. 8 for ¹H and ¹³C NMR spectra of Neu5Gc-containing GM1 sphingosine (Neu5Gc-GM1βSph).

### Results and Discussion

The optimized procedures for synthesis and purification as described above were also applied for the production of Neu5Gc-GM1βSph.

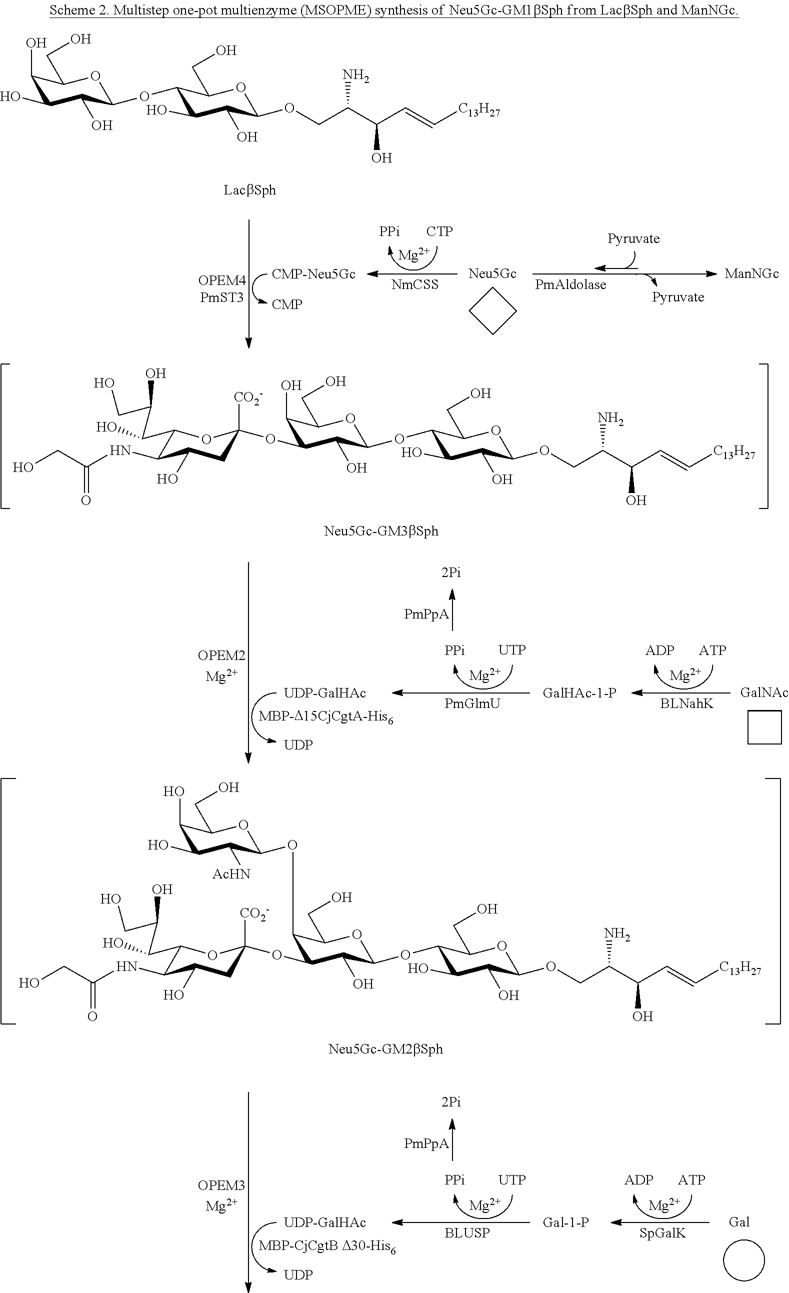

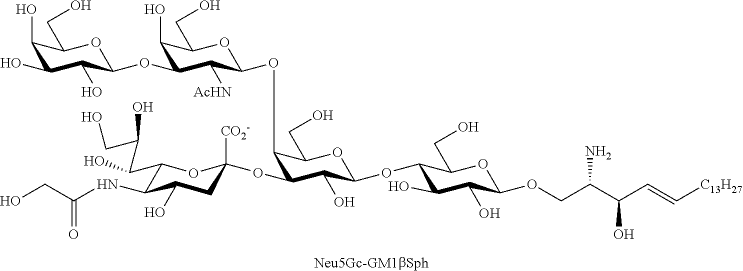

As shown in Scheme 2, Neu5Gc-containing GM3 sphingosine (Neu5Gc-GM3βSph) was readily synthesized from LacβSph as the acceptor substrate and N-glycolylmannosamine (ManNGc) as the Neu5Gc precursor using a three-enzyme OPME α2-3-sialylation system (OPME4) containing Pasteurella multocida sialic acid aldolase (PmNanA), NmCSS, and PmST3. Without purification, the reaction mixture was applied to the next step to produce Neu5Gc-GM2βSph via OPME2 with sodium cholate (10 mM). When the formation of Neu5Gc-GM2βSph was completed, the reaction mixture was directly applied into the next step without purification to produce Neu5Gc-GM1I Sph via OPME3. The desired Neu5Gc-GM1βSph was obtained in 91% yield after purification using a C18 cartridge followed by a silica gel column chromatography process.

### Example 8: One-Pot Preparative-Scale Enzymatic Synthesis of GM1βSph from GM3βSph

### Materials and Methods

GM3βSph (57 mg, 0.061 mmol), GalNAc (17.5 mg, 0.079 mmol), Gal (15 mg, 0.079 mmol), ATP (100 mg, 0.18 mmol), and UTP (100 mg, 0.18 mmol) were dissolved in water in a 50 mL centrifuge tube containing Tris-HCl buffer (100 mM, pH 7.5) and MgCl₂ (20 mM). The pH of the mixture was adjusted to 7.5 by adding NaOH (4 M). BLNahK (0.8 mg), PmGlmU (0.8 mg), SpGalK (0.8 mg), BLUSP (0.8 mg), MBP-Δ15CjCgtA-His₆ (1.2 mg), MBP-CjCgtBΔ30-His₆ (1.0 mg), PmPpA (0.5 mg), and 0.05 mL of sodium cholate (1 M in water) were then added and water was added to bring the final volume to 5 mL, resulting in a solution containing 12 mM GM3βSph. The reaction mixture was incubated at 30° C. in an incubator shaker with agitation at 180 rpm. The reaction was monitored by TLC assays and FIRMS analyses. After the reaction was completed (48 hours), the reaction mixture was incubated in a boiling water bath for 5 minutes, cooled down, and centrifuged. The supernatant was concentrated and purified similar to that described above to obtain pure GM1βSph (75 mg, 95%) as a white powder.

### Results and Discussion

The approach of synthesizing GM1βSph from GM3βSph in one-pot in a single step by adding all reagents and enzymes needed at the beginning was also analyzed. This approach worked well and the formation of GM1βSph from GM3βSph was completed in two days (Scheme 3). Employing the same C18 cartridge and silica gel column purification processes described above, pure GM1βSph product was obtained in 95% yield.

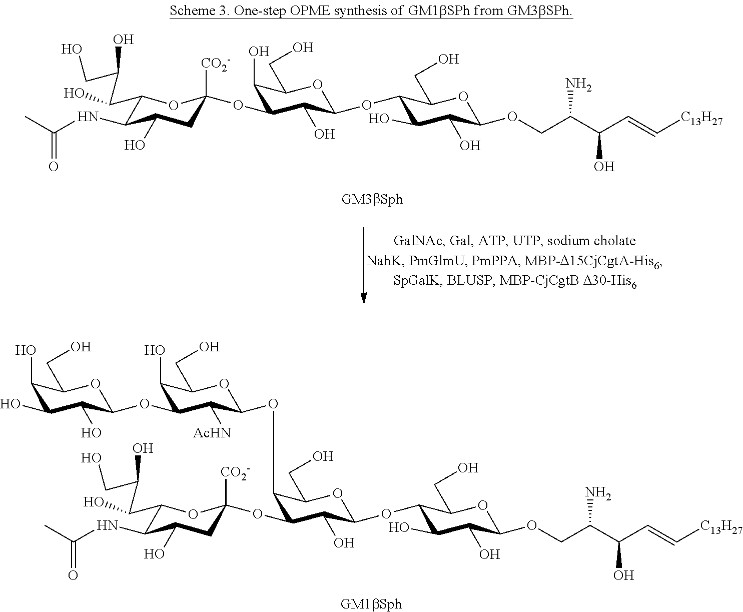

An attempt of producing GM1βSph directly from LacβSph in one-step by adding all reagents and enzymes at once produced GM1βSph with a moderate yield. It was found that the presence of sodium cholate slowed the sialylation process.

### Example 9: Synthesis of GM1 Gangliosides by Acylation of GM1 Sphingosines

### Synthetic Procedures

### GM1:

To a solution of GM1βSph (90 mg, 0.071 mmol) in sat. NaHCO₃-THF (4.5 mL, 2:1), stearoyl chloride (32 mg, 0.105 mmol, 1.5 eq) in 1.5 mL THF was added. The resulting mixture was stirred vigorously at room temperature for 2 hours. An additional 0.5 eq of stearoyl chloride was added and the mixture was stirred for another 2 hours and concentrated. The sample was loaded to a pre-conditioned (by washing the cartridge with three column volumes of MeOH and then three column volumes of deionized water) C18 cartridge (bed weight 10 g) and eluted with a solution of 50-80% acetonitrile in water. The fractions containing the final product were collected, combined, and concentrated. The residue was further purified by silica gel column chromatography using chloroform:methanol:water=5:4:0.5 (by volume) as an eluant to obtain pure GM1 (105 mg, 97%) as a white powder. ¹H NMR (600 MHz, MeOD) δ 5.60 (dt, J=14.4, 7.8 Hz, 1H), 5.36 (dd, J=15.0, 7.8 Hz, 1H), 4.83 (d, J=9.0 Hz, 1H), 4.37 (d, J=7.8 Hz, 1H), 4.33 (d, J=7.8 Hz, 1H), 4.22 (d, J=7.8 Hz, 1H), 4.12-4.05 (m, 3H), 3.99 (t, J=8.4 Hz, 1H), 3.95-3.26 (m, 30H), 3.20 (t, J=8.4 Hz, 1H), 2.65 (dd, J=12.6, 4.8 Hz, 1H), 2.09 (t, J=7.8 Hz, 2H), 1.96-1.92 (m, 2H), 1.93 (s, 3H), 1.91 (s, 3H), 1.83 (t, J=12.0 Hz, 1H), 1.55-1.06 (m, 52H), 0.82 (t, J=6.6 Hz, 6H). ¹³C NMR (150 MHz, MeOD) δ 174.53, 174.23, 173.83, 173.41, 133.64, 129.99, 105.24, 103.55, 103.04, 102.71, 102.08, 81.63, 79.88, 77.62, 75.08, 75.03, 74.94, 74.71, 74.51, 74.21, 73.70, 73.44, 73.18, 71.98, 71.56, 71.10, 69.64, 69.05, 68.83, 68.51, 68.34, 68.29, 63.99, 61.59, 60.99, 60.39, 60.32, 53.30, 52.38, 51.34, 37.16, 35.97, 32.08, 31.71, 31.69, 29.49, 29.46, 29.43, 29.42, 29.38, 29.31, 29.25, 29.12, 29.09, 29.08, 29.04, 25.78, 22.38, 22.37, 22.35, 21.19, 13.08, 13.07. HRMS (ESI-Orbitrap) m/z: [M−H]⁻ calculated for C₇₃H₁₃₀N₃O₃₁ 1544.8694, found 1544.8661.

### Neu5Gc-GM1:

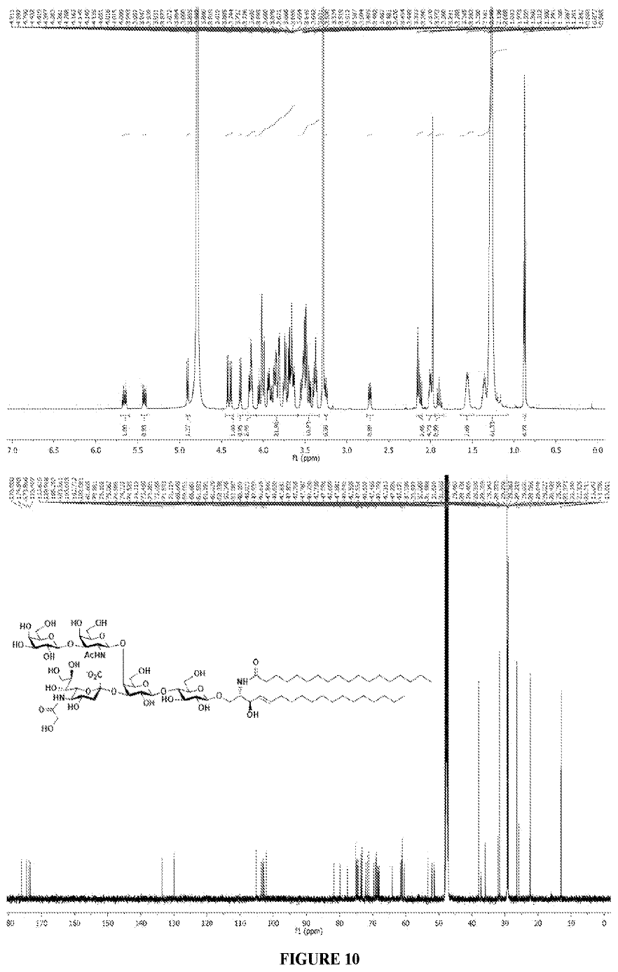

To a solution of Neu5Gc-GM1βSph (80 mg, 0.061 mmol) in sat. NaHCO₃-THF (3 mL, 2:1), stearoyl chloride (28 mg, 0.92 mmol, 1.5 eq) in 1 mL THF was added. The resulting mixture was stirred vigorously at room temperature for 2 hours. An additional 0.5 eq of stearoyl chloride was added and the mixture was stirred for another 2 hours and concentrated. The sample was loaded to a pre-conditioned C18 cartridge (bed weight 10 g) and eluted with a solution of 50-80% acetonitrile in water. The fractions containing the final product were collected, combined, and concentrated. The residue was further purified by silica gel column chromatography using chloroform:methanol:water=5:4:0.5 (by volume) as an eluant to obtain Neu5Gc-GM1 (94 mg, 98%) as a white powder. ¹H NMR (600 MHz, MeOD) δ 5.66 (dt, J=15.0, 7.2 Hz, 1H), 5.42 (dd, J=15.0, 7.2 Hz, 1H), 4.90 (d, J=8.4 Hz, 1H), 4.42 (d, J=8.4 Hz, 1H), 4.39 (d, J=8.4 Hz, 1H), 4.27 (d, J=7.8 Hz, 1H), 4.18-4.13 (m, 3H), 4.06-3.62 (m, 22H), 3.56-3.35 (m, 11H), 3.25 (t, J=7.8, 1H), 2.72 (dd, J=12.6, 5.4 Hz, 1H), 2.16-1.99 (m, 4H), 1.97 (s, 3H), 1.90 (t, J=12.0 Hz, 1H), 1.60-1.22 (m, 52H), 0.87 (t, J=6.6 Hz, 6H). ¹³C NMR (150 MHz, MeOD) δ 176.00, 174.55, 173.87, 173.41, 133.61, 129.97, 105.20, 103.56, 103.07, 102.71, 102.08, 81.60, 79.86, 77.59, 75.10, 75.06, 74.99, 74.72, 74.53, 74.21, 73.45, 73.21, 72.06, 71.59, 71.12, 69.65, 69.05, 68.86, 68.56, 68.35, 68.11, 64.02, 61.59, 61.19, 61.02, 60.43, 60.34, 53.35, 52.09, 51.33, 37.94, 37.24, 35.98, 32.06, 31.69, 31.67, 31.66, 29.49, 29.46, 29.43, 29.41, 29.39, 29.37, 29.34, 29.33, 29.29, 29.26, 29.22, 29.09, 29.07, 29.05, 29.02, 26.43, 25.76, 22.37, 22.34, 22.33, 22.31, 13.05, 13.04. HRMS (ESI-Orbitrap) m/z: [M−H]⁻ calculated for C₇₃H₁₃₀N₃O₃₂ 1560.8643, found 1560.8624. See FIG. 10 for ¹H and ¹³C NMR spectra of Neu5Gc-containing GM1 sphingosine (Neu5Gc-GM1βSph). See also FIG. 9 for ¹H and ¹³C NMR of Neu5Ac-containing GM1 (Neu5Ac-GM1).

### Results and Discussion

The production of target GM1 gangliosides (d18:1-18:0) was completed by installing the stearoyl chain to the amino group in GM1 sphingosines using stearoyl chloride in a mixed solvent of THF/aq. NaHCO₃(Scheme 4).

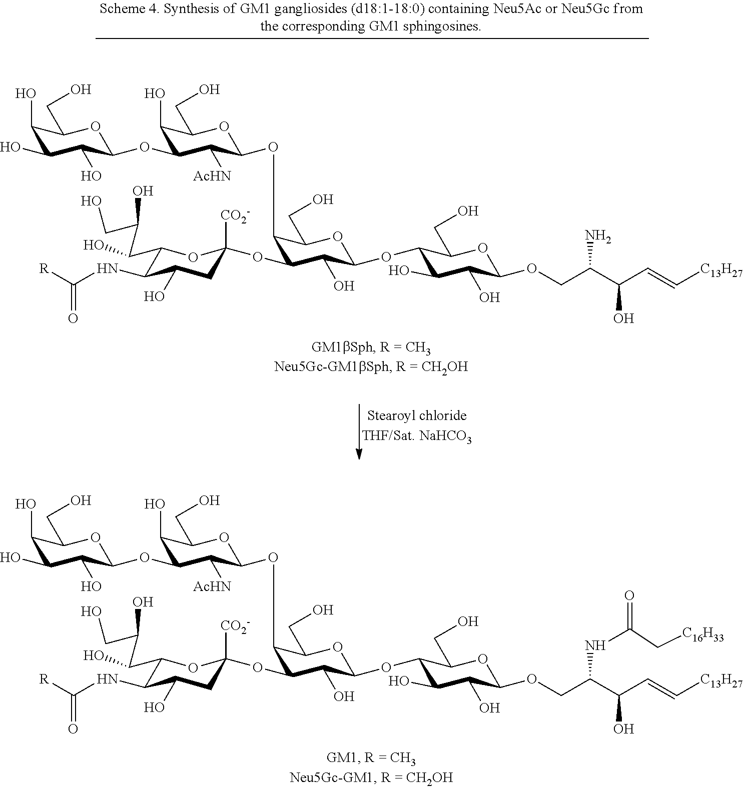

The acylation reaction progress was monitored by HRMS and reached completion in less than 4 hours. The reaction mixture was purified using a C18 cartridge then a silica gel column to obtain the desired gangliosides GM1 (97%) and Neu5Gc-GM1 (98%), respectively.

### Example 10: Sequential One-Pot Multienzyme (OPME) Synthesis of Ganglio-Series Ganglioside Glycosphingosines

To prepare a library of 0, a, b, and c-series of ganglioside glycosphingosines, a compound of each series was synthesized, including GM3βSph (lyso-GM3) for a-series, GD3βSph (lyso-GD3) for b-series, and GT3βSph (lyso-GT3) for c-series. In the case of 0-series ganglioside glycosphingosines, GM3βSph (lyso-GM3) was used as the starting material.

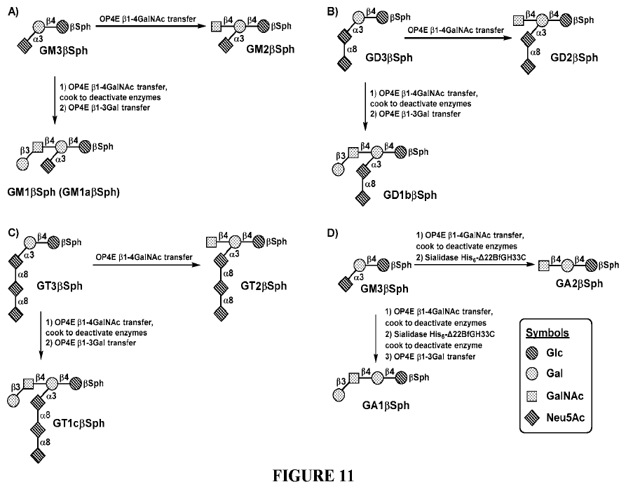

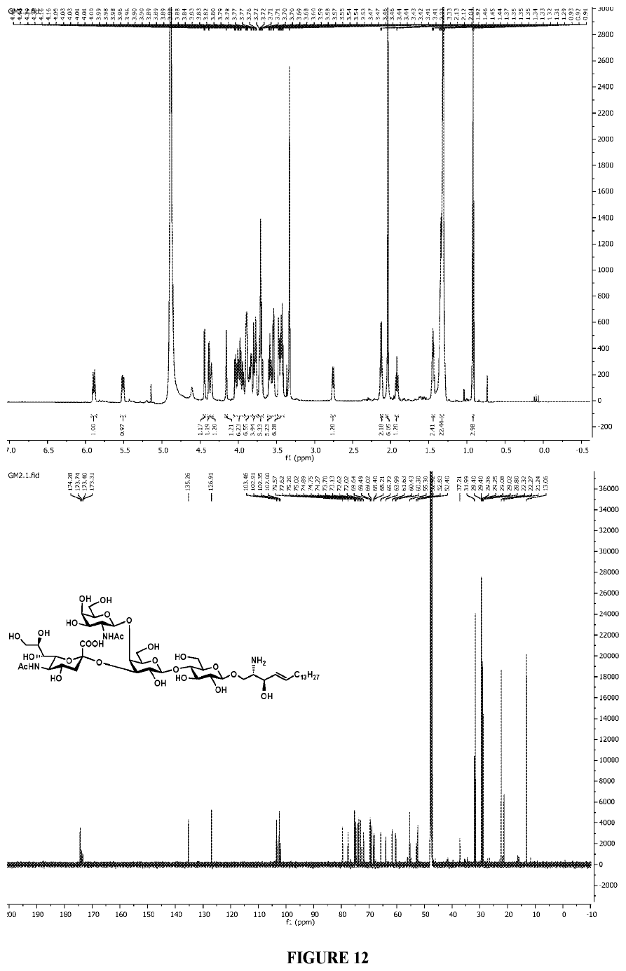

As shown in FIG. 11, a one-pot four-enzyme (OP4E) reaction containing BLNahK, PmGlmU, PmPpA, and MBP-Δ15CjCgtA-D4-Y238E in the presence of sodium cholate catalyzed the formation of GM2βSph (lyso-GM2) (FIG. 11, Panel A), GD2βSph (lyso-GD2) (FIG. 11, Panel B), and GT2βSph (lyso-GT2) (FIG. 11, Panel C) in excellent yields from GM3βSph (lyso-GM3), GD3βSph (lyso-GD3), and GT3βSph (lyso-GT3), respectively.

A Multistep One-pot Multienzyme (MSOPME) reaction process was used to produce GM1βSph (lyso-GM1) (FIG. 11, Panel A), GD13Sph (lyso-GD1) (FIG. 11, Panel B), and GT1βSph (lyso-GT1) (FIG. 11, Panel C) from GM3βSph (lyso-GM3), GD3βSph (lyso-GD3), and GT3βSph (lyso-GT3), respectively. In the first step, the reactions were carried out as described above for the synthesis of GM2βSph (lyso-GM2), GD2βSph (lyso-GD2), and GT2βSph (lyso-GT2) from GM3βSph (lyso-GM3), GD3βSph (lyso-GD3), and GT3 βSph (lyso-GT3), respectively. Without purification, the reaction mixture was incubated in a boiling water bath for 10 minutes to deactivate enzymes, cooled down, and then used for the next OP4E reaction step by adding SpGalK, BLUSP, PmPpA, and MBP-CjCgtBΔ30-His₆ to produce the targets GM1βSph (lyso-GM1), GD13Sph (lyso-GD1), and GT1βSph (lyso-GT1), respectively, in excellent yields.

For the synthesis of 0-series ganglioside glycosphingosines, another Multistep One-pot Multienzyme (MSOPME) reaction process was used. In the first step, GM2βSph (lyso-GM2) was synthesized from GM3βSph (lyso-GM3) using the OP4E reaction containing BLNahK, PmGlmU, PmPpA, and MBP-Δ15CjCgtA-D4-Y238E in the presence of sodium cholate. Without purification, the reaction mixture was incubated in a boiling water bath for 10 min to deactivate enzymes, cooled down, and incubated with a recombinant sialidase His₆-Δ22BfGH33C (the second step) to produce GA2βSph (lyso-GA2) (FIG. 11, Panel D). The product was purified by a C18-cartridge to obtain pure GA2βSph (lyso-GA2). Alternatively, without purification the reaction mixture containing the GA2βSph (lyso-GA2) product was incubated in a boiling water bath for 10 minutes to deactivate enzymes, cooled down, and used for another OP4E (the third step) reaction by adding SpGalK, BLUSP, PmPpA, and MBP-CjCgtBΔ30-His₆ to produce GA1βSph (lyso-GA1) after C18-cartridge purification (FIG. 11, Panel D).

Synthesis of GM2βSph from GM3βSph:

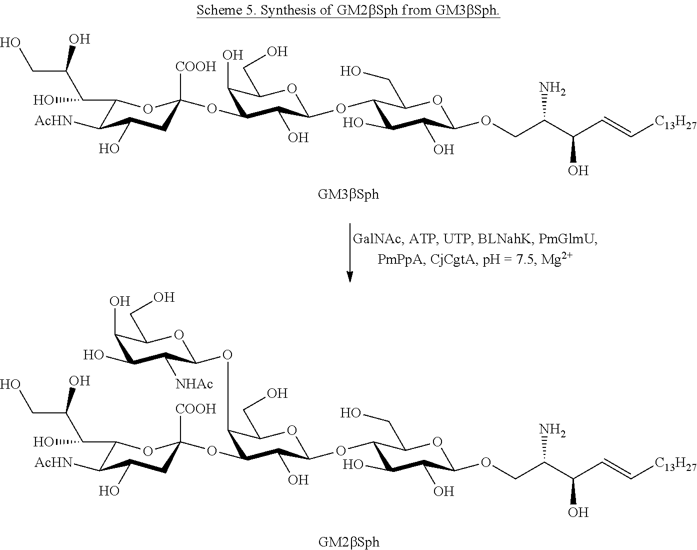

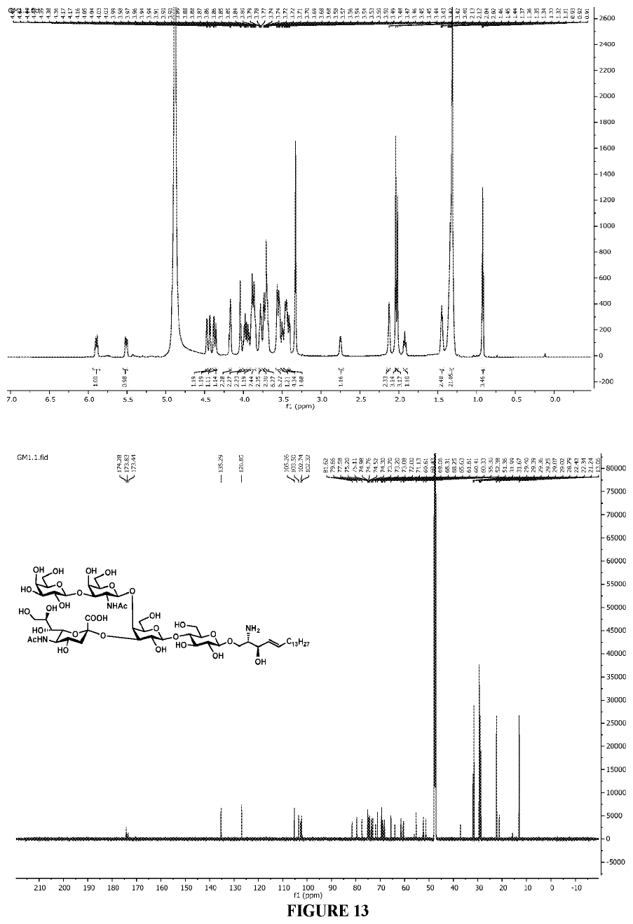

As shown in Scheme 5, GM2βSph was synthesized from GM3βSph. A reaction mixture containing GM3βSph (50 mg, 0.055 mmol), GalNAc (24 mg, 0.11 mmol), sodium cholate (10 mM), MgCl₂ (20 mM), ATP (60.6 mg, 0.11 mmol),UTP (58.0 mg, 0.11 mmol) BLNahK (1.0 mg), PmGlmU (1.0 mg), CjCgtA (1.50 mg), and PmPpA (0.5 mg) in 5 mL of Tris-HCl buffer (100 mM, pH 7.5) was incubated at 30° C. with agitation at 100 rpm. The product formation was monitored by FIRMS. After 48 hours, the HRMS indicated that the GM3 was almost consumed. Then the reaction was quenched by adding the same volume (5 mL) of ice-cold ethanol. The mixture was incubated at 4° C. for 30 minutes and centrifuged to remove precipitates. The supernatant was concentrated and the residue was purified using a 51 g ODS-SM column (50 μM, 120 Å, Yamazen) on a CombiFlash® Rf 200i system. After loading the sample, the column was washed with water for 5 minutes and the product was eluted with 50% acetonitrile in water (v/v). The whole process took about 25 minutes. The fractions containing product were collected, concentrated and the residue was purified by silica gel column chromatography. A mixed solvent CH₂Cl₂:methanol=5:2 (by volume) was used to remove sodium cholate and CH₂Cl₂:methanol:water=5:4:1 (by volume) was used as an eluent to obtain pure GM2βSph. The fractions containing pure product were collected, concentrated and lyophilized to obtain GM2βSph as a white powder (58 mg, 95% yield). ¹H NMR (800 MHz, Methanol-d4) δ 5.89 (dt, J=14.4, 6.8 Hz, 1H), 5.51 (dd, J=15.4, 6.6 Hz, 1H), 4.44 (d, J=7.9 Hz, 1H), 4.38 (d, J=7.8 Hz, 1H), 4.35 (t, J=5.8 Hz, 1H), 4.16 (d, J=3.2 Hz, 1H), 4.06-3.92 (m, 6H), 3.92-3.81 (m, 7H), 3.81-3.75 (m, 4H), 3.73-3.67 (m, 5H), 3.61-3.52 (m, 5H), 3.49-3.40 (m, 6H), 2.76 (dd, J=12.7, 4.9 Hz, 1H), 2.14-2.11 (m, 2H), 2.04 (s, 6H), 1.92 (t, J=12.0 Hz, 1H), 1.46-1.43 (m, 2H), 1.31 (d, J=7.6 Hz, 22H), 0.92 (d, J=7.2 Hz, 3H). ¹³C NMR (200 MHz, Methanol-d4) δ 174.3, 173.7, 173.4, 173.3, 135.3, 126.9, 103.5, 102.9, 102.3, 102.0, 79.6, 77.6, 75.2, 75.0, 74.9, 74.7, 74.3, 73.7, 73.1, 72.6, 72.0, 69.6, 69.5, 69.0, 68.4, 68.2, 65.7, 64.0, 61.6, 60.4, 60.3, 55.3, 52.9, 52.8, 52.4, 37.2, 32.0, 29.5, 29.4, 29.3, 29.2, 29.1, 29.0, 28.8, 22.4, 22.3, 21.2, 13.1. FIG. 12 shows ¹H and ¹³C NMR spectra of GM2βSph (d18:1).

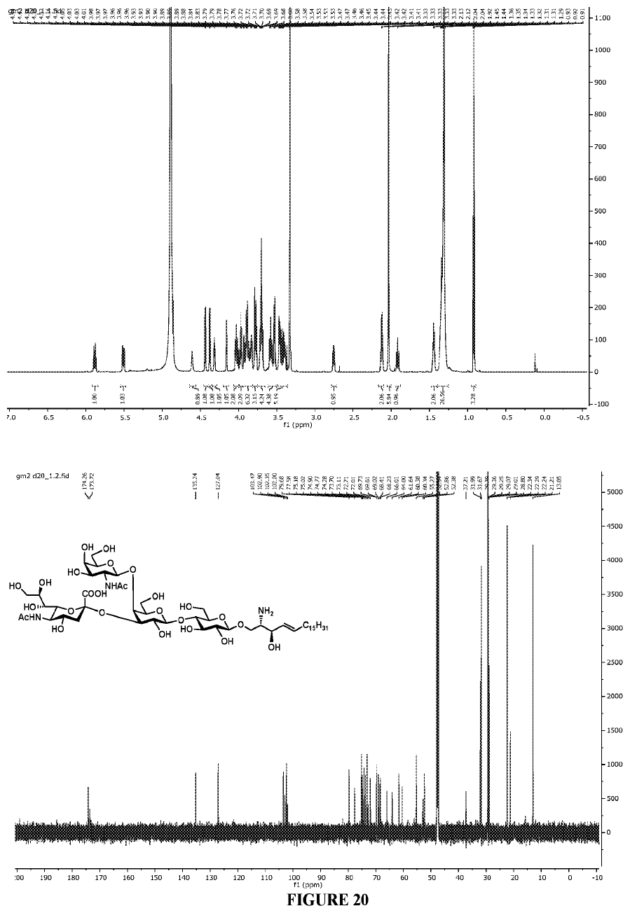

Using the same procedure, GM2βSph (d20:1) was synthesized from GM2βSph (d20:1). ¹H NMR (800 MHz, Methanol-d4) δ 5.88 (dtd, J=15.0, 6.8, 1.2 Hz, 1H), 5.51 (ddd, J=15.4, 6.8, 1.5 Hz, 1H), 4.61 (s, 1H), 4.43 (d, J=7.9 Hz, 1H), 4.38 (d, J=7.8 Hz, 1H), 4.32 (t, J=5.9 Hz, 1H), 4.16 (d, J=3.2 Hz, 1H), 4.06-4.00 (m, 2H), 3.99-3.94 (m, 2H), 3.94-3.82 (m, 6H), 3.82-3.75 (m, 3H), 3.75-3.66 (m, 4H), 3.61-3.51 (m, 4H), 3.49-3.36 (m, 5H), 2.76 (dd, J=12.7, 4.9 Hz, 1H), 2.12 (q, J=7.6 Hz, 2H), 2.04 (d, J=1.7 Hz, 6H), 1.92 (t, J=12.0 Hz, 1H), 1.44 (q, J=7.4 Hz, 2H), 1.40-1.25 (m, 27H), 0.92 (t, J=7.1 Hz, 3H).; ¹³C NMR (200 MHz, Methanol-d4) δ 174.3, 173.7, 135.2, 127.0, 103.5, 102.9, 102.3, 102.0, 79.7, 77.6, 75.2, 75.0, 74.9, 74.8, 74.3, 73.7, 73.1, 72.7, 72.0, 69.7, 69.6, 69.0, 68.4, 68.2, 66.0, 64.0, 61.6, 60.4, 60.3, 55.3, 52.9, 52.8, 52.4, 37.2, 32.0, 31.7, 29.5, 29.4, 29.2, 29.1, 29.0, 28.8, 22.4, 22.3, 22.2, 21.2, 13.1. FIG. 20 shows ¹H and ¹³C NMR spectra of GM2βSph (d20:1).

Synthesis of GM1βSph from GM3βSph:

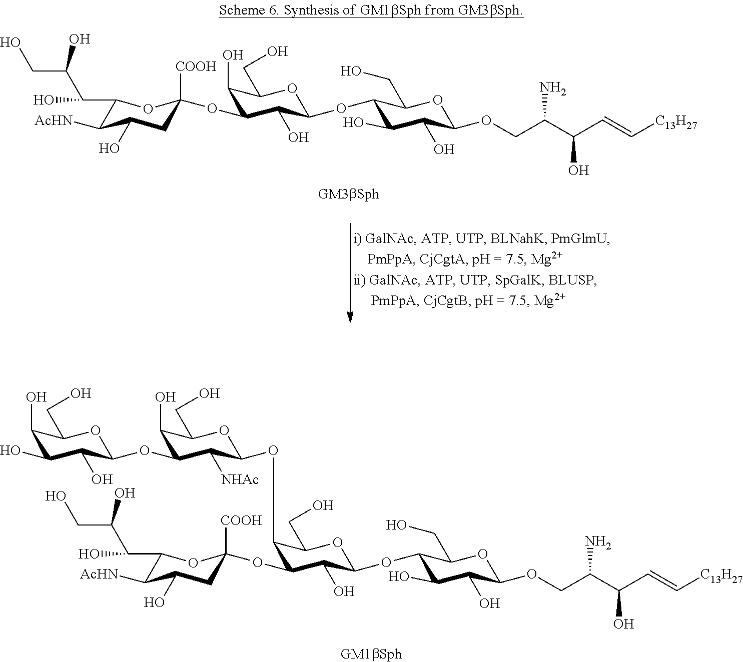

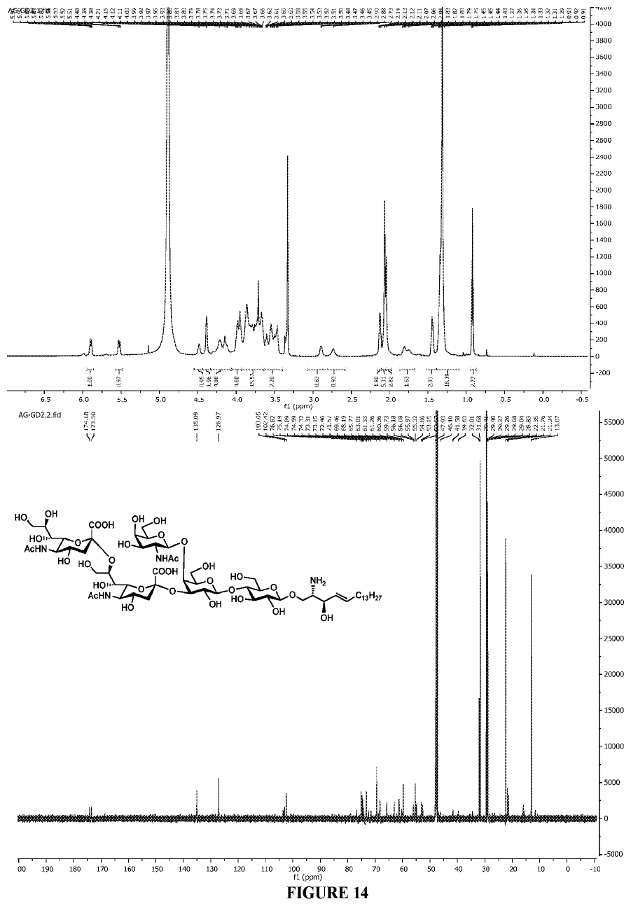

As shown in Scheme 6, GM1βSph was synthesized from GM3βSph. GM3βSph (500 mg, 0.55 mmol), GalNAc (240 mg, 1.10 mmol), sodium cholate (10 mM), ATP (606 mg, 1.10 mmol), and UTP (580 mg, 1.10 mmol) were incubated in 50 mL of Tris-HCl buffer (100 mM, pH 7.5) containing BLNahK (10.0 mg), PmGlmU (10.0 mg), CjCgtA (15 mg), and PmPpA (5.0 mg). The reaction was carried out by incubating the solution in an incubator shaker at 30° C. with agitation at 100 rpm. The product formation was monitored by HRMS. After 48 hours HRMS indicated that the GM3βSph was almost consumed. Then, the reaction mixture was incubated in a boiling water bath at 100° C. for 10 minutes and allowed to come to room temperature. In the same reaction container, galactose (198 mg, 1.10 mmol), ATP (606 mg, 1.10 mmol), UTP (580 g, 1.10 mmol), SpGalK (12.0 mg), BLUSP (12.0 mg), CjCgtB (18 mg), and PmPpA (6.0 mg) were added. The pH of the reaction mixture (60 mL) was adjusted to 7.5 by adding NaOH (4 M) and incubated in a shaker at 30° C. by agitating at 100 rpm. The product formation was monitored by HRMS. After 48 hours, HRMS indicated that the GM2βSph was almost consumed. Prechilled ethanol (60 mL) was added and the mixture was incubated at 4° C. for 30 minutes. The precipitates were removed by centrifugation, the supernatant was concentrated, and one third of the residue was purified using a 51 g ODS-SM column (50 μM, 120 Å, Yamazen) on a CombiFlash® Rf 200i system. After loading the sample, the column was washed with water for 5 minutes and the product was eluted with 45% acetonitrile in water (v/v). The whole process took about 20 minutes. The same purification process was repeated to purify the product from the other two-thirds of the sample. The fractions containing product were collected, concentrated, and the residue was purified by silica gel column chromatography. A mixed solvent system of CH₂Cl₂:methanol=5:2 (by volume) was used to remove sodium cholate and CH₂Cl₂:methanol:water=5:4:1 (by volume) was used as an eluent to obtain pure GM2βSph. The fractions containing pure product were collected, concentrated, and lyophilized to obtain the final pure GM1βSph as a white powder (660 mg, 95% yield). ¹H NMR (800 MHz, Methanol-d₄) δ 5.89 (dt, J=14.5, 6.8 Hz, 1H), 5.51 (dd, J=15.3, 6.7 Hz, 1H), 4.47 (d, J=7.6 Hz, 1H), 4.43 (d, J=7.9 Hz, 1H), 4.38 (d, J=7.8 Hz, 1H), 4.36 (t, J=5.7 Hz, 1H), 4.17 (dd, J=10.9, 6.7 Hz, 2H), 4.04 (dd, J=10.5, 3.1 Hz, 2H), 4.00-3.96 (m, 2H), 3.94 (dd, J=11.5, 3.4 Hz, 1H), 3.91-3.84 (m, 7H), 3.78 (dd, J=12.2, 7.7 Hz, 2H), 3.74 (d, J=6.1 Hz, 2H), 3.70 (dq, J=18.2, 10.5, 7.5 Hz, 6H), 3.55 (dt, J=21.3, 6.3 Hz, 6H), 3.50 (dd, J=9.8, 3.2 Hz, 1H), 3.48-3.43 (m, 4H), 3.41 (d, J=8.9 Hz, 1H), 2.75 (dd, J=13.0, 4.9 Hz, 1H), 2.16-2.11 (m, 2H), 2.04 (s, 3H), 2.02 (s, 3H), 1.92 (t, J=11.9 Hz, 1H), 1.44 (q, J=7.4 Hz, 2H), 1.34-1.30 (m, 21H), 0.92 (t, J=7.1 Hz, 3H). ¹³C NMR (200 MHz, Methanol-d₄) δ 174.3, 173.8, 173.4, 135.3, 126.9, 105.3, 103.5, 102.7, 102.3, 102.0, 81.6, 79.7, 77.6, 75.2, 75.1, 75.0, 74.8, 74.5, 74.3, 73.7, 73.2, 73.1, 72.0, 71.1, 69.6, 69.4, 69.1, 68.8, 68.3, 68.2, 65.6, 64.0, 61.6, 61.0, 60.4, 60.3, 55.3, 52.4, 51.4, 51.4, 37.2, 32.0, 31.7, 29.5, 29.4, 29.3, 29.2, 29.1, 29.0, 28.8, 22.4, 22.3, 21.2, 13.1. FIG. 13 shows ¹H and ¹³C NMR spectra of GM1βSph (d18:1).

Using the same procedure, GM1βSph (d20:1) was synthesized from GM3βSph (d20:1).

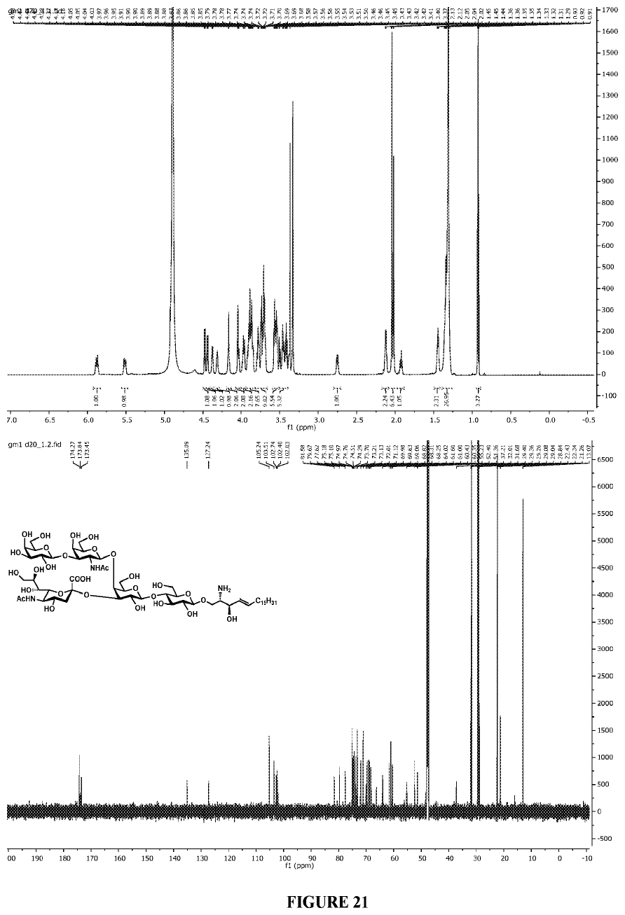

¹H NMR (800 MHz, Methanol-d₄) δ 5.88 (dt, J=14.5, 6.8 Hz, 1H), 5.51 (dd, J=15.4, 6.8 Hz, 1H), 4.47 (d, J=7.6 Hz, 1H), 4.44 (d, J=7.9 Hz, 1H), 4.38 (d, J=7.8 Hz, 1H), 4.31 (t, J=5.8 Hz, 1H), 4.16 (d, J=3.0 Hz, 2H), 4.07-4.02 (m, 2H), 3.97 (d, J=11.0 Hz, 2H), 3.94-3.82 (m, 8H), 3.81-3.66 (m, 10H), 3.56 (dt, J=23.0, 7.8 Hz, 6H), 3.51-3.40 (m, 5H), 2.75 (dd, J=12.7, 4.9 Hz, 1H), 2.12 (q, J=7.3 Hz, 2H), 2.03 (d, J=17.1 Hz, 6H), 1.92 (t, J=12.0 Hz, 1H), 1.45 (p, J=7.3 Hz, 2H), 1.31 (d, J=8.3 Hz, 27H), 0.92 (t, J=7.2 Hz, 3H). ¹³C NMR (200 MHz, Methanol-d₄) δ 174.3, 173.8, 173.4, 135.1, 127.2, 105.2, 103.5, 102.7, 102.4, 102.0, 81.6, 79.7, 77.6, 75.2, 75.1, 75.0, 74.8, 74.5, 74.3, 73.7, 73.2, 73.1, 72.0, 71.1, 70.0, 69.6, 69.1, 68.8, 68.3, 68.2, 66.3, 64.0, 61.6, 61.0, 60.4, 60.3, 55.2, 52.4, 51.4, 48.5, 37.2, 32.0, 31.7, 29.5, 29.4, 29.3, 29.1, 29.0, 28.8, 22.4, 22.3, 21.3, 13.1. FIG. 21 shows ¹H and ¹³C NMR spectra of GM1βSph (d20:1).

Synthesis of GD2βSph from GD3βSph:

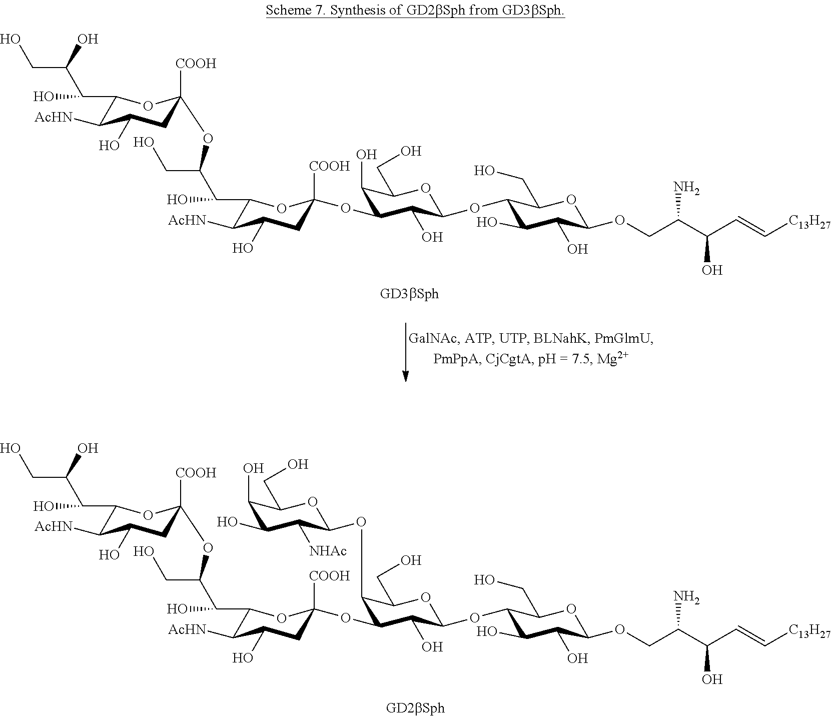

As shown in Scheme 7, GD2βSph was synthesized from GD3βSph. A reaction mixture containing GD3βSph (50 mg, 0.041 mmol), GalNAc (18.1 mg, 0.08 mmol), ATP (45.1 mg, 0.08 mmol), UTP (43.3 mg, 0.08 mmol), sodium cholate (10 mM), BLNahK (0.8 mg), PmGlmU (0.8 mg), CjCgtA (1.50 mg), and PmPpA (0.4 mg) in 4 mL of Tris-HCl buffer (100 mM, pH 7.5) was incubated in an incubator shaker at 30° C. for 48 hours with agitation at 100 rpm. The product formation was monitored by HRMS. After 48 hours, the HRMS indicated that the GD3 was almost consumed. Then the reaction was quenched by adding the same volume (4 mL) of ice-cold ethanol. The mixture was incubated at 4° C. for 30 minutes and centrifuged to remove precipitates. The supernatant was concentrated and the residue was purified using a 37 g ODS-SM column (50 M, 120 Å, Yamazen) on a CombiFlash® Rf 200i system. After loading the sample, the column was washed with water for 5 minutes and the product was eluted with 45% acetonitrile in water (v/v). The whole process took about 25 minutes. The fractions containing pure product were collected and concentrated. The residue was purified by silica gel column chromatography. A mixed solvent system of CH₂Cl₂:methanol=5:2 (by volume) was used to remove sodium cholate and CH₂Cl₂:methanol:water=5:4:1 (by volume) was used as an eluent to obtain pure GD2βSph (d18:1) as a white powder (55.5 mg, 95% yield). ¹H NMR (800 MHz, Methanol-d₄) δ 5.90 (dt, J=14.4, 6.8 Hz, 1H), 5.53 (dd, J=15.3, 6.5 Hz, 1H), 4.48 (d, J=7.8 Hz, 1H), 4.41-4.37 (m, 2H), 4.20 (d, J=11.0 Hz, 1H), 4.16 (d, J=8.8 Hz, 1H), 4.11 (d, J=14.4 Hz, 1H), 4.00 (t, J=10.0 Hz, 1H), 3.95 (s, 2H), 3.86 (t, J=14.4 Hz, 6H), 3.83-3.78 (m, 4H), 3.78-3.70 (m, 7H), 3.68 (d, J=6.6 Hz, 2H), 3.65-3.62 (m, 1H), 3.62-3.57 (m, 2H), 3.57-3.52 (m, 3H), 3.52-3.45 (m, 4H), 3.37-3.34 (m, 1H), 2.88 (d, J=12.0 Hz, 1H), 2.71 (s, 1H), 2.13 (q, J=7.1 Hz, 2H), 2.10-2.06 (m, 3H), 2.04 (s, 6H), 1.82 (d, J=38.9 Hz, 2H), 1.45 (p, J=7.4 Hz, 2H), 1.39-1.28 (m, 21H), 0.92 (t, J=7.1 Hz, 3H). ¹³C NMR (200 MHz, Methanol-d₄) δ 174.2, 173.5, 135.1, 127.0, 103.0, 102.4, 76.8, 75.2, 74.9, 74.6, 74.3, 73.3, 73.2, 72.5, 71.6, 69.5, 68.2, 65.8, 63.0, 61.4, 61.3, 60.4, 59.7, 56.2, 56.1, 56.0, 55.3, 54.9, 53.1, 52.7, 47.9, 46.1, 41.6, 39.6, 32.0, 31.7, 29.5, 29.4, 29.3, 29.2, 29.1, 29.0, 28.8, 22.3, 21.8, 21.3, 13.1. FIG. 14 shows ¹H and ¹³C NMR spectra of GD2βSph (d18:1).

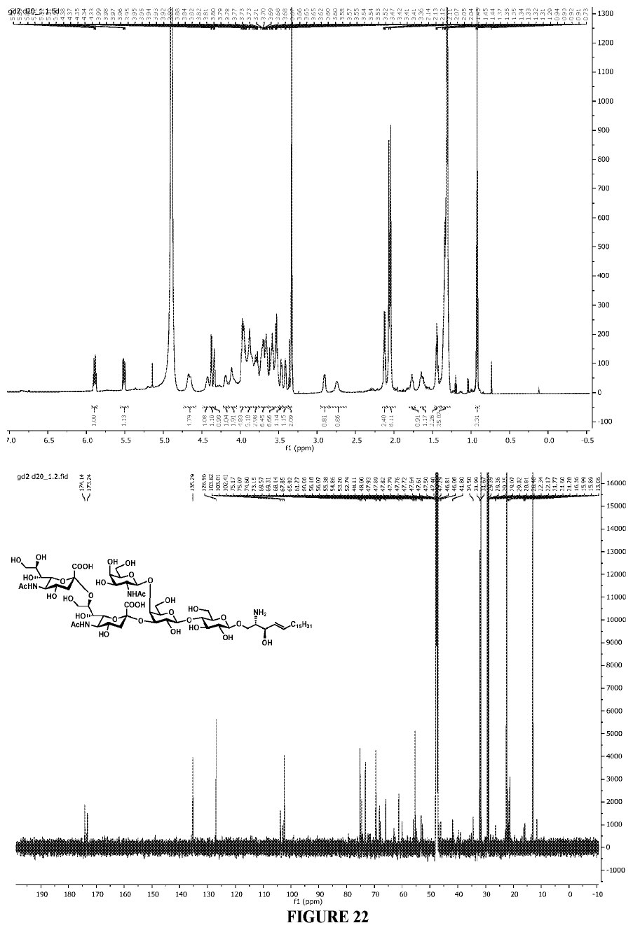

Using the same procedure, GD2βSph (d20:1) was synthesized from GD3βSph (d20:1). ¹H NMR (800 MHz, Methanol-d₄) δ 5.89 (dt, J=16.0, 6.8 Hz, 1H), 5.51 (ddt, J=15.5, 6.8, 1.7 Hz, 1H), 4.73-4.58 (m, 2H), 4.42 (s, 1H), 4.37 (d, J=7.8 Hz, 1H), 4.34 (t, J=5.8 Hz, 1H), 4.22-4.16 (m, 1H), 4.14-4.05 (m, 2H), 3.96 (dtd, J=15.0, 11.4, 5.9 Hz, 5H), 3.88 (q, J=28.0 Hz, 5H), 3.83-3.76 (m, 3H), 3.76-3.63 (m, 6H), 3.63-3.49 (m, 7H), 3.46 (dt, J=9.8, 3.6 Hz, 1H), 3.41 (dt, J=8.6, 4.2 Hz, 1H), 3.37 (d, J=3.4 Hz, 2H), 2.91 (dd, J=12.9, 4.7 Hz, 1H), 2.74 (d, J=13.5 Hz, 1H), 2.12 (q, J=7.3 Hz, 2H), 2.09-1.98 (m, 8H), 1.77 (t, J=11.2 Hz, 1H), 1.66 (s, 1H), 1.47-1.41 (m, 2H), 1.39-1.28 (m, 25H), 0.92 (t, J=7.1 Hz, 3H). ¹³C NMR (200 MHz, Methanol-d₄) δ 174.1, 173.2, 135.3, 127.0, 103.8, 103.0, 102.4, 75.2, 75.1, 74.6, 73.1, 69.6, 69.3, 68.1, 67.9, 65.9, 61.3, 60.1, 56.2, 56.1, 56.0, 55.4, 54.9, 53.2, 52.7, 48.1, 48.0, 47.9, 47.9, 47.8, 47.8, 47.8, 47.7, 47.6, 47.6, 47.5, 47.4, 47.3, 46.8, 46.1, 41.8, 34.5, 32.0, 31.7, 29.4, 29.4, 29.3, 29.1, 29.0, 28.8, 26.5, 22.9, 22.3, 22.2, 21.8, 21.6, 21.3, 16.4, 16.0, 15.9, 13.1. FIG. 22 shows ¹H and ¹³C NMR spectra of GD2βSph (d20:1).

Synthesis of GD2βSph from GD3βSph:

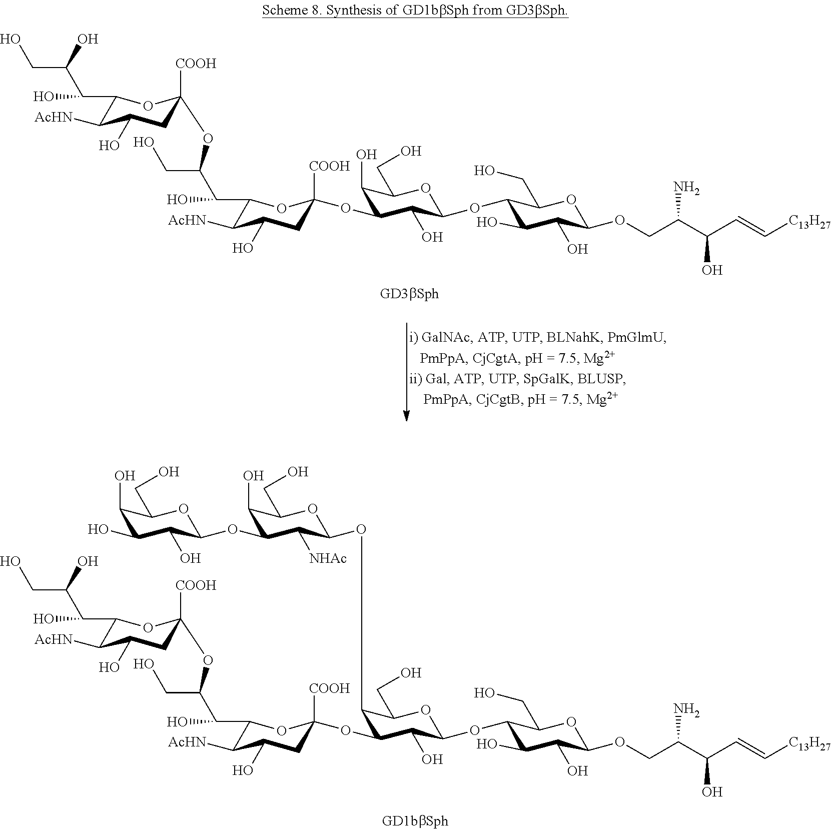

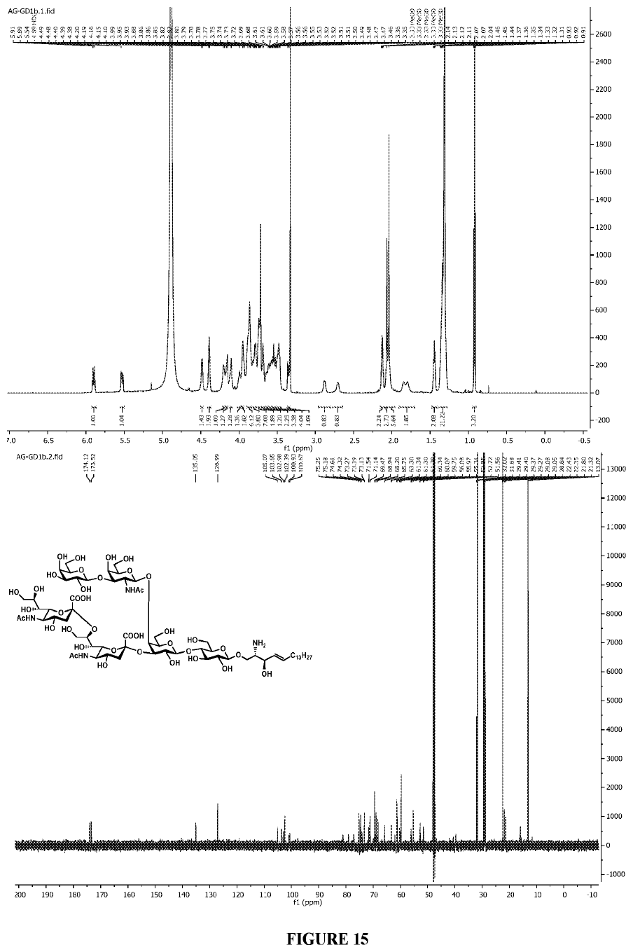

As shown in Scheme 8, GD1bβSph was synthesized from GD3βSph. A reaction mixture containing GD3βSph (250 mg, 0.21 mmol), GalNAc (91.50 mg, 0.41 mmol), ATP (225.91 mg, 0.41 mmol), UTP (216.48 mg, 0.41 mmol), sodium cholate (10 mM), BLNahK (4.0 mg), PmGlmU (4.0 mg), CjCgtA (7.0 mg), and PmPpA (2.0 mg) in 20 mL of Tris-HCl buffer (100 mM, pH 7.5) was incubated in an incubator shaker at 30° C. with agitation at 100 rpm. The product formation was monitored by FIRMS. After 48 hours, FIRMS indicated that the GD3βSph was almost consumed. Then the reaction mixture was incubated in a boiling water bath at 100° C. for 10 minutes and allowed to come to room temperature. In the same reaction container, galactose (73.8 mg, 0.41 mmol), ATP (226.0 mg, 0.41 mmol), UTP (216.50 g, 0.41 mmol), SpGalK (5.0 mg), BLUSP (5.0 mg), CjCgtB (8 mg), and PmPpA (2.5 mg) were added. The pH of the reaction mixture (25 mL) was adjusted to 7.5 by adding NaOH (4 M) and incubated in a shaker at 30° C. by agitating at 100 rpm. The product formation was monitored by FIRMS. After 48 hours, HRMS indicated that the GD2βSph was almost consumed. Prechilled ethanol (25 mL) was added and the mixture was incubated at 4 ° C. for 30 minutes. The precipitates were removed by centrifugation, the supernatant was concentrated, and the residue was purified using a 51 g ODS-SM column (50 M, 120 Å, Yamazen) on a CombiFlash® Rf 200i system. After loading the sample, the column was washed with water for 5 minutes and the product was eluted with 40% acetonitrile in water (v/v). The whole process took about 30 minutes. The fractions containing the product were collected, concentrated, and the residue was purified by silica gel column chromatography. A mixed solvent system of CH₂Cl₂:methanol=5:2 (by volume) was used to remove sodium cholate and CH₂Cl₂:methanol:water=5:4:1 (by volume) was used as an eluent to obtain the final pure GD1bβSph. The fractions containing the pure product were collected, concentrated, and lyophilized to obtain GD1bβSph (d18:1) as a white powder (305 mg, 95% yield). ¹H NMR (800 MHz, Methanol-d₄) δ 5.90 (dt, J=14.4, 6.8 Hz, 1H), 5.53 (dd, J=15.3, 6.5 Hz, 1H), 4.48 (d, J=7.8 Hz, 1H), 4.41-4.37 (m, 2H), 4.20 (d, J=11.0 Hz, 1H), 4.16 (d, J=8.8 Hz, 1H), 4.11 (d, J=14.4 Hz, 1H), 4.00 (t, J=10.0 Hz, 1H), 3.95 (s, 2H), 3.86 (t, J=14.4 Hz, 6H), 3.83-3.78 (m, 4H), 3.78-3.70 (m, 7H), 3.68 (d, J=6.6 Hz, 2H), 3.65-3.62 (m, 1H), 3.62-3.57 (m, 2H), 3.57-3.52 (m, 3H), 3.52-3.45 (m, 4H), 3.37-3.34 (m, 1H), 2.88 (d, J=12.0 Hz, 1H), 2.71 (s, 1H), 2.13 (q, J=7.1 Hz, 2H), 2.10-2.06 (m, 3H), 2.04 (s, 6H), 1.82 (d, J=38.9 Hz, 2H), 1.45 (p, J=7.4 Hz, 2H), 1.39-1.28 (m, 21H), 0.92 (t, J=7.1 Hz, 3H). ¹³C NMR (200 MHz, Methanol-d₄) δ 174.1, 173.5, 135.1, 127.0, 105.1, 103.7, 103.0, 102.4, 100.9, 100.7, 79.1, 75.3, 75.2, 74.6, 74.3, 74.1, 73.3, 73.2, 73.1, 71.5, 71.1, 69.5, 68.9, 68.2, 65.8, 63.3, 62.0, 61.4, 61.3, 61.2, 60.3, 60.1, 59.8, 56.2, 56.1, 56.0, 55.3, 52.8, 52.7, 51.6, 32.0, 31.7, 29.5, 29.4, 29.3, 29.2, 29.1, 29.0, 28.8, 22.4, 22.3, 21.8, 21.3, 13.1. FIG. 15 shows ¹H and ¹³C NMR spectra of GD1bβSph (d18:1).

Synthesis of GT2βSph from GT3βSph:

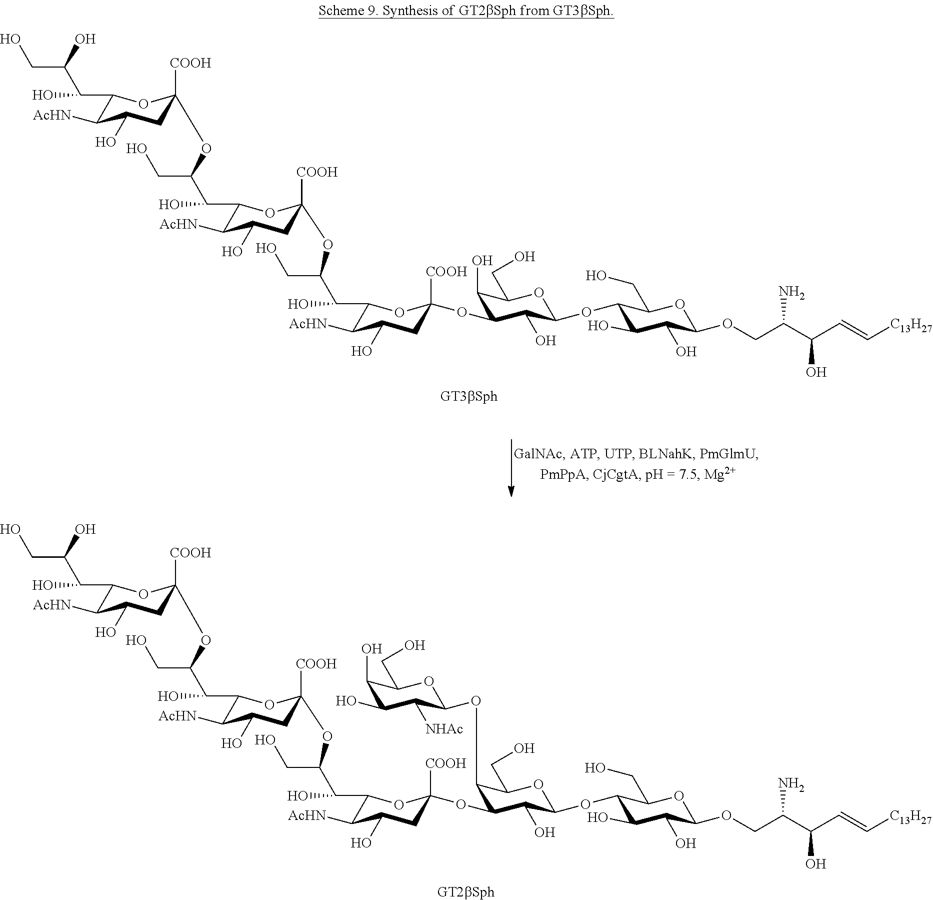

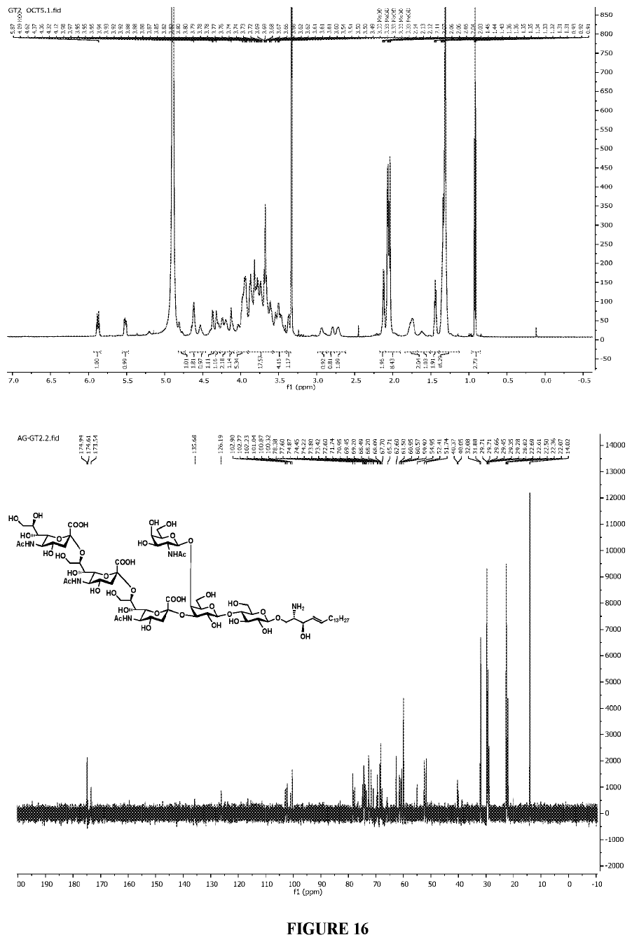

As shown in Scheme 9, GT2P3Sph was synthesized from GT3P3Sph. A reaction 5 mixture containing GT3P3Sph (50 mg, 0.03 mmol), GalNAc (14.8 mg, 0.07 mmol), ATP (36.4 mg, 0.07 mmol), UTP (34.8 mg, 0.07 mmol), sodium cholate (10 mM), BLNahK (0.8 mg), PmGlmU (0.8 mg), CjCgtA (1.50 mg), and PmPpA (0.4 mg) in 4 mL of Tris-HCl buffer (100 mM, pH 7.5) was incubated in an incubator shaker at 30° C. with agitation at 100 rpm. The product formation was monitored by FIRMS. After 48 hours, HRMS indicated that the GT3βSph was almost consumed and the reaction was quenched by adding the same volume (4 mL) of ice-cold ethanol. The mixture was incubated at 4° C. for 30 minutes and centrifuged to remove precipitates. The supernatant was concentrated and the residue was purified using a 37 g ODS-SM column (50 M, 120 Å, Yamazen) on a CombiFlash® Rf 200i system. After loading the sample, the column was washed with water for 5 minutes and the product was eluted with 35% acetonitrile in water (v/v). The whole process took about 25 minutes. The fractions containing product were collected, concentrated, and the residue was purified by silica gel column chromatography. A mixed solvent system of CH₂Cl₂:methanol=5:2 (by volume) was used to remove sodium cholate and CH₂Cl₂:methanol:water=5:4:1 (by volume) was used as an eluent to obtain the final pure GT2βSph. The fractions containing the pure product were collected, concentrated, and lyophilized to obtain GT2βSph as a white powder (53 mg, 95% yield). ¹H NMR (800 MHz, Methanol-d₄) δ 5.87 (dq, J=15.7, 8.9, 7.8 Hz, 1H), 5.52 (dd, J=15.5, 6.7 Hz, 1H), 4.62 (s, 2H), 4.53 (s, 1H), 4.39-4.33 (m, 1H), 4.31 (d, J=22.3 Hz, 1H), 4.27-4.15 (m, 2H), 4.11 (d, J=16.3 Hz, 1H), 4.05-3.91 (m, 5H), 3.90-3.57 (m, 18H), 3.58-3.40 (m, 4H), 3.40-3.34 (m, 1H), 2.92 (d, J=31.2 Hz, 1H), 2.79 (d, J=9.9 Hz, 1H), 2.73 (d, J=13.1 Hz, 1H), 2.12 (q, J=7.3 Hz, 2H), 2.10-1.90 (m, 8H), 1.76 (d, J=27.8 Hz, 2H), 1.62 (s, 1H), 1.44 (p, J=7.2 Hz, 2H), 1.40-1.13 (m, 16H), 0.92 (t, J=7.1 Hz, 3H). ¹³C NMR (200 MHz, Deuterium Oxide) δ 174.9, 174.6, 173.5, 135.7, 126.2, 102.9, 102.8, 102.2, 101.0, 100.9, 100.3, 78.4, 77.6, 74.9, 74.5, 74.2, 73.8, 73.4, 72.6, 71.7, 71.0, 69.4, 69.2, 68.5, 68.2, 68.1, 67.7, 65.7, 62.6, 61.5, 61.0, 60.6, 59.9, 55.0, 52.4, 51.7, 40.4, 40.1, 32.1, 31.9, 29.8, 29.7, 29.6, 29.4, 29.3, 29.2, 28.8, 22.7, 22.6, 22.5, 22.4, 22.1, 14.0. FIG. 16 shows ¹H and ¹³C NMR spectra of GT2βSph (d18:1).

Synthesis of GT1cβSph from GT3βSph:

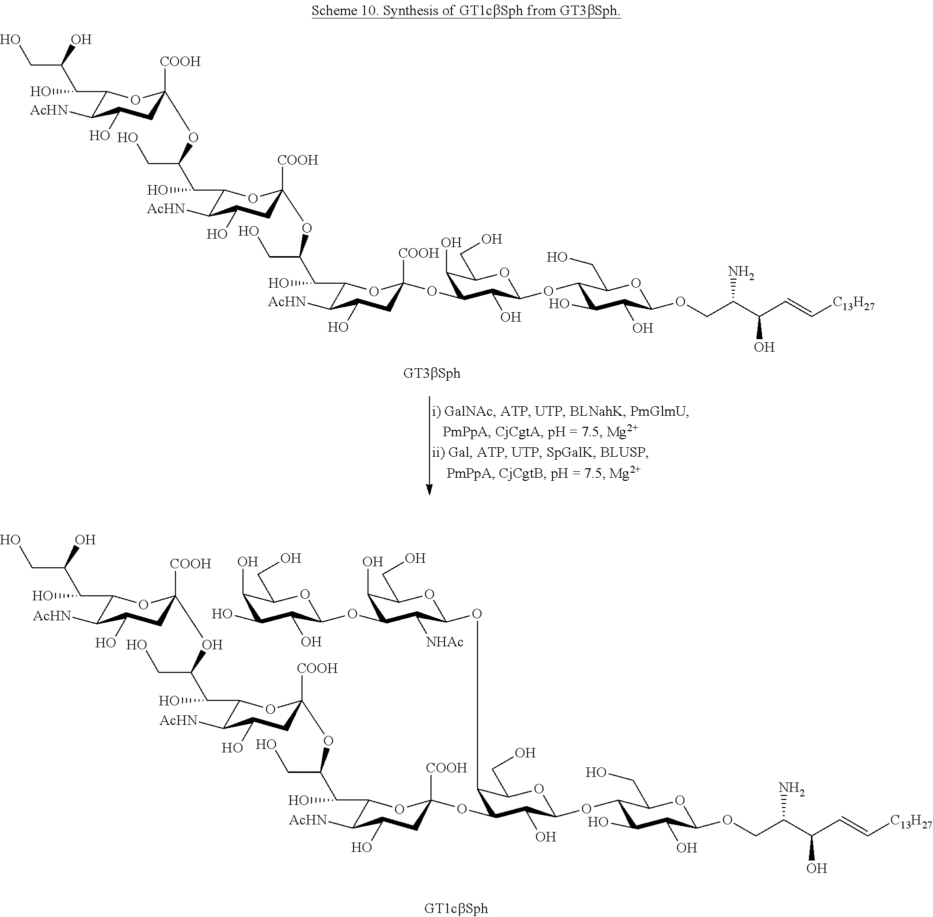

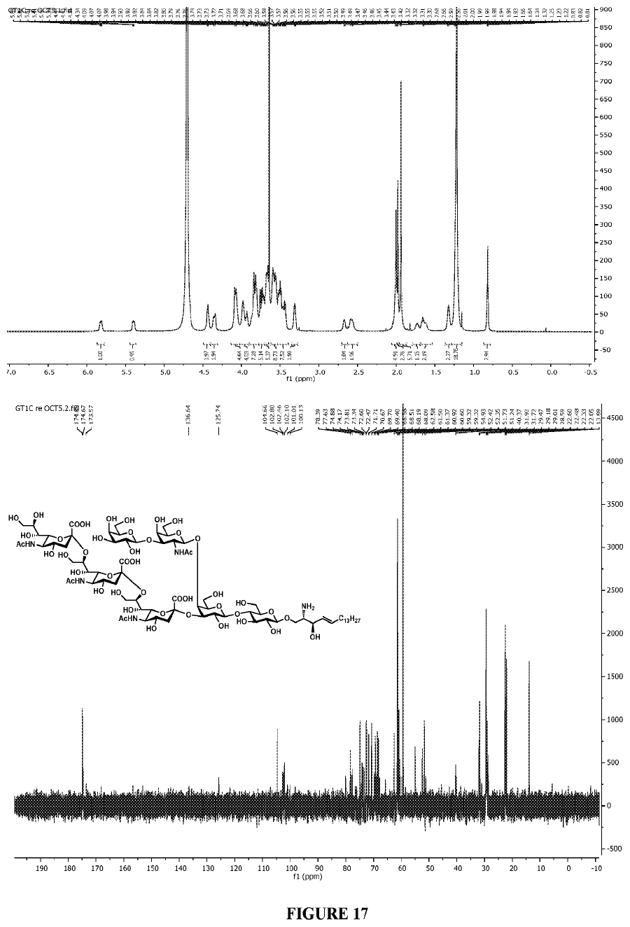

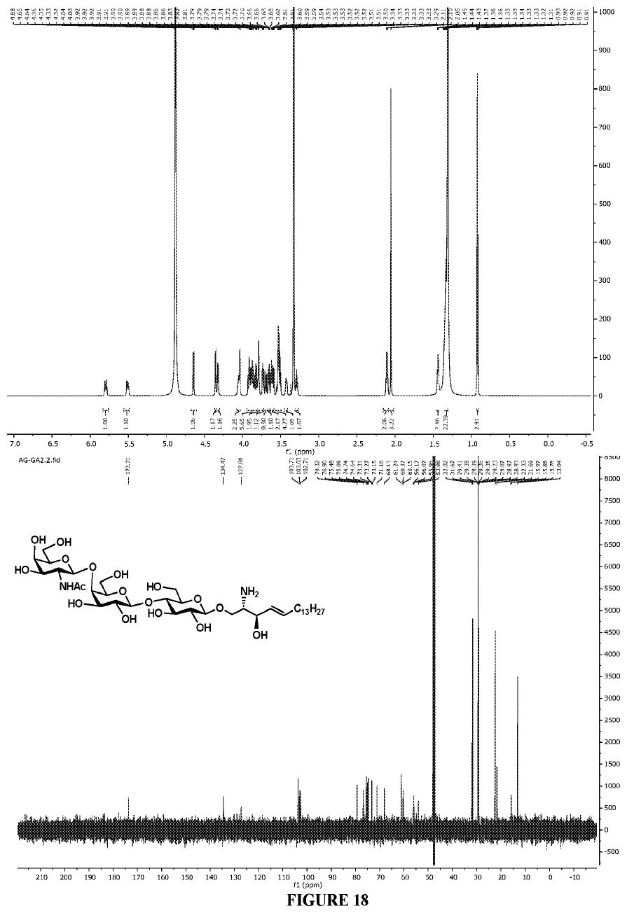

As shown in Scheme 10, GT1cβSph was synthesized from GT3βSph. A reaction mixture containing GT3βSph (250 mg, 0.16 mmol), GalNAc (73 mg, 0.33 mmol), ATP (182 mg, 0.33 mmol), UTP (174 mg, 0.33 mmol), sodium cholate (10 mM), BLNahK (4.0 mg), PmGlmU (4.0 mg), CjCgtA (7.5 mg), and PmPpA (2.0 mg) in 17 mL of Tris-HCl buffer (100 mM, pH 7.5) was incubated in an incubator shaker at 30° C. with agitation at 100 rpm. The product formation was monitored by FIRMS. After 48 hours, FIRMS indicated that the GT3βSph was almost consumed. Then the reaction mixture was incubated in a boiling water bath at 100° C. for 10 minutes and allowed to come to room temperature. In the same reaction container, galactose (60 mg, 0.33 mmol), ATP (182 mg, 0.33 mmol), UTP (174 g, 0.33 mmol), SpGalK (5 mg), BLUSP (5 mg), CjCgtB (8 mg), and PmPpA (2.0 mg) were added. The pH of the reaction mixture (22 mL) was adjusted to 7.5 by adding NaOH (4 M) and incubated in a shaker at 30° C. by agitating at 100 rpm. The product formation was monitored by HRMS. After 48 hours, HRMS indicated that the GT2βSph was almost consumed. Prechilled ethanol (22 mL) was added and the mixture was incubated at 4° C. for 30 minutes. The precipitates were removed by centrifugation and the supernatant was concentrated. The residue was purified using a 51 g ODS-SM column (50 M, 120 Å, Yamazen) on a CombiFlash® Rf 200i system. After loading the sample, the column was washed with water for 5 minutes and the product was eluted with 30% acetonitrile in water (v/v). The whole process took about 30 minutes. The fractions containing the product were collected, concentrated, and the residue was purified by silica gel column chromatography. A mixed solvent system of CH₂Cl₂:methanol=5:2 (by volume) was used to remove sodium cholate and CH₂Cl₂:methanol:water=5:4:1 (by volume) was used as an eluent to obtain the final pure GT1cβSph. The fractions containing the pure product were collected, concentrated, and lyophilized to obtain the final pure GT1cβSph as a white powder (295 mg, 95% yield). ¹H NMR (800 MHz, Deuterium Oxide) δ 5.87-5.77 (m, 1H), 5.39 (dd, J=15.1, 6.7 Hz, 1H), 4.44 (d, J=9.7 Hz, 2H), 4.35 (d, J=17.8 Hz, 2H), 4.13-4.02 (m, 5H), 4.02-3.90 (m, 4H), 3.90-3.78 (m, 7H), 3.78-3.70 (m, 5H), 3.70-3.65 (m, 5H), 3.62-3.53 (m, 9H), 3.53-3.40 (m, 8H), 3.35-3.27 (m, 2H), 2.67 (d, J=10.7 Hz, 1H), 2.58 (d, J=22.7 Hz, 2H), 2.01 (d, J=8.4 Hz, 5H), 1.98 (d, J=7.8 Hz, 3H), 1.94 (d, J=5.6 Hz, 6H), 1.73 (s, 1H), 1.65 (dd, J=28.1, 16.2 Hz, 2H), 1.33 (d, J=10.1 Hz, 2H), 1.22 (d, J=11.1 Hz, 19H), 0.82 (t, J=6.9 Hz, 3H). ¹³C NMR (200 MHz, Deuterium Oxide) δ 174.9, 174.7, 173.6, 136.6, 125.7, 104.7, 102.8, 102.5, 102.1, 101.0, 100.1, 78.4, 77.6, 74.9, 74.2, 73.8, 73.3, 72.6, 72.5, 71.7, 70.7, 69.7, 69.4, 69.0, 68.6, 68.5, 68.2, 68.1, 62.6, 61.5, 61.4, 60.9, 60.6, 59.3, 59.3, 54.9, 52.4, 52.4, 51.7, 51.2, 40.4, 31.9, 31.7, 29.5, 29.2, 29.0, 28.6, 22.6, 22.5, 22.3, 22.0, 13.9. FIG. 17 shows ¹H and ¹³C NMR spectra of GT1cβSph (d18:1).

Synthesis of GA2βSph from GM3βSph:

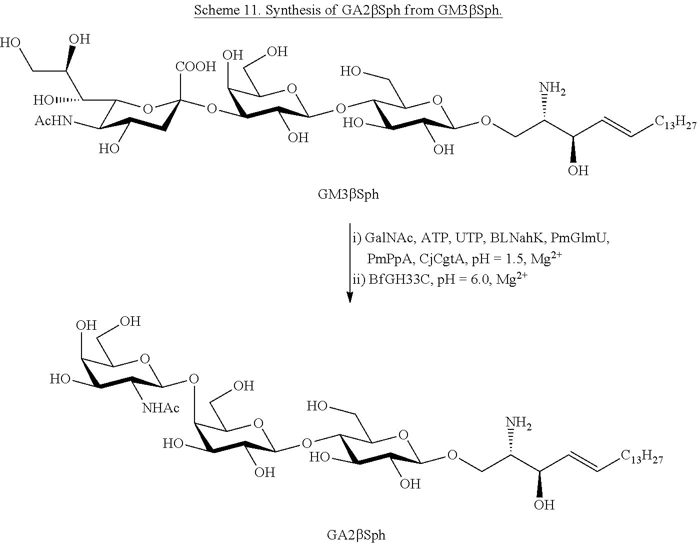

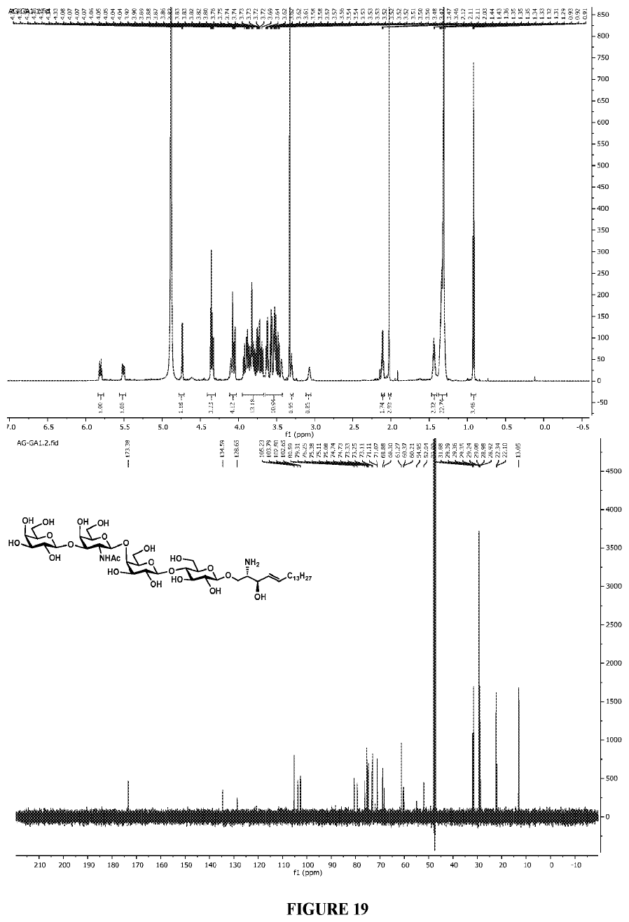

As shown in Scheme 11, GA2βSph was synthesized from GM3βSph. A reaction mixture containing GM3βSph (50 mg, 0.05 mmol), GalNAc (24 mg, 0.11 mmol), sodium cholate (10 mM), ATP (60.6 mg, 0.11 mmol), UTP (58.0 mg, 0.11 mmol), BLNahK (1.0 mg), PmGlmU (1.0 mg), CjCgtA (1.50 mg), and PmPpA (0.5 mg) in 5 mL of Tris-HCl buffer (100 mM, pH 7.5) was incubated in an incubator shaker at 30° C. with agitation at 100 rpm. The product formation was monitored by FIRMS. After 48 hours, the FIRMS indicated that the GM3 was almost consumed. Then the reaction mixture was incubated in a boiling water bath at 100° C. for 10 minutes and allowed to come to room temperature. In the same reaction container was added BfGH33C (1.0 mg). The pH of the reaction mixture was adjusted to 6 by adding HCl (2 N) and the reaction mixture was incubated in an incubator shaker at 30° C. with agitation at 100 rpm. After 24 hours, FIRMS indicated that the GM2βSph was almost consumed. Upon completion, the same volume (6 mL) of cold ethanol was added and the mixture was incubated at 4° C. for 30 minutes before it was centrifuged to remove precipitates. The supernatant was concentrated and the residue was purified using a 37 g ODS-SM column (50 μM, 120 Å, Yamazen) on a CombiFlash® Rf 200i system. After loading the sample, the column was washed with water for 5 minutes. The product was eluted with 60% acetonitrile in water (v/v). The whole process took about 25 minutes. The fractions containing the product were collected, concentrated, and the residue was purified by silica gel column chromatography. A mixed solvent system of CH₂Cl₂:methanol=5:2 (by volume) was used to remove sodium cholate and CH₂Cl₂:methanol:water=5:4:1 (by volume) was used as an eluent to obtain the final pure GA2βSph. The fractions containing the pure product were collected, concentrated, and lyophilized to obtain the final pure GA2βSph (33 mg, 73% yield) as a white powder. ¹H NMR (800 MHz,) δ 5.79 (dt, J=14.4, 6.8 Hz, 1H), 5.56-5.48 (m, 1H), 4.64 (d, J=8.4 Hz, 1H), 4.36 (dq, J=5.7, 2.7 Hz, 1H), 4.32 (d, J=7.8 Hz, 1H), 4.05 (dd, J=15.4, 4.5 Hz, 2H), 3.94-3.81 (m, 6H), 3.81-3.78 (m, 2H), 3.73 (dd, J=11.3, 4.5 Hz, 1H), 3.69 (dd, J=11.4, 6.4 Hz, 1H), 3.65 (dd, J=9.8, 3.1 Hz, 1H), 3.63-3.59 (m, 2H), 3.57-3.49 (m, 4H), 3.45-3.41 (m, 1H), 3.29 (t, J=8.1 Hz, 1H), 2.11 (q, J=7.2 Hz, 2H), 2.05 (s, 3H), 1.46-1.42 (m, 2H), 1.35-1.31 (m, 22H), 0.92 (t, J=7.1 Hz, 3H). ¹³C NMR (200 MHz, Methanol-d₄) δ 173.7, 134.5, 127.1, 103.7, 103.0, 102.7, 79.3, 76.9, 75.5, 75.1, 74.7, 74.6, 73.3, 73.3, 73.2, 71.2, 68.1, 61.2, 60.4, 60.2, 56.2, 56.1, 56.0, 54.0, 32.0, 31.7, 29.7, 29.6, 29.5, 29.4, 29.3, 29.2, 29.1, 29.0, 28.9, 22.3, 21.7, 16.0, 15.9, 15.8, 13.0. FIG. 18 shows ¹H and ¹³C NMR spectra of GA2βSph (d18:1).

Synthesis of GA1βSph from GM3βSph:

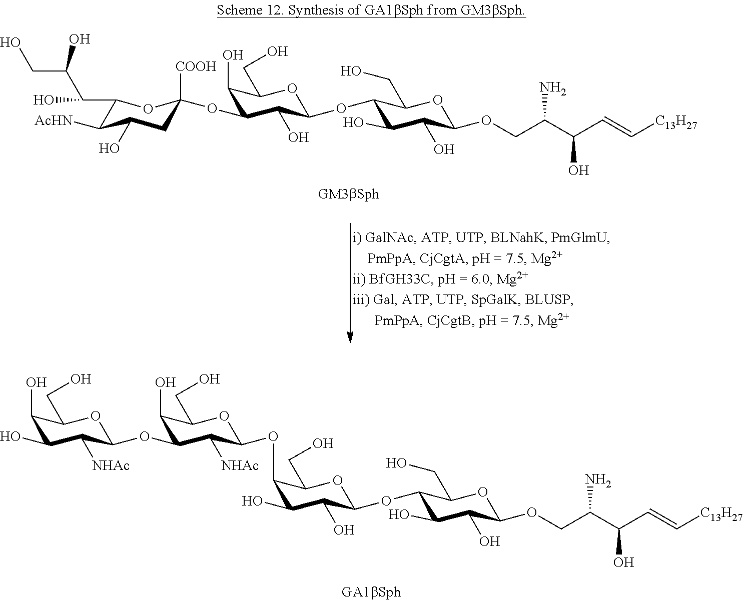

As shown in Scheme 12, GA1βSph was synthesized from GM3βSph. A reaction mixture containing GM3βSph (250 mg, 0.27 mmol), GalNAc (120 mg, 0.55 mmol), sodium cholate (10 mM), ATP (303.0 mg, 0.55 mmol), UTP (290.0 mg, 0.55 mmol), BLNahK (5.0 mg), PmGlmU (5.0 mg), CjCgtA (7.50 mg), and PmPpA (2.5 mg) in 25 mL of Tris-HCl buffer (100 mM, pH 7.5) was incubated in an incubator shaker at 30° C. with agitation at 100 rpm. The product formation was monitored by FIRMS. After 48 hours, the FIRMS indicated that the GM3 was almost consumed. Then the reaction mixture was incubated in a boiling water bath at 100° C. for 10 minutes and allowed to come to room temperature. In the same reaction container was added BfGH33C (5.0 mg). The pH of the reaction mixture (30 mL) was adjusted to 6 by adding HCl (2 N) and the reaction mixture was incubated in an incubator shaker at 30° C. with agitation at 100 rpm. After 24 hours, FIRMS indicated that the GM2βSph was almost consumed then the reaction mixture was incubated in a boiling water bath at 100° C. for 10 minutes and allowed to come to room temperature. In the same reaction container, galactose (99 mg, 0.55 mmol), ATP (303 mg, 0.55 mmol), UTP (290 mg, 0.55 mmol), SpGalK (7 mg), BLUSP (7 mg), CjCgtB (10 mg), and PmPpA (3.0 mg) were added. The pH of the reaction mixture (35 mL) was adjusted to 7.5 by adding NaOH (4 M) and incubated in a shaker at 30° C. by agitating at 100 rpm. The product formation was monitored by FIRMS. After 48 hours, FIRMS indicated that the GA2βSph was almost consumed. Upon completion, the same volume (35 mL) of cold ethanol was added and the mixture was incubated at 4° C. for 30 minutes before it was centrifuged to remove precipitates. The supernatant was concentrated and the residue was dissolved in 5 mL water. The sample was purified using a 51 g ODS-SM column (50 μM, 120 Å, Yamazen) on a CombiFlash® Rf 200i system. After loading the sample, the column was washed with water for 5 minutes. The product was eluted with 55% acetonitrile in water (v/v). The whole process took about 30 minutes. The fractions containing the product were collected, concentrated, and the residue was purified by silica gel column chromatography. A mixed solvent system of CH₂Cl₂:methanol=5:2 (by volume) was used to remove sodium cholate and CH₂Cl₂:methanol:water=5:4:1 (by volume) was used as an eluent to obtain the final pure GA1βSph. The fractions containing the pure product were collected, concentrated, and lyophilized to obtain the final pure GA1βSph (192 mg, 71% yield) as a white powder. ¹H NMR (800 MHz, Methanol-d₄) δ 5.81 (dt, J=14.4, 6.8 Hz, 1H), 5.51 (dd, J=15.4, 7.2 Hz, 1H), 4.73 (d, J=8.5 Hz, 1H), 4.41-4.30 (m, 3H), 4.11-4.03 (m, 4H), 3.95-3.67 (m, 13H), 3.65-3.42 (m, 11H), 3.32-3.28 (m, 1H), 3.07 (s, 1H), 2.12-2.09 (m, 2H), 2.03 (s, 3H), 1.44 (q, J=7.4 Hz, 2H), 1.38-1.27 (m, 23H), 0.92 (t, J=7.1 Hz, 3H). ¹³C NMR (200 MHz, Methanol-d₄) δ 173.4, 134.6, 128.7, 105.2, 103.8, 102.8, 102.6, 80.6, 79.3, 76.3, 75.4, 75.1, 75.1, 74.7, 74.7, 73.3, 73.2, 73.1, 71.1, 71.1, 68.9, 68.3, 61.3, 60.4, 60.2, 54.9, 52.0, 32.0, 31.7, 29.5, 29.4, 29.3, 29.2, 29.1, 29.0, 28.9, 22.3, 22.1, 13.0. FIG. 19 shows ¹H and ¹³C NMR spectra of GA1βSph (d18:1).

### Example 11: MBP-Δ15CjCgtA-His₆ PROSS Mutants

Adding an N-terminal MBP-fusion (MBP-Δ15CjCgtA-His₆) to the recombinant Δ15CjCgtA-His₆ (40 mg/L culture, precipitate during dialysis) improved its soluble expression level (85 mg/L culture) and stability. See Yu, H.; Zhang, L.; Yang, X.; Bai, Y.; Chen, X. Process engineering and glycosyltransferase improvement for short route chemoenzymatic total synthesis of GM1 gangliosides. Chem. Eur. J. 2023, 29, e202300005, incorporated herein by reference in its entirety. The resulting MBP-Δ15CjCgtA-His₆ was used for synthesis of GM2 and GM1 glycosphingosines. To further improve the soluble expression and stability of MBP-Δ15CjCgtA-His₆, Protein Repair One Stop Shop (PROSS), a computational approach that uses evolutionary information to suggest mutations, was used to design several mutants. Protein Repair One Stop Shop (PROSS) is described in Goldenzweig, A., Goldsmith, M., Hill, S. E., Gertman, O., Laurino, P., Ashani, Y., Dym, O., Unger, T., Albeck, S., Prilusky, J., Lieberman, R. L., Aharoni, A., Silman, I., Sussman, J. L., Tawfik, D. S. and Fleishman, S. J. Automated structure- and sequence-based design of proteins for high bacterial expression and stability. Mol Cell. 2016, 63, 337-346, and is incorporated herein by reference in its entirety.

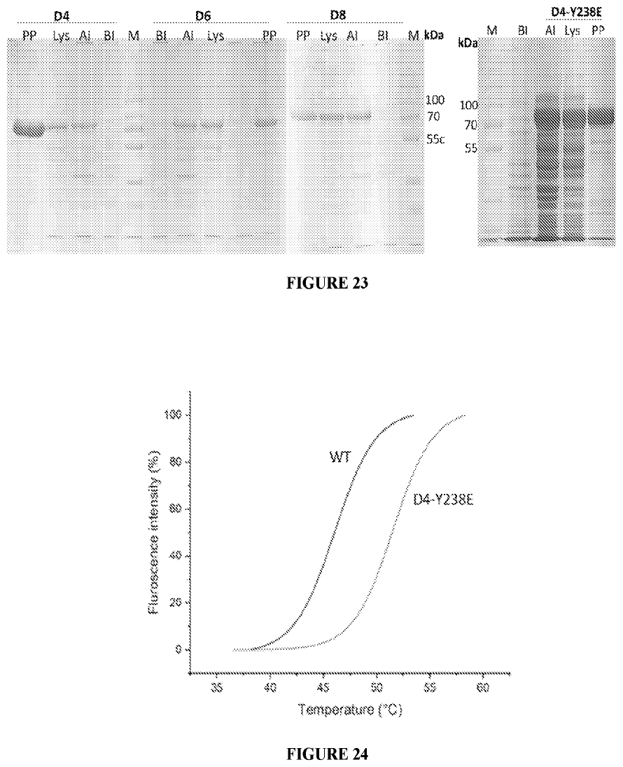

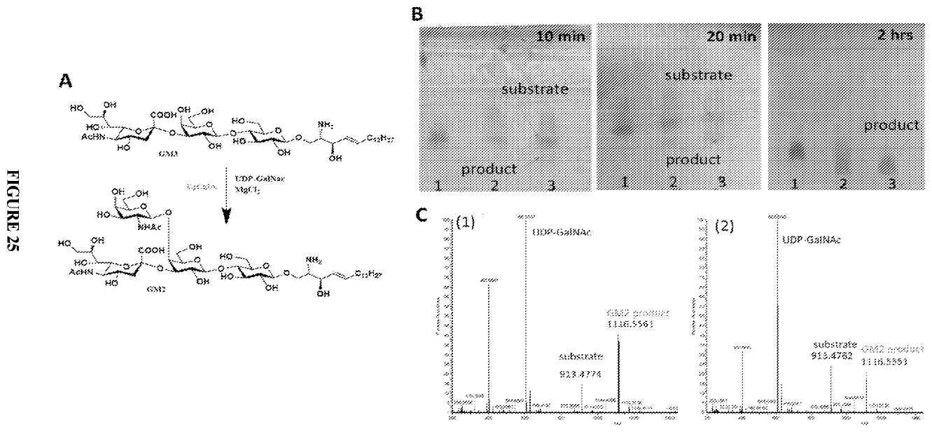

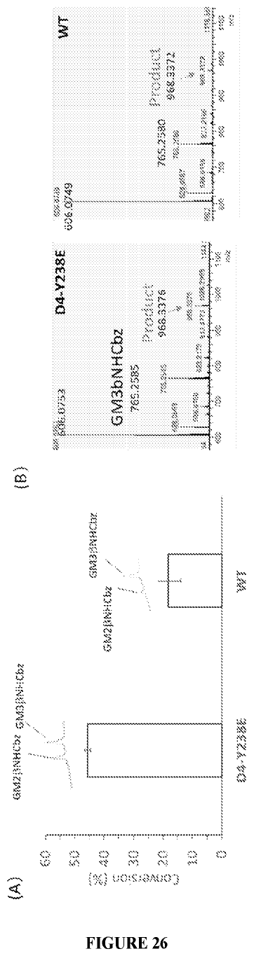

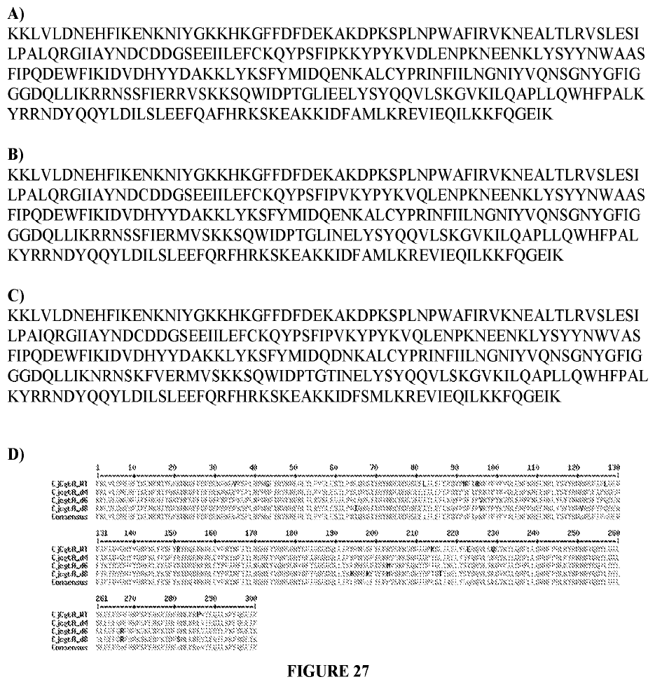

Three PROSS mutants D4, D6, and D8 (Table 4) were chosen to test the expression and analyzed by dodecyl sulfate-polyacrylamide gel electrophoresis (SDS-PAGE) assays (FIG. 23). Among the three mutants, D4 was shown to have the highest soluble expression 20 level (110 mg/L LB) in E. coli BL21(DE3) compared to the wild-type enzyme MBP-Δ15CjCgtA-His₆ and the other mutants, while the expression levels of D6 (75 mg/L LB) and D8 (50 mg/L LB) were lower than that of the wild-type enzyme. As the key catalytic residue E238 was mutated to Y in all three mutants, they were catalytically inactive. The Y238E mutant of D4 was constructed and the resulting D4-Y238E mutant was shown to be more stable (FIG. 24) and have a higher soluble expression level (102-117 mg/L LB) and improved activity (FIG. 25) than the wild-type enzyme MBP-Δ15CjCgtA-His₆ (Table 5). The pI values calculated using the online server, Protein Parameters (the amino acid pKa values from Bjellqvist et al were used) were 6.8 and 6.5, respectively, for the WT and its D4-Y238E mutant. Bjellqvist et al describes the amino acid pKa values that were used; Bjellqvist, B.; Hughes, G. J.; Pasquali, C.; Paquet, N.; Ravier, F.; Sanchez, J.-C.; Frutiger, S.; Hochstrasser, D., The focusing positions of polypeptides in immobilized pH gradients can be predicted from their amino acid sequences, Electrophoresis, 1993, 14, 1023-1031, which is incorporated herein by reference in its entirety. FIG. 26 shows activity comparison of MBP-Δ15CjCgtA-His₆ (WT) and its D4-Y238E mutant via UHPLC (Panel A) and HRMS (Panel B). Reaction conditions in FIG. 26 included GM3 βNHCbz 1 mM, UDP-GalNAc 1.5 mM, CjCgtA enzyme ~0.1 mg/mL, MgCl₂ (10 mM), Tris-HCl (100 mM, pH 7.4), 30° C., 10 min. The inset of FIG. 26 shows UHPLC peaks of the GM3P3NHCbz substrate and the GM2βNHCbz product. FIG. 27 shows the sequences of CjCgtA PROSS design D4 (Panel A, SEQ ID NO: 12), D6 (Panel B, SEQ ID NO: 13), and D8 (Panel C, SEQ ID NO: 14).

TABLE 4
The list of amino acid residues in MBP-Δ15CjCgtA-His₆ (WT) and in its
PROSS-designed mutants D4, D6, D8, and D4_Y238E.

| | | | | | D4- |
|---|---|---|---|---|---|
| Site | WT | D4 | D6 | D8 | Y238E |
| 50 | V | P | P | P | P |
| 58 | G | A | A | A | A |
| 80 | L | | | I | |
| 97 | L | I | I | I | I |
| 107 | N | S | S | S | S |
| 110 | A | P | P | P | P |
| 111 | K | | V | V | |
| 136 | A | | | V | |
| 142 | L | Q | Q | Q | Q |
| 166 | R | M | M | M | M |
| 209 | R | | | N | |
| 213 | S | | | K | |
| 215 | I | | | V | |
| 218 | R | | M | M | |
| 229 | K | T | T | T | T |
| 231 | L | | | T | |
| 238 | E | Y | Y | Y | E |
| 244 | Q | K | K | K | K |
| 282 | A | | R | R | |
| 296 | A | | | S | |
| 301 | P | E | E | E | E |

TABLE 5
Properties comparison of MBP-Δ15CjCgtA-His₆
(WT) and its D4-Y238E mutant.

| Properties | WT | D4-Y238E |
|---|---|---|
| Expression level | 80 mg/L LB | 102-117 mg/L LB |
| Tm (° C.) | 46.1 ± 0.7 | 51.5 ± 0.3 |
| pI | 6.8 | 6.5 |
| Protein extinction coefficient | 0.65 | 0.65 |

### Cloning

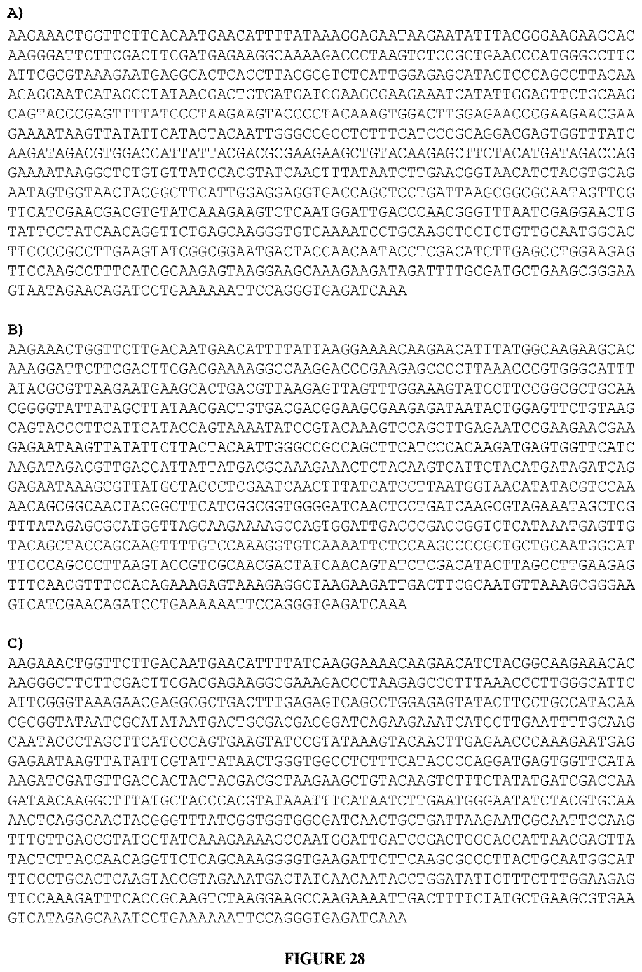

The codon optimized (for E. coli expression) gene fragments for Design D4 (D4) (FIG. 28, Panel A, SEQ ID NO: 15), Design D6 (D6) (FIG. 28, Panel B, SEQ ID NO: 16), Design D8 (D8) (FIG. 28, Panel C, SEQ ID NO: 17) were synthesized. The genes were cloned into pMAL-C2x vector. The primers used for cloning were: Forward, 5′-GACCGAATTCAAGAAACTGGTTCTTGACAATG-3′ (EcoRI restriction site sequence is underlined, SEQ ID NO: 18); Reverse, 5′-CAGCAAGCTTTTAGTGGTGGTGATGATGATG TTTGATCTCACCCTGG-3′ (HindIII restriction site sequence is underlined, SEQ ID NO: 19). The polymerase chain reaction (PCR) for amplifying the target gene was performed in a 50 μL reaction mixture containing the DNA fragment (10 ng), forward and reverse primer (0.2 μM each), 1×Phusion HF buffer, dNTP mixture (0.2 mM each), and 1 U (0.5 μL) of Phusion® High Fidelity DNA Polymerase. The reaction mixture was subjected to 30 cycles of amplification with an annealing temperature of 54° C. The resulting PCR product was purified and double digested with EcoRI and HindIII restriction enzymes. The digested and purified PCR product was ligated with the pMAL-c2X vector predigested with the same restriction enzymes and transformed into E. coli DH5α Z-competent cells. Selected clones were grown for plasmid minipreps and the gene sequences were confirmed by sequencing.

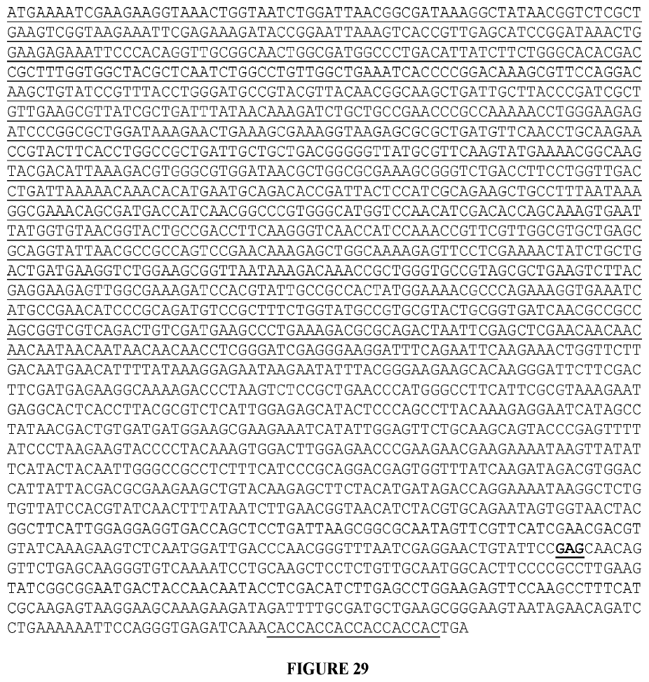

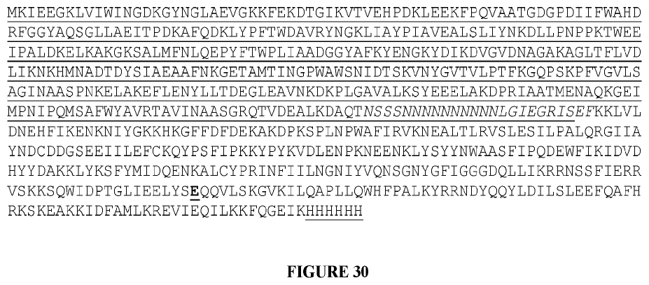

The D4-Y238E variant was constructed by site-directed mutation with a Q5 mutagenesis kit using the D4 gene in the pMAL-C2x plasmid as the template. The primers used were: Forward: 5′-ACTGTATTCCGAGCAACAGGTTC-3′ (SEQ ID NO: 20); Reverse, 5′-TCCTCGATTAAACCCGTTG-3′ (SEQ ID NO: 21). The annealing temperature was 59° C. E. coli DH5α Z-competent cells was used for the cloning. Selected clones were grown for plasmid minipreps and the gene sequences were confirmed by sequencing (FIG. 29, SEQ ID NO: 22). FIG. 30 shows the protein sequence of MBP-Δ15CjCgtA-His₆ D4-Y238E mutant (SEQ ID NO: 23). The sequences from the vector and the His₆ tag are underlined. The linker sequences are italicized. E238 is bolded and underlined.

### Enzyme Expression and Purification

Escherichia coli BL21(DE3) cells were transformed with the desired plasmid and plated on LB-Agar plate containing ampicillin (100 μg/mL). A single colony was inoculated into LB medium (10 g/L tryptone, 5 g/L yeast extract, 10 g/L NaCl) or 2YT medium (16 g/L tryptone, 10 g/L yeast extract, 5 g/L NaCl) supplemented with 100 μg/mL ampicillin. The cells were grown at 37° C. with shaking (220 rpm) overnight. About 20 mL of the overnight culture was inoculated in 1 L of LB or 2YT medium containing 100 μg/mL ampicillin, and incubated at 37° C. with shaking at 220 rpm. The cell culture was grown to an OD_{600 nm} of 0.6-0.8, at which point protein expression was induced with 100 μM isopropyl 3-D-1-thiogalactopyranoside (IPTG). The culture was incubated at 20° C. with shaking (220 rpm) for an additional 18-20 hours and cells were harvested by centrifugation for purification or storage at −20° C. until further use.

The proteins were purified using a nickel-nitrilotriacetic acid (Ni²⁺-NTA) affinity column. The cells harvested were re-suspended with a lysis buffer (100 mM Tris-HCl buffer, pH 8.0, 0.1% Triton X-100, 10% glycerol). The cells were homogenized by a homogenizer (EmulsiFlex-C3) and centrifuged at 8000 rpm for 60 minutes at 4° C. The supernatant was collected to obtain lysate which was fileted through a 0.45 μm filter, then loaded onto a pre-equilibrated Ni²⁺-NTA affinity column. The column was washed with 10-column volumes of a binding buffer (50 mM Tris-HCl buffer, pH 7.5, 25 mM imidazole, 0.5 M NaCl). The target protein was eluted using an elution buffer (50 mM Tris-HCl buffer, pH 7.5, 250 mM imidazole, 0.5 M NaCl).

### Thermal Shift Assays

To compare the thermostability of enzyme variants, protein thermal shift assays were performed using a fluorescence-based quantitative real-time PCR (qPCR)-based method. MBP-Δ15CjCgtA-His₆ (WT) or its D4-Y238E mutant was dialyzed against a dialysis buffer (Tris-HCl, 50 mM, pH 7.5 containing 250 mM of NaCl, and 10% of glycerol). WT and mutant enzymes were diluted to 0.75 mg/mL. Enzymes were tested in a MicroAmp™ Optical 96-Well Reaction Plate using Protein Thermal Shift™ Dye Kit. Wild type (WT) or mutant enzyme (17.5 μL) was mixed with 2.5 μL of 8×SYPRO Orange diluted dye. Data were acquired and analyzed in Protein Thermal Shift™ software. Tₘ was determined by system generated fluorescent intensity versus temperature plots. Each enzyme sample was tested in triplicates.

### Examples Summary

Two glycosyltransferases, CjCgtA and CjCgtB, have been engineered to increase their expression levels in E. coli and improve their stability. A multistep one-pot multienzyme (MSOPME) strategy has been successfully developed for enzymatic synthesis of GM1 sphingosine from lactosylsphingosine without the purification of intermediate glycosphingosines. The addition of a detergent (sodium cholate) has been found to drastically improve the glycosylation efficiency of glycosphingosines by CjCgtA and CjCgtB. The combined process and glycosyltransferase engineering strategies allow a quick access to GM1 (GM1a) gangliosides containing different sialic acid forms and different sphingosine structures. The OPME, MSOPME strategies and engineered CjCgtA and CjCgtB have also been used for synthesizing GM2, GD2, GD1b, GT2, GT1c, GA2, and GA1 glycosylsphingosines. They can be applied to synthesizing other glycosphingolipids, glycoconjugates, and glycans.

SEQUENCES

SEQ ID NO: 1:
MLKKIISLYKRYSISKKLVLDNEHFIKENKNIYGKKHKGFFDFDEKAKDVKSPLNPW
GFIRVKNEALTLRVSLESILPALQRGIIAYNDCDDGSEELILEFCKQYPNFIAKKYPYK
VDLENPKNEENKLYSYYNWAASFIPLDEWFIKIDVDHYYDAKKLYKSFYRIDQENK
ALCYPRINFIILNGNIYVQNSGNYGFIGGGDQLLIKRRNSSFIERRVSKKSQWIDPKGLI
EELYSEQQVLSQGVKILQAPLLQWHFPALKYRRNDYQQYLDILSLEEFQAFHRKSKE
AKKIDFAMLKRPVIEQILKKFQGEIK
SEQ ID NO: 2:
KKLVLDNEHFIKENKNIYGKKHKGFFDFDEKAKDVKSPLNPWGFIRVKNEALTLRVS
LESILPALQRGIIAYNDCDDGSEELILEFCKQYPNFIAKKYPYKVDLENPKNEENKLYS
YYNWAASFIPLDEWFIKIDVDHYYDAKKLYKSFYRIDQENKALCYPRINFIILNGNIY
VQNSGNYGFIGGGDQLLIKRRNSSFIERRVSKKSQWIDPKGLIEELYSEQQVLSQGVK
ILQAPLLQWHFPALKYRRNDYQQYLDILSLEEFQAFHRKSKEAKKIDFAMLKRPVIE
QILKKFQGEIK
SEQ ID NO: 3:
MFKISIILPTYNVEQYIARAIESCIKQTFKDIEIIVVDDCGNDNSINIAKEYSKKDKRIKII
HNEKNIGLLRARYEGVKVANSPYIMFLDPDDYLELNACEECIKILDEQDEVDLVFFN
AIVESNVISYKKFDFNSGFYSKKEFVKKIIAEKNLYWTMWGKLIRKKLYLEAFASLRL
EKDVKINMAEDVLLYYPMLSQAQKIAYMNCNLYHYVPNNNSICNTKNEVGVKNNI
QDLQLVLNYLRQNYILNKYCSVLYVLIKYLLYIQIYKIK
SEQ ID NO: 4:
ATGAAAATCGAAGAAGGTAAACTGGTAATCTGGATTAACGGCGATAAAGGCTAT
AACGGTCTCGCTGAAGTCGGTAAGAAATTCGAGAAAGATACCGGAATTAAAGTC
ACCGTTGAGCATCCGGATAAACTGGAAGAGAAATTCCCACAGGTTGCGGCAACT
GGCGATGGCCCTGACATTATCTTCTGGGCACACGACCGCTTTGGTGGCTACGCTC
AATCTGGCCTGTTGGCTGAAATCACCCCGGACAAAGCGTTCCAGGACAAGCTGT
ATCCGTTTACCTGGGATGCCGTACGTTACAACGGCAAGCTGATTGCTTACCCGAT
CGCTGTTGAAGCGTTATCGCTGATTTATAACAAAGATCTGCTGCCGAACCCGCCA
AAAACCTGGGAAGAGATCCCGGCGCTGGATAAAGAACTGAAAGCGAAAGGTAA
GAGCGCGCTGATGTTCAACCTGCAAGAACCGTACTTCACCTGGCCGCTGATTGCT
GCTGACGGGGGTTATGCGTTCAAGTATGAAAACGGCAAGTACGACATTAAAGAC
GTGGGCGTGGATAACGCTGGCGCGAAAGCGGGTCTGACCTTCCTGGTTGACCTG
ATTAAAAACAAACACATGAATGCAGACACCGATTACTCCATCGCAGAAGCTGCC
TTTAATAAAGGCGAAACAGCGATGACCATCAACGGCCCGTGGGCATGGTCCAAC
ATCGACACCAGCAAAGTGAATTATGGTGTAACGGTACTGCCGACCTTCAAGGGT
CAACCATCCAAACCGTTCGTTGGCGTGCTGAGCGCAGGTATTAACGCCGCCAGTC
CGAACAAAGAGCTGGCAAAAGAGTTCCTCGAAAACTATCTGCTGACTGATGAAG
GTCTGGAAGCGGTTAATAAAGACAAACCGCTGGGTGCCGTAGCGCTGAAGTCTT
ACGAGGAAGAGTTGGCGAAAGATCCACGTATTGCCGCCACTATGGAAAACGCCC
AGAAAGGTGAAATCATGCCGAACATCCCGCAGATGTCCGCTTTCTGGTATGCCGT
GCGTACTGCGGTGATCAACGCCGCCAGCGGTCGTCAGACTGTCGATGAAGCCCT
GAAAGACGCGCAGACTAATTCGAGCTCGAACAACAACAACAATAACAATAACA
ACAACCTCGGGATCGAGGGAAGGATTTCAGAATTCAAGAAACTGGTGCTGGACA
ACGAGCACTTTATTAAGGAAAACAAAAACATCTACGGCAAGAAGCACAAGGGTT
TCTTCGATTTCGATGAGAAGGCAAAGGATGTGAAGAGCCCGCTGAATCCGTGGG
GTTTTATTCGTGTTAAGAATGAAGCACTGACCCTGCGTGTTAGCCTGGAAAGCAT
TCTGCCAGCCCTGCAACGTGGCATTATTGCGTATAATGACTGTGATGATGGCAGC
GAAGAACTGATCCTGGAGTTTTGCAAGCAGTACCCGAACTTTATCGCCAAAAAG
TATCCGTACAAGGTGGACCTGGAGAACCCGAAAAACGAAGAGAACAAACTGTAC
AGCTACTATAATTGGGCGGCAAGTTTTATTCCGCTGGATGAGTGGTTCATCAAGA
TCGACGTGGACCATTACTATGACGCGAAAAAGCTGTATAAGAGCTTCTACCGCAT
TGACCAGGAAAATAAGGCGCTGTGTTACCCACGCATCAACTTCATCATCCTGAAC
GGCAACATTTACGTGCAAAATAGCGGTAACTATGGTTTCATTGGTGGCGGTGACC
AACTGCTGATCAAACGTCGTAATAGCAGCTTCATCGAGCGTCGCGTTAGCAAAA
AAAGCCAGTGGATCGACCCGAAGGGTCTGATTGAGGAACTGTATAGCGAACAGC
AGGTGCTGAGCCAAGGCGTGAAAATTCTGCAGGCGCCACTGCTGCAATGGCACT
TCCCGGCACTGAAATATCGCCGCAATGACTACCAGCAATACCTGGATATTCTGAG
CCTGGAGGAATTTCAGGCCTTCCACCGTAAGAGCAAGGAAGCAAAGAAAATCGA
CTTTGCCATGCTGAAGCGCCCAGTTATTGAGCAGATTCTGAAGAAGTTTCAGGGT
GAGATCAAACACCACCACCACCACCACTAA
SEQ ID NO: 5:
MKIEEGKLVIWINGDKGYNGLAEVGKKFEKDTGIKVTVEHPDKLEEKFPQVAATGD
GPDIIFWAHDRFGGYAQSGLLAEITPDKAFQDKLYPFTWDAVRYNGKLIAYPIAVEA
LSLIYNKDLLPNPPKTWEEIPALDKELKAKGKSALMFNLQEPYFTWPLIAADGGYAF
KYENGKYDIKDVGVDNAGAKAGLTFLVDLIKNKHMNADTDYSIAEAAFNKGETAM
TINGPWAWSNIDTSKVNYGVTVLPTFKGQPSKPFVGVLSAGINAASPNKELAKEFLE
NYLLTDEGLEAVNKDKPLGAVALKSYEEELAKDPRIAATMENAQKGEIMPNIPQMS
AFWYAVRTAVINAASGRQTVDEALKDAQTNSSSNNNNNNNNNNLGIEGRISEFKKL
VLDNEHFIKENKNIYGKKHKGFFDFDEKAKDVKSPLNPWGFIRVKNEALTLRVSLESI
LPALQRGIIAYNDCDDGSEELILEFCKQYPNFIAKKYPYKVDLENPKNEENKLYSYYN
WAASFIPLDEWFIKIDVDHYYDAKKLYKSFYRIDQENKALCYPRINFIILNGNIYVQN
SGNYGFIGGGDQLLIKRRNSSFIERRVSKKSQWIDPKGLIEELYSEQQVLSQGVKILQA
PLLQWHFPALKYRRNDYQQYLDILSLEEFQAFHRKSKEAKKIDFAMLKRPVIEQILK
KFQGEIKHHHHHH
SEQ ID NO: 6:
ATGAAAATCGAAGAAGGTAAACTGGTAATCTGGATTAACGGCGATAAAGGCTAT
AACGGTCTCGCTGAAGTCGGTAAGAAATTCGAGAAAGATACCGGAATTAAAGTC
ACCGTTGAGCATCCGGATAAACTGGAAGAGAAATTCCCACAGGTTGCGGCAACT
GGCGATGGCCCTGACATTATCTTCTGGGCACACGACCGCTTTGGTGGCTACGCTC
AATCTGGCCTGTTGGCTGAAATCACCCCGGACAAAGCGTTCCAGGACAAGCTGT
ATCCGTTTACCTGGGATGCCGTACGTTACAACGGCAAGCTGATTGCTTACCCGAT
CGCTGTTGAAGCGTTATCGCTGATTTATAACAAAGATCTGCTGCCGAACCCGCCA
AAAACCTGGGAAGAGATCCCGGCGCTGGATAAAGAACTGAAAGCGAAAGGTAA
GAGCGCGCTGATGTTCAACCTGCAAGAACCGTACTTCACCTGGCCGCTGATTGCT
GCTGACGGGGGTTATGCGTTCAAGTATGAAAACGGCAAGTACGACATTAAAGAC
GTGGGCGTGGATAACGCTGGCGCGAAAGCGGGTCTGACCTTCCTGGTTGACCTG
ATTAAAAACAAACACATGAATGCAGACACCGATTACTCCATCGCAGAAGCTGCC
TTTAATAAAGGCGAAACAGCGATGACCATCAACGGCCCGTGGGCATGGTCCAAC
ATCGACACCAGCAAAGTGAATTATGGTGTAACGGTACTGCCGACCTTCAAGGGT
CAACCATCCAAACCGTTCGTTGGCGTGCTGAGCGCAGGTATTAACGCCGCCAGTC
CGAACAAAGAGCTGGCAAAAGAGTTCCTCGAAAACTATCTGCTGACTGATGAAG
GTCTGGAAGCGGTTAATAAAGACAAACCGCTGGGTGCCGTAGCGCTGAAGTCTT
ACGAGGAAGAGTTGGCGAAAGATCCACGTATTGCCGCCACTATGGAAAACGCCC
AGAAAGGTGAAATCATGCCGAACATCCCGCAGATGTCCGCTTTCTGGTATGCCGT
GCGTACTGCGGTGATCAACGCCGCCAGCGGTCGTCAGACTGTCGATGAAGCCCT
GAAAGACGCGCAGACTAATTCGAGCTCGAACAACAACAACAATAACAATAACA
ACAACCTCGGGATCGAGGGAAGGATTTCAGAATTCTTCAAAATTTCTATCATCCT
GCCGACCTATAACGTGGAACAGTATATCGCTCGCGCAATCGAGAGCTGCATCAA
ACAGACCTTCAAGGACATCGAGATTATCGTGGTGGACGACTGCGGCAACGATAA
CTCCATCAACATTGCTAAGGAATACAGCAAGAAGGACAAGCGTATCAAAATCAT
TCACAACGAAAAGAACATCGGCCTGCTGCGTGCACGTTACGAAGGTGTTAAAGT
GGCGAACAGCCCGTACATCATGTTCCTGGACCCGGACGATTATCTGGAGCTGAA
CGCGTGCGAAGAGTGCATTAAGATTCTGGACGAGCAGGACGAAGTGGACCTGGT
GTTCTTCAACGCTATTGTGGAGTCCAACGTGATCAGCTACAAGAAATTCGATTTT
AACTCCGGCTTCTACTCCAAAAAGGAGTTCGTGAAAAAGATCATCGCAGAGAAG
AACCTGTACTGGACCATGTGGGGCAAACTGATTCGCAAGAAACTGTACCTGGAG
GCGTTCGCTTCCCTGCGTCTGGAAAAGGATGTTAAGATCAACATGGCGGAAGAC
GTTCTGCTGTACTACCCGATGCTGTCTCAGGCACAGAAAATTGCGTACATGAACT
GCAACCTGTACCACTATGTGCCGAACAACAACTCTATTTGCAACACCAAAAACG
AGGTGGGCGTTAAAAACAACATTCAGGACCTGCAGCTGGTGCTGAACTACCTGC
GCCAGAACTACATCCTGAACAAGTACTGCAGCGTTCTGTACGTGCTGATTAAGTA
CCTGCTGTATATTCAGATCTACAAAATTAAGCATCATCATCACCACCACTAA
SEQ ID NO: 7:
MKIEEGKLVIWINGDKGYNGLAEVGKKFEKDTGIKVTVEHPDKLEEKFPQVAATGD
GPDIIFWAHDRFGGYAQSGLLAEITPDKAFQDKLYPFTWDAVRYNGKLIAYPIAVEA
LSLIYNKDLLPNPPKTWEEIPALDKELKAKGKSALMENLQEPYFTWPLIAADGGYAF
KYENGKYDIKDVGVDNAGAKAGLTFLVDLIKNKHMNADTDYSIAEAAFNKGETAM
TINGPWAWSNIDTSKVNYGVTVLPTFKGQPSKPFVGVLSAGINAASPNKELAKEFLE
NYLLTDEGLEAVNKDKPLGAVALKSYEEELAKDPRIAATMENAQKGEIMPNIPQMS
AFWYAVRTAVINAASGRQTVDEALKDAQTNSSSNNNNNNNNNNLGIEGRISEFFKIS
IILPTYNVEQYIARAIESCIKQTFKDIEIIVVDDCGNDNSINIAKEYSKKDKRIKIIHNEK
NIGLLRARYEGVKVANSPYIMFLDPDDYLELNACEECIKILDEQDEVDLVFFNAIVES
NVISYKKFDFNSGFYSKKEFVKKIIAEKNLYWTMWGKLIRKKLYLEAFASLRLEKDV
KINMAEDVLLYYPMLSQAQKIAYMNCNLYHYVPNNNSICNTKNEVGVKNNIQDLQ
LVLNYLRQNYILNKYCSVLYVLIKYLLYIQIYKIKHHHHHH
SEQ ID NO: 8:
5′-GACCGAATTC GTGCTGGACAACGAGCAC-3′
SEQ ID NO: 9:
5′-CAGCAAGCTTTCAGTGGTGGTGGTGGTG-3′
SEQ ID NO: 10:
5′-GACCGAATTCTTCAAAATTTCTATCATCCTGCCG 3′
SEQ ID NO: 11:
5′-
CAGCAAGCTTTTAGTGGTGGTGATGATGATGCTTAATTTTGTAGATCTGAATATAC-3′
SEQ ID NO: 12:
KKLVLDNEHFIKENKNIYGKKHKGFFDFDEKAKDPKSPLNPWAFIRVKNEALTLRVS
LESILPALQRGIIAYNDCDDGSEEIILEFCKQYPSFIPKKYPYKVDLENPKNEENKLYSY
YNWAASFIPQDEWFIKIDVDHYYDAKKLYKSFYMIDQENKALCYPRINFIILNGNIYV
QNSGNYGFIGGGDQLLIKRRNSSFIERRVSKKSQWIDPTGLIEELYSYQQVLSKGVKIL
QAPLLQWHFPALKYRRNDYQQYLDILSLEEFQAFHRKSKEAKKIDFAMLKREVIEQI
LKKFQGEIK
SEQ ID NO: 13:
KKLVLDNEHFIKENKNIYGKKHKGFFDFDEKAKDPKSPLNPWAFIRVKNEALTLRVS
LESILPALQRGIIAYNDCDDGSEEIILEFCKQYPSFIPVKYPYKVQLENPKNEENKLYSY
YNWAASFIPQDEWFIKIDVDHYYDAKKLYKSFYMIDQENKALCYPRINFIILNGNIYV
QNSGNYGFIGGGDQLLIKRRNSSFIERMVSKKSQWIDPTGLINELYSYQQVLSKGVKI
LQAPLLQWHFPALKYRRNDYQQYLDILSLEEFQRFHRKSKEAKKIDFAMLKREVIEQ
ILKKFQGEIK
SEQ ID NO: 14:
KKLVLDNEHFIKENKNIYGKKHKGFFDFDEKAKDPKSPLNPWAFIRVKNEALTLRVS
LESILPAIQRGIIAYNDCDDGSEEIILEFCKQYPSFIPVKYPYKVQLENPKNEENKLYSY
YNWVASFIPQDEWFIKIDVDHYYDAKKLYKSFYMIDQDNKALCYPRINFIILNGNIYV
QNSGNYGFIGGGDQLLIKNRNSKFVERMVSKKSQWIDPTGTINELYSYQQVLSKGVK
ILQAPLLQWHFPALKYRRNDYQQYLDILSLEEFQRFHRKSKEAKKIDFSMLKREVIEQ
ILKKFQGEIK
SEQ ID NO: 15:
AAGAAACTGGTTCTTGACAATGAACATTTTATAAAGGAGAATAAGAATATTTAC
GGGAAGAAGCACAAGGGATTCTTCGACTTCGATGAGAAGGCAAAAGACCCTAAG
TCTCCGCTGAACCCATGGGCCTTCATTCGCGTAAAGAATGAGGCACTCACCTTAC
GCGTCTCATTGGAGAGCATACTCCCAGCCTTACAAAGAGGAATCATAGCCTATA
ACGACTGTGATGATGGAAGCGAAGAAATCATATTGGAGTTCTGCAAGCAGTACC
CGAGTTTTATCCCTAAGAAGTACCCCTACAAAGTGGACTTGGAGAACCCGAAGA
ACGAAGAAAATAAGTTATATTCATACTACAATTGGGCCGCCTCTTTCATCCCGCA
GGACGAGTGGTTTATCAAGATAGACGTGGACCATTATTACGACGCGAAGAAGCT
GTACAAGAGCTTCTACATGATAGACCAGGAAAATAAGGCTCTGTGTTATCCACGT
ATCAACTTTATAATCTTGAACGGTAACATCTACGTGCAGAATAGTGGTAACTACG
GCTTCATTGGAGGAGGTGACCAGCTCCTGATTAAGCGGCGCAATAGTTCGTTCAT
CGAACGACGTGTATCAAAGAAGTCTCAATGGATTGACCCAACGGGTTTAATCGA
GGAACTGTATTCCTATCAACAGGTTCTGAGCAAGGGTGTCAAAATCCTGCAAGCT
CCTCTGTTGCAATGGCACTTCCCCGCCTTGAAGTATCGGCGGAATGACTACCAAC
AATACCTCGACATCTTGAGCCTGGAAGAGTTCCAAGCCTTTCATCGCAAGAGTAA
GGAAGCAAAGAAGATAGATTTTGCGATGCTGAAGCGGGAAGTAATAGAACAGAT
CCTGAAAAAATTCCAGGGTGAGATCAAA
SEQ ID NO: 16
AAGAAACTGGTTCTTGACAATGAACATTTTATTAAGGAAAACAAGAACATTTATG
GCAAGAAGCACAAAGGATTCTTCGACTTCGACGAAAAGGCCAAGGACCCGAAG
AGCCCCTTAAACCCGTGGGCATTTATACGCGTTAAGAATGAAGCACTGACGTTAA
GAGTTAGTTTGGAAAGTATCCTTCCGGCGCTGCAACGGGGTATTATAGCTTATAA
CGACTGTGACGACGGAAGCGAAGAGATAATACTGGAGTTCTGTAAGCAGTACCC
TTCATTCATACCAGTAAAATATCCGTACAAAGTCCAGCTTGAGAATCCGAAGAAC
GAAGAGAATAAGTTATATTCTTACTACAATTGGGCCGCCAGCTTCATCCCACAAG
ATGAGTGGTTCATCAAGATAGACGTTGACCATTATTATGACGCAAAGAAACTCTA
CAAGTCATTCTACATGATAGATCAGGAGAATAAAGCGTTATGCTACCCTCGAATC
AACTTTATCATCCTTAATGGTAACATATACGTCCAAAACAGCGGCAACTACGGCT
TCATCGGCGGTGGGGATCAACTCCTGATCAAGCGTAGAAATAGCTCGTTTATAGA
GCGCATGGTTAGCAAGAAAAGCCAGTGGATTGACCCGACCGGTCTCATAAATGA
GTTGTACAGCTACCAGCAAGTTTTGTCCAAAGGTGTCAAAATTCTCCAAGCCCCG
CTGCTGCAATGGCATTTCCCAGCCCTTAAGTACCGTCGCAACGACTATCAACAGT
ATCTCGACATACTTAGCCTTGAAGAGTTTCAACGTTTCCACAGAAAGAGTAAAGA
GGCTAAGAAGATTGACTTCGCAATGTTAAAGCGGGAAGTCATCGAACAGATCCT
GAAAAAATTCCAGGGTGAGATCAAA
SEQ ID NO: 17
AAGAAACTGGTTCTTGACAATGAACATTTTATCAAGGAAAACAAGAACATCTAC
GGCAAGAAACACAAGGGCTTCTTCGACTTCGACGAGAAGGCGAAAGACCCTAAG
AGCCCTTTAAACCCTTGGGCATTCATTCGGGTAAAGAACGAGGCGCTGACTTTGA
GAGTCAGCCTGGAGAGTATACTTCCTGCCATACAACGCGGTATAATCGCATATAA
TGACTGCGACGACGGATCAGAAGAAATCATCCTTGAATTTTGCAAGCAATACCCT
AGCTTCATCCCAGTGAAGTATCCGTATAAAGTACAACTTGAGAACCCAAAGAAT
GAGGAGAATAAGTTATATTCGTATTATAACTGGGTGGCCTCTTTCATACCCCAGG
ATGAGTGGTTCATAAAGATCGATGTTGACCACTACTACGACGCTAAGAAGCTGT
ACAAGTCTTTCTATATGATCGACCAAGATAACAAGGCTTTATGCTACCCACGTAT
AAATTTCATAATCTTGAATGGGAATATCTACGTGCAAAACTCAGGCAACTACGG
GTTTATCGGTGGTGGCGATCAACTGCTGATTAAGAATCGCAATTCCAAGTTTGTT
GAGCGTATGGTATCAAAGAAAAGCCAATGGATTGATCCGACTGGGACCATTAAC
GAGTTATACTCTTACCAACAGGTTCTCAGCAAAGGGGTGAAGATTCTTCAAGCGC
CCTTACTGCAATGGCATTTCCCTGCACTCAAGTACCGTAGAAATGACTATCAACA
ATACCTGGATATTCTTTCTTTGGAAGAGTTCCAAAGATTTCACCGCAAGTCTAAG
GAAGCCAAGAAAATTGACTTTTCTATGCTGAAGCGTGAAGTCATAGAGCAAATC
CTGAAAAAATTCCAGGGTGAGATCAAA
SEQ ID NO: 18
5′-GACCGAATTCAAGAAACTGGTTCTTGACAATG-3′
SEQ ID NO: 19
5′-CAGCAAGCTTTTAGTGGTGGTGATGATGATG TTTGATCTCACCCTGG-3′
SEQ ID NO: 20
5′-ACTGTATTCCGAGCAACAGGTTC-3′
SEQ ID NO: 21
5′-TCCTCGATTAAACCCGTTG-3′
SEQ ID NO: 22
ATGAAAATCGAAGAAGGTAAACTGGTAATCTGGATTAACGGCGATAAAGGCTAT
AACGGTCTCGCTGAAGTCGGTAAGAAATTCGAGAAAGATACCGGAATTAAAGTC
ACCGTTGAGCATCCGGATAAACTGGAAGAGAAATTCCCACAGGTTGCGGCAACT
GGCGATGGCCCTGACATTATCTTCTGGGCACACGACCGCTTTGGTGGCTACGCTC
AATCTGGCCTGTTGGCTGAAATCACCCCGGACAAAGCGTTCCAGGACAAGCTGT
ATCCGTTTACCTGGGATGCCGTACGTTACAACGGCAAGCTGATTGCTTACCCGAT
CGCTGTTGAAGCGTTATCGCTGATTTATAACAAAGATCTGCTGCCGAACCCGCCA
AAAACCTGGGAAGAGATCCCGGCGCTGGATAAAGAACTGAAAGCGAAAGGTAA
GAGCGCGCTGATGTTCAACCTGCAAGAACCGTACTTCACCTGGCCGCTGATTGCT
GCTGACGGGGGTTATGCGTTCAAGTATGAAAACGGCAAGTACGACATTAAAGAC
GTGGGCGTGGATAACGCTGGCGCGAAAGCGGGTCTGACCTTCCTGGTTGACCTG
ATTAAAAACAAACACATGAATGCAGACACCGATTACTCCATCGCAGAAGCTGCC
TTTAATAAAGGCGAAACAGCGATGACCATCAACGGCCCGTGGGCATGGTCCAAC
ATCGACACCAGCAAAGTGAATTATGGTGTAACGGTACTGCCGACCTTCAAGGGT
CAACCATCCAAACCGTTCGTTGGCGTGCTGAGCGCAGGTATTAACGCCGCCAGTC
CGAACAAAGAGCTGGCAAAAGAGTTCCTCGAAAACTATCTGCTGACTGATGAAG
GTCTGGAAGCGGTTAATAAAGACAAACCGCTGGGTGCCGTAGCGCTGAAGTCTT
ACGAGGAAGAGTTGGCGAAAGATCCACGTATTGCCGCCACTATGGAAAACGCCC
AGAAAGGTGAAATCATGCCGAACATCCCGCAGATGTCCGCTTTCTGGTATGCCGT
GCGTACTGCGGTGATCAACGCCGCCAGCGGTCGTCAGACTGTCGATGAAGCCCT
GAAAGACGCGCAGACTAATTCGAGCTCGAACAACAACAACAATAACAATAACA
ACAACCTCGGGATCGAGGGAAGGATTTCAGAATTCAAGAAACTGGTTCTTGACA
ATGAACATTTTATAAAGGAGAATAAGAATATTTACGGGAAGAAGCACAAGGGAT
TCTTCGACTTCGATGAGAAGGCAAAAGACCCTAAGTCTCCGCTGAACCCATGGG
CCTTCATTCGCGTAAAGAATGAGGCACTCACCTTACGCGTCTCATTGGAGAGCAT
ACTCCCAGCCTTACAAAGAGGAATCATAGCCTATAACGACTGTGATGATGGAAG
CGAAGAAATCATATTGGAGTTCTGCAAGCAGTACCCGAGTTTTATCCCTAAGAAG
TACCCCTACAAAGTGGACTTGGAGAACCCGAAGAACGAAGAAAATAAGTTATAT
TCATACTACAATTGGGCCGCCTCTTTCATCCCGCAGGACGAGTGGTTTATCAAGA
TAGACGTGGACCATTATTACGACGCGAAGAAGCTGTACAAGAGCTTCTACATGA
TAGACCAGGAAAATAAGGCTCTGTGTTATCCACGTATCAACTTTATAATCTTGAA
CGGTAACATCTACGTGCAGAATAGTGGTAACTACGGCTTCATTGGAGGAGGTGA
CCAGCTCCTGATTAAGCGGCGCAATAGTTCGTTCATCGAACGACGTGTATCAAAG
AAGTCTCAATGGATTGACCCAACGGGTTTAATCGAGGAACTGTATTCCGAGCAA
CAGGTTCTGAGCAAGGGTGTCAAAATCCTGCAAGCTCCTCTGTTGCAATGGCACT
TCCCCGCCTTGAAGTATCGGCGGAATGACTACCAACAATACCTCGACATCTTGAG
CCTGGAAGAGTTCCAAGCCTTTCATCGCAAGAGTAAGGAAGCAAAGAAGATAGA
TTTTGCGATGCTGAAGCGGGAAGTAATAGAACAGATCCTGAAAAAATTCCAGGG
TGAGATCAAACACCACCACCACCACCACTGA
SEQ ID NO: 23
MKIEEGKLVIWINGDKGYNGLAEVGKKFEKDTGIKVTVEHPDKLEEKFPQVAATGD
GPDIIFWAHDRFGGYAQSGLLAEITPDKAFQDKLYPFTWDAVRYNGKLIAYPIAVEA
LSLIYNKDLLPNPPKTWEEIPALDKELKAKGKSALMFNLQEPYFTWPLIAADGGYAF
KYENGKYDIKDVGVDNAGAKAGLTFLVDLIKNKHMNADTDYSIAEAAFNKGETAM
TINGPWAWSNIDTSKVNYGVTVLPTFKGQPSKPFVGVLSAGINAASPNKELAKEFLE
NYLLTDEGLEAVNKDKPLGAVALKSYEEELAKDPRIAATMENAQKGEIMPNIPQMS
AFWYAVRTAVINAASGRQTVDEALKDAQTNSSSNNNNNNNNNNLGIEGRISEFKKL
VLDNEHFIKENKNIYGKKHKGFFDFDEKAKDPKSPLNPWAFIRVKNEALTLRVSLESI
LPALQRGIIAYNDCDDGSEEIILEFCKQYPSFIPKKYPYKVDLENPKNEENKLYSYYN
WAASFIPQDEWFIKIDVDHYYDAKKLYKSFYMIDQENKALCYPRINFIILNGNIYVQN
SGNYGFIGGGDQLLIKRRNSSFIERRVSKKSQWIDPTGLIEELYSEQQVLSKGVKILQA
PLLQWHFPALKYRRNDYQQYLDILSLEEFQAFHRKSKEAKKIDFAMLKREVIEQILK
KFQGEIKHHHHHH

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A Campylobacter jejuni β1-4GalNAcT (CjCgtA) variant comprising a polypeptide having at least 80% identity to the amino acid sequence set forth in SEQ ID NO: 1.

2. The CjCgtA variant of claim 1, comprising:
a mutation at one or more positions corresponding to G58, A110, and R166 in SEQ ID NO: 1, a mutation at P301 in SEQ ID NO: 1,
a mutation at one or more positions corresponding to L97 and Q244 in SEQ ID NO: 1,
a mutation at one or more positions corresponding to L142 and K229 in SEQ ID NO: 1,
a mutation at N107 in SEQ ID NO: 1,
a mutation at V50 in SEQ ID NO: 1,
a mutation at one or more positions corresponding to 0169, S212, S213, and A282 in SEQ ID NO: 1,
a mutation at G200 in SEQ ID NO: 1,
a mutation at one or more positions corresponding to D118, K286, and A296 in SEQ ID NO: 1,
a mutation at S287 in SEQ ID NO: 1,
a mutation at S243 in SEQ ID NO: 1,
a mutation at S193 in SEQ ID NO: 1,
a mutation at N124 in SEQ ID NO: 1,
a mutation at L80 in SEQ ID NO: 1,
a mutation at K46 in SEQ ID NO: 1,
a mutation at K288 in SEQ ID NO: 1,
a mutation at K35 in SEQ ID NO: 1,
a mutation at one or more positions corresponding to E170, F214, and I215 in SEQ ID NO: 1, or
a mutation at one or more positions corresponding to K111, S131, V190, R209, R210, V246, E289, and E304 in SEQ ID NO: 1.

3-20. (canceled)

21. The CjCgtA variant of claim 1, comprising a mutation at one or more positions corresponding to K35, K46, V50, G58, L80, L97, N107, A110, K111, D118, N124, S131, L142, R166, Q169, E170, V190, S193, G200, R209, R210, S212, S213, F214, I215, R218, K229, L231, S243, Q244, V246, A282, K286, S287, K288, E289, A296, P301, and E304 in SEQ ID NO: 1.

22. A Campylobacter jejuni β1-4GalNAcT (CjCgtA) variant comprising a polypeptide having at least 80% identity to the amino acid sequence set forth in SEQ ID NO: 2.

23. The CjCgtA variant of claim 22, comprising:
a polypeptide having at least 80% identity to the amino acid sequence set forth in SEQ ID NO: 2 wherein the polypeptide has a mutation at one or more positions corresponding to G43, A95, and R151 in SEQ ID NO: 2,
a mutation at P286 in SEQ ID NO: 2,
a mutation at one or more positions corresponding to L82 and Q229 in SEQ ID NO: 2,
a mutation at one or more positions corresponding to L127 and K214 in SEQ ID NO: 2,
a mutation at N92 in SEQ ID NO: 2,
a mutation at V35 in SEQ ID NO: 2,
a mutation at one or more positions corresponding to Q154, S197, S198, and A267 in SEQ ID NO: 2,
a mutation at G185 in SEQ ID NO: 2,
a mutation at one or more positions corresponding to D103, K271, and A281 in SEQ ID NO: 2,
a mutation at S272 in SEQ ID NO: 2,
a mutation at S228 in SEQ ID NO: 2,
a mutation at S178 in SEQ ID NO: 2,
a mutation at N109 in SEQ ID NO: 2,
a mutation at L65 in SEQ ID NO: 2,
a mutation at K31 in SEQ ID NO: 2,
a mutation at K273 in SEQ ID NO: 2,
a mutation at K20 in SEQ ID NO: 2,
a mutation at one or more positions corresponding to E155, F199, and 1200 in SEQ ID NO: 2, or
a mutation at one or more positions corresponding to K96, S116, V175, R194, R195, V231, E274, and E289 in SEQ ID NO: 2.

24-41. (canceled)

42. The CjCgtA variant of claim 22, a mutation at one or more positions corresponding to K20, K31, V35, G43, L65, L82, N92, A95, K96, D103, N109, S116, L127, R151, Q154, E155, V175, S178, G185, R194, R195, S197, S198, F199, I200, R203, K214, L216, S228, Q229, V231, A267, K271, S272, K273, E274, A281, P286, and E289 in SEQ ID NO: 2.

43. A Campylobacter jejuni β1-4GalNAcT (CjCgtA) variant comprising a polypeptide having at least 80% identity to the amino acid sequence set forth in SEQ ID NO: 23.

44. The Campylobacter jejuni β1-4GalNAcT (CjCgtA) variant of claim 43 a wherein the polypeptide has the amino acid sequence set forth in SEQ ID NO: 23.

45. The CjCgtA variant of claim 1, wherein the N-terminus of the polypeptide is fused to a maltose binding protein.

46. A Campylobacter jejuni β1-3-galactosyltransferase (CjCgtB) variant comprising a polypeptide having at least 80% identity to the amino acid sequence set forth in SEQ ID NO: 3.

47. The CjCgtB variant of claim 46, comprising:
a mutation at one or more positions corresponding to K26, S53, and K166 in SEQ ID NO: 3,
a mutation at one or more positions corresponding to Q15, I21, K26, N44, N47, S53, K57, I68, S83, I104, D108, E109, V116, K131, N135, S140, K142, T157, K166, E170, A173, M195, Q200, M205, N206, C207, N240, R243, and K260I in SEQ ID NO: 3,
a mutation at one or more positions corresponding to S53 and K166 in SEQ ID NO: 3,
a mutation at one or more positions corresponding to K26, S53, and C207 in SEQ ID NO: 3,
a mutation at one or more positions corresponding to S53 and C207 in SEQ ID NO: 3,
a mutation at one or more positions corresponding to K26, S53, K166, and C207 in SEQ ID NO: 3,
a mutation at one or more positions corresponding to S53, K166, and C207 in SEQ ID NO: 3,
a mutation at one or more positions corresponding to K26, S53, K166, A173, and C207 in SEQ ID NO: 3,
a mutation at one or more positions corresponding to K26, S53, K142, K131, K166, A173, Q200, and C207 in SEQ ID NO: 3,
a mutation at one or more positions corresponding to K26, S53, K142, K166, E170, A173, Q200, and C207 in SEQ ID NO: 3,
a mutation at one or more positions corresponding to Q15, I21, S53, K57, K142, K166, E170, A173, Q200, M250, and C207 in SEQ ID NO: 3,
a mutation at one or more positions corresponding to Q15, K26, S53, K57, K142, K166, I68, N135, E170, A173, Q200, C207, and N240 in SEQ ID NO: 3,
a mutation at one or more positions corresponding to K26, N44, S53, K57, I68, N135, K142, K166, E170, A173, Q200, C207, and N240 in SEQ ID NO: 3,
a mutation at one or more positions corresponding to Q15, K26, N44, S53, K57, N135, K142, K166, I68, E170, A173, Q200, C207, N240, and K260 in SEQ ID NO: 3,
a mutation at one or more positions corresponding to Q15, I21, K26, N44, S53, K57, I68, N135, K142, K166, E170, A173, Q200, C207, N240, and K260 in SEQ ID NO: 3,
a mutation at one or more positions corresponding to Q15, I21, K26, N44, S53, K57, I68, I104, N135, K142, K166, E170, A173, Q200, C207, N240, and K260 in SEQ ID NO: 3,
a mutation at one or more positions corresponding to I21, K26, S53, K57, I68, N44, I104, N135, D108, K142, K166, E170, A173, Q200, C207, N240, and K260 in SEQ ID NO: 3,
a mutation at one or more positions corresponding to I21, N44, N47, S53, K57, I104, D108, N135, 5140, K142, K166, I68, E170, A173, Q200, C207, N240, and K260 in SEQ ID NO: 3,
a mutation at one or more positions corresponding to I21, N44, N47, S53, K57, I68, I104, D108, V116, N135, 5140, K142, K166, E170, A173, Q200, C207, N240, and K260 in SEQ ID NO: 3,
a mutation at one or more positions corresponding to I21, N44, N47, S53, K57, I68, I104, D108, V116, N135, 5140, K142, K166, E170, A173, Q200, M205, C207, N240, and K260 in SEQ ID NO: 3,
a mutation at one or more positions corresponding to Q15, I21, N44, N47, S53, K57, N135, I68, I104, D108, E109, V116, S140, K142, K166, E170, A173, Q200, M205, N206, C207, N240, R243, and K260 in SEQ ID NO: 3,
a mutation at one or more positions corresponding to Q15, I21, K26, N44, N47, S53, K57, I68, S83, I104, D108, E109, V116, K131, N135, 5140, K142, T157, K166, E170, A173, M195, Q200, M205, N206, C207, N240, R243, and K260 in SEQ ID NO: 3, or
a mutation at position K26 in SEQ ID NO: 3.

48-69. (canceled)

70. The CjCgtB variant of claim 46, wherein the N-terminus of the polypeptide is fused to a maltose binding protein.

71. A polynucleotide encoding a CjCgtA variant according to of claim 1.

72. A host cell comprising the polynucleotide according to claim 71.

73. A reaction mixture comprising a CjCgtA variant according to claim 1.

74. The reaction mixture of claim 73, further comprising a glycosylation donor comprising a sugar component.

75. The reaction mixture of claim 73, further comprising a glycosylation acceptor comprising a sphingosine moiety.

76. The reaction mixture of claim 73, further comprising a detergent.

77. The reaction mixture of claim 76, wherein the detergent comprises an anionic detergent or a non-ionic detergent.

78. A method for preparing a glycosylated molecule, comprising:
forming a reaction mixture comprising (i) a glycosylation donor comprising a sugar component; (ii) a glycosylation acceptor comprising a sphingosine moiety; and (iii) a glycosyltransferase, wherein the glycosyltransferase is a CjCgtA variant according to claim 1; and
maintaining the reaction mixture under conditions suitable for forming a glycosylated molecule.

79. The method of claim 78, wherein the reaction mixture comprises a detergent.

80. The method of claim 79, wherein the detergent is an anionic detergent.

81. The method of claim 80, wherein the anionic detergent comprises sodium cholate.

82. The method of claim 81, wherein the detergent is a non-ionic detergent.

83. A polynucleotide encoding a CjCgtB variant according to claim 46.

84. A reaction mixture comprising a CjCgtB variant according to claim 46.

85. A method for preparing a glycosylated molecule, comprising:
forming a reaction mixture comprising (i) a glycosylation donor comprising a sugar component; (ii) a glycosylation acceptor comprising a sphingosine moiety; and (iii) a glycosyltransferase, wherein the glycosyltransferase is a CjCgtB variant according to claim 46; and
maintaining the reaction mixture under conditions suitable for forming a glycosylated molecule.
